(19) United States
(12) Patent Application Publication
(10) Pub. No.: US 2025/0349440 A1
(43) Pub. Date: Nov. 13, 2025

(54) Biomarker Panels for Predicting Multiple Sclerosis Disease Progression

(71) Applicant: Octave Bioscience, Inc., Menlo Park, CA (US)

(72) Inventors: Anisha Keshavan, San Diego, CA (US); Ati Ghoreyshi, San Francisco, CA (US); Ferhan Qureshi, Fremont, CA (US); William A. Hagstrom, Santa Barbara, CA (US)

(21) Appl. No.: 19/190,530

(22) Filed: Apr. 25, 2025

Related U.S. Application Data
(63) Continuation of application No. PCT/US2023/077800, filed on Oct. 25, 2023.
(60) Provisional application No. 63/419,227, filed on Oct. 25, 2022.
(60) Provisional application No. 63/589,266, filed on Oct. 10, 2023.

(51) Int. Cl. G16H 50/50; G16B 25/10; G16H 10/60; G16H 50/70

(52) U.S. Cl. CPC G16H 50/50; G16B 25/10; G16H 10/60; G16H 50/70

(57) ABSTRACT

Disclosed herein are methods for analyzing quantitative expression values of biomarkers of a biomarker panel for determining multiple sclerosis disease activity (e.g., multiple sclerosis disease progression) in a human subject. Further disclosed herein are kits for measuring quantitative expression values of the markers as well as computer systems and software embodiments of models for determining multiple sclerosis disease activity (e.g., multiple sclerosis disease progression) in human subjects based on the quantitative expression values of the markers.

### CROSS REFERENCE TO RELATED APPLICATIONS

This application claims the benefit of and priority to U.S. Provisional Patent Application No. 63/419,227, filed Oct. 25, 2022, and U.S. Provisional Patent Application No. 63/589,266, filed Oct. 10, 2023, the entire disclosure of each of which is hereby incorporated by reference in its entirety for all purposes.

### SUMMARY

Generally, MRI scans and/or clinical assessments (e.g., EDSS) are typically performed to determine MS disease progression in a subject. However, MRI scans are expensive and slow to perform. Higher-frequency measurement of the state of a patient's MS would allow for more nimble clinical management. Disclosed herein are methods for predicting multiple sclerosis disease activity (e.g., multiple sclerosis disease progression) using multivariate biomarker panels that analyze quantitative expression levels of biomarkers in samples obtained from the subject. Samples, such as samples obtained through blood draws, are simpler, faster, and cheaper than MRIs. Thus, analyzing expression levels of biomarkers, in conjunction with MRI volumetrics or just the biomarkers alone, in samples obtained from the subject can enable earlier detection and monitoring of MS disease progression.

Additionally disclosed herein are non-transitory computer readable mediums for predicting multiple sclerosis disease activity (e.g., multiple sclerosis disease progression) using multivariate biomarker panels. Additionally disclosed herein are kits containing a set of reagents for determining expression levels of multivariate biomarkers that are informative for predicting multiple sclerosis disease activity (e.g., multiple sclerosis disease progression). Additionally disclosed herein are systems for predicting multiple sclerosis disease activity (e.g., multiple sclerosis disease progression) using multivariate biomarker panels.

The advantages of a multivariate biomarker panel for detecting multiple sclerosis disease activity (e.g., multiple sclerosis disease progression) include the following:
Improved sensitivity: A multi-biomarker test improves performance (area under the curve (AUC (also referred to herein as AUROC), accuracy), especially by eliminating false negatives as that individual biomarkers are unable to detect
Detecting silent progression: A multi-biomarker test would enable detection of subclinical progression that manifests through radiographic atrophy, but does not manifest in worsening symptoms.
Specificity: Individual biomarkers are often differentially expressed in other neurologic conditions. A multi-biomarker test would help differentiate multiple sclerosis specific disease progression.
Predictive Power: Multivariate models incorporating shifts in biomarker levels identify patients heading towards increasing or decreasing active lesions (w/stronger performance than individual biomarkers alone).

Disclosed herein is a method for predicting multiple sclerosis disease progression in a subject, the method comprising: obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFAP, NEFL, CXCL-13, APLP1, MOG, OPG, VCAN, CDCP1, TNFSF13b, CNTN2, CXCL9, SERPINA9, OPN, CD6, TNFRSF10a, CCL20, FLRT2, COL4A1, GH, IL-12, PRTG, CXCL10, IL15, EGF, CXCL11, CFH, TNFSF10, IL18, IL6, TNF, HAVCR1, FLT3, MAN1A2, ACY3, ARHGEF1, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, GFRA2, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, and VEGFA; and generating a prediction of multiple sclerosis disease progression by applying a predictive model to the expression levels of the plurality of biomarkers.

Also disclosed herein is a method for predicting multiple sclerosis disease progression in a subject, the method comprising: obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, IL15, NEFL, GFAP, and VEGFA, and optionally comprises at least one or more of: CXCL-13, APLP1, MOG, OPG, VCAN, CDCP1, TNFSF13b, CNTN2, CXCL9, SERPINA9, OPN, CD6, TNFRSF10a, CCL20, COL4A1, GH, IL-12, PRTG, CXCL10, EGF, CXCL11, CFH, TNFSF10, IL18, IL6, TNF, CXCL13, NEFL, CCL20/MIP 3-α, FLRT2; and generating a prediction of multiple sclerosis disease progression by applying a predictive model to the expression levels of the plurality of biomarkers

In various embodiments, the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL. In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and GFAP. In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and NEFL. In various embodiments, a performance of the predictive model is characterized by an AUROC of at least 0.63, at least 0.66, at least 0.67, at least 0.69, at least 0.70, at least 0.74, at least 0.75, at least 0.78, or at least 0.81.

In various embodiments, the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2. In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2. In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2. In various embodiments, a performance of the predictive model is characterized by an AUROC of at least 0.68, at least 0.69, at least 0.70, at least 0.71, at least 0.72, at least 0.73, at least 0.74, at least 0.76, at least 0.77, at least 0.81, at least 0.82, at least 0.83, or at least 0.86.

In various embodiments, the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL. In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, and GFAP. In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, and NEFL. In various embodiments, a performance of the predictive model is characterized by an AUROC of at least 0.50, at least 0.51, at least 0.64, at least 0.70, at least 0.71, at least 0.72, at least 0.73, at least 0.74, at least 0.75, at least 0.76, at least 0.77, at least 0.79, at least 0.80, at least 0.81, at least 0.82, at least 0.83, at least 0.86, or at least 0.87.

In various embodiments, the method further comprises administering a therapy to the subject based on the prediction of multiple sclerosis disease progression.

Disclosed herein is a non-transitory computer readable medium comprising instructions that, when executed by a processor, cause the processor to: obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFAP, NEFL, CXCL-13, APLP1, MOG, OPG, VCAN, CDCP1, TNFSF13b, CNTN2, CXCL9, SERPINA9, OPN, CD6, TNFRSF10a, CCL20, FLRT2, COL4A1, GH, IL-12, PRTG, CXCL10, IL15, EGF, CXCL11, CFH, TNFSF10, IL18, IL6, TNF, HAVCR1, FLT3, MAN1A2, ACY3, ARHGEF1, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, GFRA2, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, and VEGFA; and generate a prediction of multiple sclerosis disease progression by applying a predictive model to the expression levels of the plurality of biomarkers.

Also disclosed herein is a non-transitory computer readable medium comprising instructions that, when executed by a processor, cause the processor to: obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, IL15, NEFL, GFAP, and VEGFA, and optionally comprises at least one or more of: CXCL-13, APLP1, MOG, OPG, VCAN, CDCP1, TNFSF13b, CNTN2, CXCL9, SERPINA9, OPN, CD6, TNFRSF10a, CCL20, COL4A1, GH, IL-12, PRTG, CXCL10, EGF, CXCL11, CFH, TNFSF10, IL18, IL6, TNF, CXCL13, NEFL, CCL20/MIP 3-α, FLRT2; and generate a prediction of multiple sclerosis disease progression by applying a predictive model to the expression levels of the plurality of biomarkers.

In various embodiments, the prediction of multiple sclerosis disease progression is a prediction of relapse associated worsening (RAW).

In various embodiments, the prediction of multiple sclerosis disease progression is a prediction of progression independent of relapse activity (PIRA).

In various embodiments, the prediction of multiple sclerosis disease progression is a measure of expanded disability status scale (EDSS) score. In various embodiments, an expanded disability status scale (EDSS) score less than 6 indicates a mild/moderate MS disease progression and a EDSS score greater than or equal to 6 indicates a severe MS disease progression.

In various embodiments, the prediction of multiple sclerosis disease progression is a measure of a patient determined disease steps (PDDS) score. In various embodiments, the prediction of multiple sclerosis disease progression is a patient determined disease steps (PDDS) score differentiating between severe and mild/moderate MS disease progression. In various embodiments, a patient determined disease steps (PDDS) score less than or equal to 4 indicates a mild/moderate MS disease progression and a PDDS score greater than 4 indicates a severe MS disease progression.

In various embodiments, the prediction of multiple sclerosis disease progression is a PRO measurement information system (PROMIS) score.

In various embodiments, the prediction of multiple sclerosis disease progression is a Multiple Sclerosis Rating Scale, Revised (MSRS-R) score.

In various embodiments, the prediction of multiple sclerosis disease progression is a MRI-based volumetric measurement. In various embodiments, the MRI-based volumetric measurement is one of whole brain atrophy, brain parenchymal fraction (BPF), white matter atrophy, or gray matter atrophy.

In various embodiments, the multiple sclerosis is one of relapsing-remitting multiple sclerosis (RRMS), secondary progressive multiple sclerosis (SPMS), primary-progressive multiple sclerosis (PPMS), progressive relapsing multiple sclerosis (PRMS), or clinically isolated syndrome (CIS).

In various embodiments, the dataset is derived from a sample obtained from the subject. In various embodiments, the sample is a blood, serum, or plasma sample. In various embodiments, obtaining or having obtained the dataset comprises performing one or more assays. In various embodiments, performing one or more assays comprises performing an immunoassay to determine the expression levels of the plurality of biomarkers. In various embodiments, the immunoassay is a Proximity Extension Assay (PEA) or LUMINEX xMAP Multiplex Assay.

### BRIEF DESCRIPTION OF THE DRAWINGS

These and other features, aspects, and advantages of the present invention will become better understood with regard to the following description and accompanying drawings.

FIG. 1A depicts an overview of an environment for assessing disease progression in a subject via a disease progression prediction system, in accordance with an embodiment.

FIG. 1B is an example block diagram of the disease progression system, in accordance with an embodiment.

FIG. 1C depicts an example set of training data, in accordance with an embodiment.

FIG. 2 illustrates an example computer for implementing the entities shown in FIGS. 1A-IC.

FIG. 3 shows patients' EDSS scores.

FIG. 4A shows serum protein detection between wPMS and stMS.

FIG. 4B shows serum protein detection between wPMS and stMS.

FIG. 5A shows serum protein detection between wPMS and stMS.

FIG. 5B shows serum protein detection between wPMS and stMS.

FIG. 6A shows serum protein detection as a function of grey matter volume in MS subjects.

FIG. 6B shows serum protein detection as a function of white matter volume in MS subjects.

FIG. 6C shows serum protein detection as a function of grey matter volume in MS subjects.

FIG. 6D shows serum protein detection as a function of white matter volume in MS subjects.

### DETAILED DESCRIPTION

### I. Definitions

Terms used in the claims and specification are defined as set forth below unless otherwise specified.

The term “subject” encompasses a cell, tissue, or organism, human or non-human, whether in vivo, ex vivo, or in vitro, male or female.

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “disease activity” encompasses the disease activity of any neurodegenerative disease including multiple sclerosis, Parkinson's Disease, Lewy body disease, Alzheimer's Disease, Amyotrophic lateral sclerosis (ALS), motor neuron disease, Huntington's Disease, Spinal muscular atrophy, Friedreich's ataxia, Batten disease,

The term “multiple sclerosis” or “MS” encompasses all forms of multiple sclerosis including relapsing-remitting multiple sclerosis (RRMS), secondary progressive multiple sclerosis (SPMS), primary-progressive multiple sclerosis (PPMS), progressive relapsing multiple sclerosis (PRMS), and clinically isolated syndrome (CIS).

The term “multiple sclerosis disease activity” or “disease activity of multiple sclerosis” as used herein refers to any of a diagnosis of multiple sclerosis (MS), a presence or absence of MS (e.g., general disease, subtle disease), a shift (e.g., increase or decrease) in the disease activity, disease progression, a severity of MS, a relapse or flare event associated with MS, a future or impending relapse or flare event, a rate of relapse (e.g., an annualized rate of relapse), a MS state (e.g., exacerbation or quiescence), a confirmation of no evidence of disease status, a response of a subject diagnosed with multiple sclerosis to a therapy, a degree of multiple sclerosis disability, a risk (e.g., likelihood) of the subject developing multiple sclerosis at a subsequent time, a change in multiple sclerosis disease in comparison to a prior measurement (e.g., longitudinal change in a patient relative to a baseline measurement), a measurable that is informative of the disease activity, or a differential diagnosis of a type of multiple sclerosis, including relapsing-remitting multiple sclerosis (RRMS), secondary progressive multiple sclerosis (SPMS), primary-progressive multiple sclerosis (PPMS), progressive relapsing multiple sclerosis (PRMS), and clinically isolated syndrome (CIS).

In one embodiment, the term “multiple sclerosis disease activity” or “disease activity of multiple sclerosis” refers to a diagnosis of multiple sclerosis (MS). In one embodiment, the term “multiple sclerosis disease activity” or “disease activity of multiple sclerosis” refers to a presence or absence of MS (e.g., general disease, subtle disease). In one embodiment, the term “multiple sclerosis disease activity” or “disease activity of multiple sclerosis” refers to a shift (e.g., increase or decrease) in the disease activity. In one embodiment, the term “multiple sclerosis disease activity” or “disease activity of multiple sclerosis” refers to a severity of MS. In one embodiment, the term “multiple sclerosis disease activity” or “disease activity of multiple sclerosis” refers to a relapse or flare event associated with MS. In one embodiment, the term “multiple sclerosis disease activity” or “disease activity of multiple sclerosis” refers to a future or impending relapse or flare event. In one embodiment, the term “multiple sclerosis disease activity” or “disease activity of multiple sclerosis” refers to a rate of relapse (e.g., an annualized rate of relapse). In one embodiment, the term “multiple sclerosis disease activity” or “disease activity of multiple sclerosis” refers to a MS state (e.g., exacerbation or quiescence). In one embodiment, the term “multiple sclerosis disease activity” or “disease activity of multiple sclerosis” refers to a confirmation of no evidence of disease status. In one embodiment, the term “multiple sclerosis disease activity” or “disease activity of multiple sclerosis” refers to a response of a subject diagnosed with multiple sclerosis to a therapy. In one embodiment, the term “multiple sclerosis disease activity” or “disease activity of multiple sclerosis” refers to a degree of multiple sclerosis disability. In one embodiment, the term “multiple sclerosis disease activity” or “disease activity of multiple sclerosis” refers to a risk (e.g., likelihood) of the subject developing multiple sclerosis at a subsequent time. In one embodiment, the term “multiple sclerosis disease activity” or “disease activity of multiple sclerosis” refers to a change in multiple sclerosis disease in comparison to a prior measurement (e.g., longitudinal change in a patient relative to a baseline measurement). In one embodiment, the term “multiple sclerosis disease activity” or “disease activity of multiple sclerosis” refers to a measurable that is informative of the disease activity. In some embodiments, the term “multiple sclerosis disease activity” or “disease activity of multiple sclerosis” is not inclusive of the progression of MS (e.g., MS disease progression). Specifically, in such embodiments as disclosed herein, biomarker panels used for predicting “multiple sclerosis disease activity” are distinct from biomarker panels used for predicting “multiple sclerosis disease progression.”

In various embodiments, measurables that are informative of the MS disease activity include measures of subtle disease activity (e.g., presence or absence of a specific number of gadolinium enhancing lesions e.g., exactly one lesion), general disease activity (e.g., presence or absence of 1 or more gadolinium enhancing lesion), a shift in disease activity (e.g., an appearance or disappearance of active gadolinium enhancing lesions), a severity of disease activity (e.g., a number of gadolinium enhancing lesions, where more gadolinium enhancing lesions is indicative of increased disease severity). In one embodiment, a measure that is informative of MS disease activity includes a measure of subtle disease activity (e.g., presence or absence of a specific number of gadolinium enhancing lesions e.g., exactly one lesion). In one embodiment, a measure that is informative of MS disease activity includes a measure of general disease activity (e.g., presence or absence of 1 or more gadolinium enhancing lesion). In one embodiment, a measure that is informative of MS disease activity includes a measure of a shift in disease activity (e.g., an appearance or disappearance of active gadolinium enhancing lesions). In one embodiment, a measure that is informative of MS disease activity includes a measure of a severity of disease activity (e.g., a number of gadolinium enhancing lesions, where more gadolinium enhancing lesions is indicative of increased disease severity).

In particular embodiments, the term “multiple sclerosis disease activity” or “disease activity of multiple sclerosis” refers to progression of MS (e.g., MS disease progression). In one embodiment, a measure that is informative of MS disease activity includes a measure of disease progression. Examples of measures of disease progression include the expanded disability status scale (EDSS), brain parenchymal fraction (BPF), atrophy measured by brain volume loss, or volumetrics by particular anatomical brain region. Additional measures of disease progression can include patient-reported outcome measures, such as patient determined disease steps (PDDS), PRO measurement information system (PROMIS), Multiple Sclerosis Rating Scale, Revised (MSRS-R), timed 25-foot walk (T25-FW), hand/arm function as measured by the 9-hole peg test (9-HPT), relapse associated worsening (RAW), or progression independent of relapse activity (PIRA).

In various embodiments, MS disease progression refers to advancing to milestones of MS disability, such as mild MS, moderate MS, or severe MS. Therefore, measures of MS disease progression can correspond to advancing to one or more of mild MS, moderate MS, or severe MS. For example, for a measure of MS disease progression that uses EDSS, an EDSS score less than 6 indicates mild/moderate MS disability and an EDSS score greater than or equal to 6 indicates severe MS disability. As another example, for a measure of MS disability that uses PDDS, a PDDS score less than equal to 4 indicates mild/moderate MS disability and a PDDS score greater than 4 indicates severe MS disability.

The terms “marker,” “markers,” “biomarker,” and “biomarkers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.).

The term “antibody” is used in the broadest sense and specifically covers monoclonal antibodies (including full length monoclonal antibodies), polyclonal antibodies, multispecific antibodies (e.g., bispecific antibodies), and antibody fragments that are antigen-binding so long as they exhibit the desired biological activity, e.g., an antibody or an antigen-binding fragment thereof.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

The term “biomarker panel” refers to a set biomarkers that are informative for predicting multiple sclerosis disease activity, and in particular embodiments, informative for predicting multiple sclerosis disease progression. For example, expression levels of the set of biomarkers in the biomarker panel can be informative for predicting multiple sclerosis disease progression. In various embodiments, a biomarker panel can include two, three, four, five, six, seven, eight, nine, ten eleven, twelve, thirteen, fourteen, fifteen, sixteen, seventeen, eighteen, nineteen, twenty, twenty one, twenty two, twenty three, twenty four, or twenty five biomarkers.

The term “obtaining a dataset associated with a sample” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

It must be noted that, as used in the specification, the singular forms “a,” “an” and “the” include plural referents unless the context clearly dictates otherwise.

### II. System Environment Overview

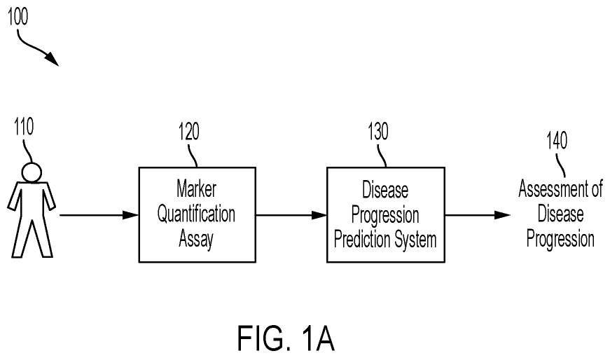

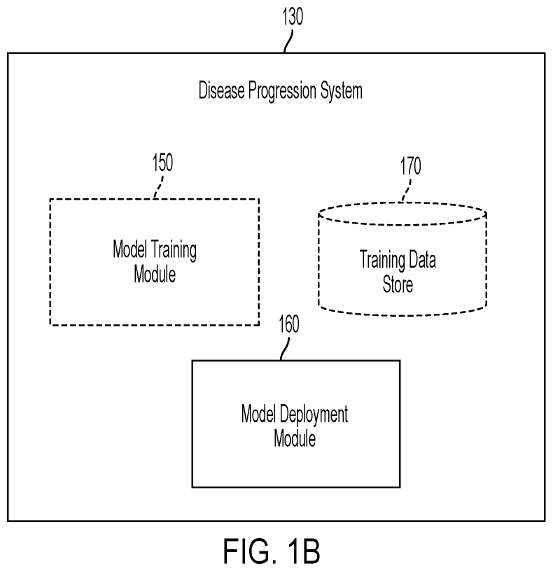

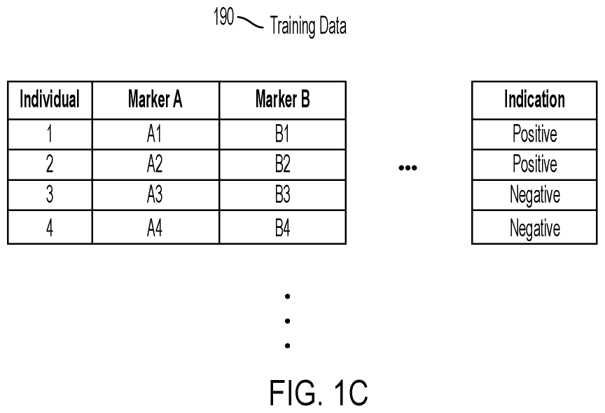

FIG. 1A depicts an overview of a system environment 100 for assessing disease progression in a subject, in accordance with an embodiment. The system environment 100 provides context in order to introduce a marker quantification assay 120 and an disease progression system 130.

In various embodiments, a test sample is obtained from the subject 110. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art.

The test sample is tested to determine values of one or more markers by performing the marker quantification assay 120. The marker quantification assay 120 determines quantitative expression values of one or more biomarkers from the test sample. The marker quantification assay 120 may be an immunoassay, and more specifically, a multi-plex immunoassay, examples of which are described in further detail below. The expression levels of various biomarkers can be obtained in a single run using a single test sample obtained from the subject 110. The quantified expression values of the biomarkers are provided to the disease progression system 130.

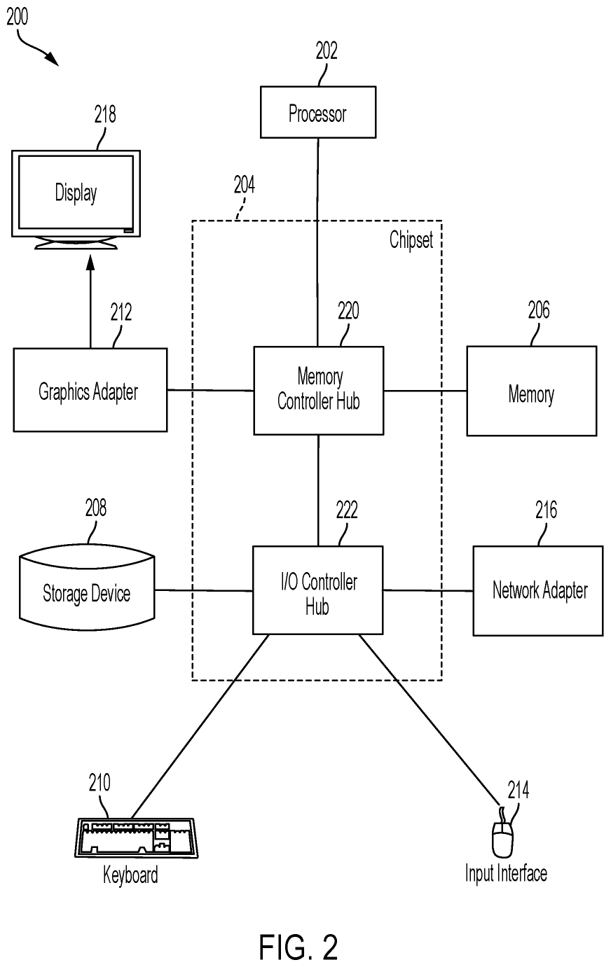

Generally, the disease progression system 130 includes one or more computers, embodied as a computer system 700 as discussed below with respect to FIG. 2. Therefore, in various embodiments, the steps described in reference to the disease progression system 130 are performed in silico. The disease progression system 130 analyzes the received biomarker expression values from the marker quantification assay 120 to generate an assessment of disease progression 140 in the subject 110.

In various embodiments, the marker quantification assay 120 and the disease progression system 130 can be employed by different parties. For example, a first party performs the marker quantification assay 120 which then provides the results to a second party which implements the disease progression system 130. For example, the first party may be a clinical laboratory that obtains test samples from subjects 110 and performs the assay 120 on the test samples. The second party receives the expression values of biomarkers resulting from the performed assay 120 analyzes the expression values using the disease progression system 130.

Reference is now made to FIG. 1B which depicts a block diagram illustrating the computer logic components of the disease progression system 130, in accordance with an embodiment. Specifically, the disease progression system 130 may include a model training module 150, a model deployment module 160, and a training data store 170.

Each of the components of the disease progression system 130 is hereafter described in reference to two phases: 1) a training phase and 2) a deployment phase. More specifically, the training phase refers to the building and training of one or more predictive models based on training data that includes quantitative expression values of biomarkers obtained from individuals that are known to be healthy, in a state of quiescence, in a state of remission, or in an earlier state of disease progression (e.g., mild/moderate MS as opposed to severe MS) or individuals that are known to have disease activity, in a state of exacerbation, in a state of relapse, or in a more advanced state of disease progression (e.g., severe MS as opposed to mild/moderate MS). Therefore, the predictive models are trained to predict disease activity in a subject based on quantitative biomarker expression values. During the deployment phase, a predictive model is applied to quantitative biomarker expression values from a test sample obtained from a subject of interest in order to generate a prediction of disease activity in the subject of interest.

In some embodiments, the components of the disease progression system 130 are applied during one of the training phase and the deployment phase. For example, the model training module 150 and training data store 170 (indicated by the dotted lines in FIG. 1B) are applied during the training phase whereas the model deployment module 160 is applied during the deployment phase. In various embodiments, the training phase and the deployment phase can be performed to enable continuously trained models. For example, the model training module 150 can train a model that the model deployment module 160 can subsequently deploy. The same model can undergo additional training by the model training module 150 (e.g., continuously trained using, for example, new training data that is obtained). Therefore, as the model is continuously trained, it can exhibit improved prediction capacity when analyzing samples during deployment.

In various embodiments, the components of the disease progression system 130 can be performed by different parties depending on whether the components are applied during the training phase or the deployment phase. In such scenarios, the training and deployment of the predictive model are performed by different parties. For example, the model training module 150 and training data store 170 applied during the training phase can be employed by a first party (e.g., to train a predictive model) and the model deployment module 160 applied during the deployment phase can be performed by a second party (e.g., to deploy the predictive model).

### III. Predictive Model

### III.A. Training a Predictive Model

During the training phase, the model training module 150 trains one or more predictive models using training data comprising expression values of biomarkers. Referring to FIG. 1B, the training data may be stored in the training data store 170. In various embodiments, the disease progression system 130 generates the training data comprising expression values of biomarkers by analyzing biomarker expression values in test samples. In various embodiments, the disease progression system 130 obtains the training data comprising expression values of biomarkers from a third party. The third party may have analyzed test samples to determine the biomarker expression values.

In various embodiments, the training data comprising expression values of biomarkers are derived from clinical subjects. For example, the training data can be expression values of biomarkers that were measured from test samples obtained from clinical subjects. Examples of expression values of biomarkers derived from clinical subjects include biomarker expression values obtained through clinical studies such as the multiple sclerosis CLIMB study (e.g., Comprehensive Longitudinal Investigation of Multiple Sclerosis at Brigham and Women's Hospital), the Accelerated Cure Project (ACP) for Multiple Sclerosis, and the Expression, Proteomics, Imaging, Clinical (EPIC) study at UCSF, the University Hospital Basel Cohort (UHBC), and the Prospective Investigation of Multiple Sclerosis in the Three Rivers Region (PROMOTE) study at the University of Pittsburgh.

In various embodiments, the training data further includes reference ground truths that indicate a disease activity, such as a multiple sclerosis disease activity. As an example, the training data includes reference ground truths that identify a presence or absence of multiple sclerosis (MS), a relapse or flare event associated with MS, a rate of relapse (e.g., an annualized rate of relapse), a MS state (e.g., exacerbation or quiescence), a response of a subject diagnosed with multiple sclerosis to a therapy, a degree of multiple sclerosis disability (e.g., a measure of multiple sclerosis disease progression such as mild, moderate, or severe MS), a risk (e.g., likelihood) of the subject developing multiple sclerosis at a subsequent time, or a measure of subtle disease activity (e.g., presence or absence of a specific number of gadolinium enhancing lesions e.g., one, two, three, or four lesions), or a measure of general disease activity (e.g., presence or absence of 1 or more gadolinium enhancing lesion). In particular embodiments, training data includes reference ground truths that identify a degree of multiple sclerosis disability (e.g., a measure of multiple sclerosis disease progression such as mild, moderate, or severe MS). In various embodiments, reference ground truths are generated by analyzing images (e.g., brain MRI images such as T1 or FLAIR images) captured from clinical subjects. Such images can be analyzed through computational means (e.g., image analysis algorithm) or can be manually analyzed. For example, images can be analyzed to determine a brain parenchymal fraction value, which is a known marker for MS disease progression. In various embodiments, the brain parenchymal fraction value of an image can serve as the reference ground truth. In various embodiments, the image analysis is performed by a third party and the reference ground truths can then be used for training the models described herein.

Reference is made to FIG. 1C, which depicts an example set of training data 190, in accordance with an embodiment. As shown in FIG. 1C, the training data 190 includes data corresponding to multiple individuals (e.g., column 1 depicting individual 1, 2, 3, 4 . . . ). For each individual, the training data 190 includes quantitative expression values (e.g., A1, B1, A2, B2, etc.) for different biomarkers obtained from the corresponding individual. In some embodiments, the quantitative expression values are determined by the marker quantification assay 120 shown in FIG. 1. Although FIG. 1C depicts 4 individuals and 2 different markers (marker A and marker B), the training data 190 may include tens, hundreds, or thousands of individuals as well as tens, hundreds, or thousands of markers.

As shown in FIG. 1C, a first training example (e.g., first row) of the training data refers to individual 1 and corresponding quantitative expression values of marker A (e.g., A1) and the quantitative expression value of marker B (e.g., B1). Similarly, the second training example (e.g., second row) of the training data refers to individual 2 and corresponding quantitative expression values of marker A (e.g., A2) and the quantitative expression value of marker B (e.g., B2). Individuals 3 and 4 have corresponding marker values as shown in FIG. 1C.

As shown in FIG. 1C, the training data 190 further includes a reference ground truth (“Indication” column) that identifies whether the corresponding individual has a positive or negative indication as to the disease activity. As an example, each indication may be an indication of multiple sclerosis disease progression in the patient. For example, referring to the first training example (e.g., first row), a “Positive” indication can reflect a presence of severe disease progression in individual 1. For example, a MRI scan of individual 1 may have revealed a presence of multiple gadolinium enhancing lesions. Similarly, an indication of a negative result (e.g., individual 3 or individual 4) reflects a presence of mild or moderate disease progression in the corresponding individual.

As another example, instead of the reference ground truth indicating a binary option (e.g., positive/negative), the reference ground truth may indicate one of multiple classes. For example, the reference ground truth may include a continuous range of values, wherein each value is indicative of one of the multiple classes. Specifically, the reference ground truth may include a value (e.g., value of “1”) which indicates that the corresponding individual has a presence of mild MS. A reference ground truth may include a value (e.g., value of “2”) which indicates that the corresponding individual has a presence of moderate MS. A reference ground truth may include a value (e.g., value of “3”) which indicates that the corresponding individual has a presence of severe MS. Additional values can be assigned that can further sub-divide the measures of disease progression into further classes.

In various embodiments, the reference ground truth may be a score, such as any of an EDSS score, a PDDS score, a PROMIS score, or a MSRS-R score. Therefore the reference ground truth score may itself be indicative of MS disease progression (e.g., PDDS score less or equal than 4 indicates mild/moderate MS whereas PDDS score greater than 4 indicates severe MS). Therefore, by training the predictive model using these reference ground truth scores, the predictive model is trained to predict a score (e.g., EDSS score, a PDDS score, a PROMIS score, or a MSRS-R score) for an individual that is indicative of MS disease progression.

In various embodiments, the reference ground truth may correspond to brain parenchymal fraction values that are derived from images captured from an individual, such as MRI images (T1 or FLAIR images). Here, brain parenchymal fraction is a known correlate to MS patients' disease progression. Thus, by training the predictive model using these reference ground truth scores, the predictive model is trained to predict a value that corresponds to brain parenchymal fraction values.

In various embodiments, the reference ground truth may indicate a particular class according to brain parenchymal fraction values derived from MRI images. MRI images can be analyzed and separated into different subsets according to brain parenchymal fraction values of the MRI images. For example, MRI images can be separated into 4 subsets (e.g., brain parenchymal fraction quartiles), where the first subset includes MRI images with the lowest range of brain parenchymal fraction values, the second subset includes MRI images with the next lowest range of brain parenchymal fraction values, the third subset includes MRI images with the third lowest range of brain parenchymal fraction values, and the fourth subset includes MRI images with the highest range of brain parenchymal fraction values. Thus, by training the predictive model using these reference ground truth scores, the predictive model can be trained to predict different classes according to predicted brain parenchymal fraction values.

In some embodiments, the model training module 150 retrieves the training data from the training data store 170 and randomly partitions the training data into a training set and a test set. As an example, 80% of the training data may be partitioned into the training set and the other 20% can be partitioned into the test set. Other proportions of training set and test set may be implemented. As such, the training set is used to train predictive models whereas the test set is used to validate the predictive models.

In various embodiments, the predictive model is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), linear mixed effects (LME) model, or any combination thereof. For example, the predictive model can be a stacked classifier that includes both a linear regression and decision tree.

The predictive model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the cellular disease model is trained using supervised learning algorithms, unsupervised learning algorithms, semi-supervised learning algorithms (e.g., partial supervision), weak supervision, transfer, multi-task learning, or any combination thereof.

In various embodiments, the predictive model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the cellular disease model are trained (e.g., adjusted) using the training data to improve the predictive capacity of the cellular disease model.

The model training module 150 trains one or more predictive models, each predictive model receiving, as input, one or more biomarkers. In various embodiments, the model training module 150 constructs a predictive model that receives, as input, expression values of two biomarkers. In various embodiments, the model training module 150 constructs a predictive model that receives, as input, expression values of three biomarkers. In various embodiments, the model training module 150 constructs a predictive model that receives, as input, expression values of four biomarkers. In some embodiments, the model training module 150 constructs a predictive model for more than four biomarkers. For example, a predictive model receives, as input, expression values of 2 biomarkers (e.g., 2 biomarkers categorized as Tier 1 in Table 6, 2 biomarkers categorized as Tier 2 in Table 7, or 2 biomarkers categorized as Tier 3 in Table 8). As another example, a predictive model receives, as input, expression values of 8 biomarkers (e.g., 8 biomarkers categorized as Tier 1 in Table 6, 8 biomarkers categorized as Tier 2 in Table 7, or 8 biomarkers categorized as Tier 3 in Table 8). As another example, the predictive model receives, as input, expression values of 17 biomarkers (e.g., 17 biomarkers categorized as Tier 1 in Table 6, 17 biomarkers categorized as Tier 2 in Table 7, or 17 biomarkers categorized as Tier 3 in Table 8).

In various embodiments, the model training module 150 identifies a set of biomarkers that are to be used to train a predictive model. The model training module 150 may begin with a list of candidate biomarkers that are promising for predicting disease activity (e.g., MS disease progression). In one embodiment, candidate biomarkers may be biomarkers identified through a literature curation process. In some embodiments, candidate biomarkers may be biomarkers whose expression values in test samples obtained from individuals that are positive for a disease activity (e.g., presence of MS, in an exacerbated state, in a state of severe MS, and the like) are statistically significant in comparison to expression values of biomarkers in test samples obtained from individuals that are negative for the disease activity.

In one embodiment, the model training module 150 performs a feature selection process to identify the set of biomarkers to be included in the biomarker panel. For example, the model training module 150 performs a sequential forward feature selection based on the expression values of the biomarkers and their importance in predicting a particular endpoint. For example, candidate biomarkers that are determined to be highly correlated with a particular disease activity endpoint (e.g., disease progression endpoint) would be deemed highly important are therefore likely to be included in the biomarker panel in comparison to other biomarkers that are not highly correlated with the disease activity endpoint (e.g., disease progression endpoint).

In some embodiments, the importance of each biomarker for a disease activity endpoint (e.g., disease progression endpoint) is determined by using a method including one of random forest (RF), gradient boosting (GBM), extreme gradient boosting (XGB), or LASSO algorithms. For example, if using random forest algorithms, the model training module 150 may generate a variable importance plot that depicts the importance of each candidate biomarker. Specifically, the random forest algorithm may provide, for each candidate biomarker, 1) a mean decrease in model accuracy and 2) a mean decrease in a Gini coefficient which is a measure of how much each candidate biomarker contributes to the homogeneity of nodes and leaves in the random forest. In one scenario, the importance of each candidate biomarker is dependent on one or both of the mean decrease in model accuracy and mean decrease in Gini coefficient. Each of GBM, XGB, and LASSO, can also be used to rank the importance of each candidate biomarker based on an influence value. Therefore, the model training module 150 can generate a ranking of each of candidate biomarkers using one of the methods including RF, GBM, XGB, or LASSO.

Each predictive model is iteratively trained using, as input, the quantitative expression values of the markers for each individual. For example, referring again to FIG. 1C, one iteration involves providing a training example (e.g., a row of the training data) that includes the quantitative expression value of biomarkers (e.g., “A1” and “B1”) for a particular individual (e.g., individual 1). Each predictive model is trained on reference ground truth data that includes the indication (e.g., the positive or negative result). In various embodiments, over training iterations, each predictive model is trained (e.g., the parameters are tuned) to minimize a prediction error between a prediction of MS activity (e.g., prediction of MS disease progression) outputted by the predictive model and the ground truth data. In various embodiments, the prediction error is calculated based on a loss function, examples of which include a L1 regularization (Lasso Regression) loss function, a L2 regularization (Ridge Regression) loss function, or a combination of L1 and L2 regularization (ElasticNet).

### III.B. Deploying a Predictive Model

During the deployment phase, the model deployment module 160 (as shown in FIG. 1B) analyzes quantitative biomarker expression values from a test sample obtained from a subject of interest by applying a trained predictive model. In some embodiments, the subject has not previously been diagnosed with a disease and therefore, the deployment of the predictive model enables in silico diagnosis of the disease based on the quantitative biomarker expression values derived from the subject. In some embodiments, the subject has been previously diagnosed with a disease. Here, the deployment of the predictive model enables in silico prediction of disease activity (e.g., disease progression) based on the quantitative biomarker expression values derived from the subject.

In various embodiments, the quantitative biomarker expression values are provided as input to the predictive model. The predictive model analyzes the quantitative biomarker expression values and outputs an assessment of disease activity (e.g., disease progression). The predicted score can then be informative of the disease activity. For example, the predicted score can enable the classification of the subject into one of multiple disease progression categories (e.g., one of mild/moderate disease progression or severe progression). In various embodiments, the assessment of disease activity (e.g., disease progression) is a predicted score representing the learned combination of the quantitative biomarker expression values. Generally, the predicted score represents an aggregation of the quantitative expression values and therefore, is not directly dependent on solely one biomarker expression value.

In various embodiments, the assessment of disease activity is a predicted score that may be informative of the disease activity in the subject. In various embodiments, the predicted score outputted by the prediction model is compared to one or more reference scores to determine a measure of the disease activity. Reference scores refer to previously determined scores, further described below as “healthy scores” or “diseased scores,” that correspond to diseased patients or non-diseased patients. For example, the one or more scores may be “healthy scores” corresponding to healthy patients, a patient's own baseline at a prior timepoint when the patient did not exhibit disease activity (e.g., longitudinal analysis), patients clinically diagnosed with the disease but not exhibiting disease activity, or a threshold score (e.g., a cutoff). As another example, the one or more scores may be “diseased scores” corresponding to diseased patients, a patient's own score indicating disease activity at a prior timepoint, or a threshold score (e.g., a cutoff). As one example, the threshold score can correspond to healthy patients and can be generated by training a predictive model using expression values of biomarkers from healthy patients. As another example, the threshold score can correspond to diseased patients and can be generated by training a predictive model using expression values of biomarkers from the diseased patients.

In various embodiments, a threshold score corresponding to healthy patients can be lower than a threshold score corresponding to diseased patients. For example, the threshold score corresponding to healthy patients can be at least 5% lower than a threshold score corresponding to diseased patients. As another example, the threshold score corresponding to healthy patients can be at least 10% lower than a threshold score corresponding to diseased patients. As another example, the threshold score corresponding to healthy patients can be at least 15% lower than a threshold score corresponding to diseased patients. As another example, the threshold score corresponding to healthy patients can be at least 20% lower than a threshold score corresponding to diseased patients. As another example, the threshold score corresponding to healthy patients can be at least 25% lower than a threshold score corresponding to diseased patients. As another example, the threshold score corresponding to healthy patients can be at least 50% lower than a threshold score corresponding to diseased patients. As another example, the threshold score corresponding to healthy patients can be at least 75% lower than a threshold score corresponding to diseased patients.

In various embodiments, a threshold score corresponding to healthy patients can be higher than a threshold score corresponding to diseased patients. For example, the threshold score corresponding to healthy patients can be at least 5% higher than a threshold score corresponding to diseased patients. As another example, the threshold score corresponding to healthy patients can be at least 10% higher than a threshold score corresponding to diseased patients. As another example, the threshold score corresponding to healthy patients can be at least 15% higher than a threshold score corresponding to diseased patients. As another example, the threshold score corresponding to healthy patients can be at least 20% higher than a threshold score corresponding to diseased patients. As another example, the threshold score corresponding to healthy patients can be at least 25% higher than a threshold score corresponding to diseased patients. As another example, the threshold score corresponding to healthy patients can be at least 50% higher than a threshold score corresponding to diseased patients. As another example, the threshold score corresponding to healthy patients can be at least 75% higher than a threshold score corresponding to diseased patients. As another example, the threshold score corresponding to healthy patients can be at least 100% higher than a threshold score corresponding to diseased patients. Thus, in particular embodiments, the predicted score outputted by the prediction model is compared to one or both of the threshold score corresponding to healthy patients and threshold score corresponding to diseased patients, and based on the comparison, a measure of the disease activity is determined.

In various embodiments, the assessment of disease activity corresponds to the presence of absence of disease. In one embodiment, the predicted score outputted by the prediction model can be compared to a healthy score. The subject can be classified as having the disease if the predicted score of the subject is significantly different (e.g., p-value <0.05) in comparison to the healthy score. In one embodiment, the predicted score outputted by the prediction model can be compared to the diseased score. The subject can be classified as not having the disease if the predicted score of the subject is significantly different (e.g., p-value <0.05) in comparison to the diseased score. In some embodiments, the predicted score outputted by the prediction model is compared to both the healthy score and the diseased score. For example, the subject can be classified as having the disease if the predicted score of the subject is significantly different (e.g., p-value <0.05) in comparison to the healthy scores and not significantly different (e.g., p-value >0.05 in comparison to the diseased scores for patients that have been diagnosed with the disease. In various embodiments, depending on the classification of the subject, the subject can undergo treatment. In other words, the assessment can guide the treatment of the subject. For example, if the subject is classified as having the disease, the subject can be administered a therapeutic intervention to treat the disease.

In various embodiments, the assessment of disease activity corresponds to the presence of absence of subtle disease. In one embodiment, the predicted score outputted by the prediction model can be compared to a score corresponding to individuals previously determined to have a presence of subtle disease (e.g., a specific number of gadolinium enhancing lesion on a MRI scan e.g., exactly one lesion). The subject can be classified as having subtle disease if the predicted score of the subject is not significantly different (e.g., p-value >0.05) in comparison to the score corresponding to individuals previously determined to have a presence of subtle disease. The subject can be classified as having subtle disease if the predicted score of the subject is significantly different (e.g., p-value <0.05) in comparison to the score corresponding to individuals previously determined to not have a presence of subtle disease. In one embodiment, the predicted score outputted by the prediction score is compared to a score corresponding to individuals without subtle disease (e.g., zero gadolinium enhancing lesions on a MRI scan). The subject can be classified as not having subtle disease if the predicted score of the subject is not significantly different (e.g., p-value >0.05) from the score corresponding to individuals that do not have subtle disease (e.g., zero gadolinium enhancing lesion on a MRI scan). Alternatively, the subject can be classified as having subtle disease if the predicted score of the subject is significantly different (e.g., p-value <0.05) from the score corresponding to individuals that do not have subtle disease (e.g., zero gadolinium enhancing lesion on a MRI scan).

In some embodiments, the predicted score outputted by the prediction model is compared to both a score corresponding to individuals previously determined to have a presence of subtle disease (e.g., a particular number of gadolinium enhancing lesions on a MRI scan) and a score corresponding to individuals without subtle disease (e.g., zero gadolinium enhancing lesions on a MRI scan). For example, the subject can be classified as having subtle disease if the predicted score of the subject is significantly different (e.g., p-value <0.05) in comparison to the score corresponding to individuals without subtle disease (e.g., zero gadolinium enhancing lesions on a MRI scan) and not significantly different (e.g., p-value >0.05) in comparison to the score corresponding to individuals previously determined to have a presence of subtle disease (e.g., a particular number of gadolinium enhancing lesions on a MRI scan e.g., exactly one gadolinium enhancing lesion).

In various embodiments, the assessment of disease activity corresponds to the presence of absence of general disease. In one embodiment, the predicted score outputted by the prediction model can be compared to a score corresponding to individuals previously determined to have a presence of general disease (e.g., one or more gadolinium enhancing lesions on a MRI scan). The subject can be classified as having general disease if the predicted score of the subject is significantly different (e.g., p-value <0.05) in comparison to the score corresponding to individuals previously determined to not have a presence of general disease. The subject can be classified as not having general disease if the predicted score of the subject is not significantly different (e.g., p-value >0.05) in comparison to the score corresponding to individuals previously determined to not have a presence of general disease. In one embodiment, the predicted score outputted by the prediction score is compared to a score corresponding to individuals without general disease (e.g., zero gadolinium enhancing lesions on a MRI scan). The subject can be classified as not having general disease if the predicted score of the subject is not significantly different (e.g., p-value >0.05) from the score corresponding to individuals that do not have general disease (e.g., zero gadolinium enhancing lesion on a MRI scan). The subject can be classified as having general disease if the predicted score of the subject is significantly different (e.g., p-value <0.05) from the score corresponding to individuals that do not have general disease (e.g., zero gadolinium enhancing lesion on a MRI scan). In some embodiments, the predicted score outputted by the prediction model is compared to both a score corresponding to individuals previously determined to have a presence of general disease (e.g., one or more gadolinium enhancing lesions on a MRI scan) and a score corresponding to individuals without general disease (e.g., zero gadolinium enhancing lesions on a MRI scan). For example, the subject can be classified as having general disease if the predicted score of the subject is significantly different (e.g., p-value <0.05) in comparison to the score corresponding to individuals without general disease (e.g., zero gadolinium enhancing lesions on a MRI scan) and not significantly different (e.g., p-value >0.05) in comparison to the score corresponding to individuals previously determined to have a presence of general disease (e.g., one or more gadolinium enhancing lesions on a MRI scan).

In various embodiments, the assessment of disease activity corresponds to the directional shift in disease activity based on a predicted increase or decrease in the number of gadolinium enhancing lesions. In one embodiment, the predicted score outputted by the prediction model can be compared to a score corresponding to individuals previously determined to have undergone an increase in disease activity (e.g., increasing numbers of gadolinium enhancing lesions on a MRI scan). The subject can be classified as likely to encounter an increase in disease activity if the predicted score of the subject is significantly different (e.g., p-value <0.05) in comparison to the score corresponding to individuals previously determined to not have undergone an increase in disease activity. The subject can be classified as unlikely to encounter an increase in disease activity if the predicted score of the subject is not significantly different (e.g., p-value >0.05) in comparison to the score corresponding to individuals previously determined to not have undergone an increase in disease activity. In one embodiment, the predicted score outputted by the prediction score is compared to a score corresponding to individuals previously determined to have undergone a decrease in disease activity (e.g., decreasing numbers of gadolinium enhancing lesions on a MRI scan). The subject can be classified as likely to encounter a decrease in disease activity if the predicted score of the subject is not significantly different (e.g., p-value >0.05) from the score corresponding to individuals that have encountered a decrease in disease activity. The subject can be classified as likely to encounter a decrease in disease activity if the predicted score of the subject is significantly different (e.g., p-value <0.05) from the score corresponding to individuals that have not encountered a decrease in disease activity. In some embodiments, the predicted score outputted by the prediction model is compared to both a score corresponding to individuals previously determined to have undergone an increase in disease activity (e.g., increasing numbers of gadolinium enhancing lesions on a MRI scan) and a score corresponding to individuals who have undergone a decrease in disease activity (e.g., decreasing numbers of gadolinium enhancing lesions on a MRI scan). For example, the subject can be classified as likely to undergo an increase in disease activity if the predicted score of the subject is significantly different (e.g., p-value <0.05) in comparison to the score corresponding to individuals who have undergone a decrease in disease activity (e.g., decreasing number of gadolinium enhancing lesions on a MRI scan) and not significantly different (e.g., p-value >0.05) in comparison to the score corresponding to individuals who have undergone an increase in disease activity (e.g., increasing numbers of gadolinium enhancing lesions on a MRI scan). In various embodiments, the subject can be classified as unlikely to encounter either an increase or decrease in disease activity (e.g., the disease activity in the subject is stable) if the predicted score of the subject is not significantly different (e.g., p-value >0.05) in comparison to both the score corresponding to individuals who have undergone an increase in disease activity and the score corresponding to individuals who have undergone a decrease in disease activity.

In various embodiments, the assessment of disease activity corresponds to a state of disease in a subject. For example, if the disease is MS, the state of disease in the subject is one of quiescent vs exacerbation. In one embodiment, the predicted score outputted by the prediction model can be compared to a score corresponding to individuals previously determined to be in a quiescent state (e.g., clinically determined to be in a quiescent state). The subject can be classified as being in a quiescent state if the predicted score of the subject is significantly different (e.g., p-value <0.05) in comparison to the score corresponding to individuals previously determined to not be in a quiescent state. The subject can be classified as not being in a quiescent state if the predicted score of the subject is not significantly different (e.g., p-value >0.05) in comparison to the score corresponding to individuals previously determined to be in a quiescent state. In one embodiment, the predicted score outputted by the prediction score is compared to a score corresponding to individuals previously determined to be in an exacerbated state. The subject can be classified as being in an exacerbated state if the predicted score of the subject is not significantly different (e.g., p-value >0.05) from the score corresponding to individuals previously determined to be in an exacerbated state. The subject can be classified as not being in an exacerbated state if the predicted score of the subject is significantly different (e.g., p-value <0.05) from the score corresponding to individuals previously determined to be in an exacerbated state. In some embodiments, the predicted score outputted by the prediction model is compared to both a score corresponding to individuals previously determined to be in a quiescent state and a score corresponding to individuals in an exacerbated state. For example, the subject can be classified as being in an exacerbated state if the predicted score of the subject is significantly different (e.g., p-value <0.05) in comparison to the score corresponding to individuals in a quiescent state and not significantly different (e.g., p-value >0.05) in comparison to the score corresponding to individuals previously determined to be in an exacerbated state.

In various embodiments, the assessment of disease activity corresponds to a likely response to a therapy of provided to the subject. In one embodiment, the predicted score outputted by the prediction model can be compared to a score corresponding to individuals previously determined to be responsive to the therapy (e.g., clinically determined to be responsive to the therapy). The subject can be classified as being a responder if the predicted score of the subject is significantly different (e.g., p-value <0.05) in comparison to the score corresponding to individuals previously determined to not be responsive to the therapy. The subject can be classified as being a responder if the predicted score of the subject is not significantly different (e.g., p-value >0.05) in comparison to the score corresponding to individuals previously determined to be responsive to the therapy. In one embodiment, the predicted score outputted by the prediction score is compared to a score corresponding to individuals previously determined to be non-responders. The subject can be classified as a non-responder if the predicted score of the subject is not significantly different (e.g., p-value >0.05) from the score corresponding to individuals previously determined to be non-responders. The subject can be classified as a non-responder if the predicted score of the subject is significantly different (e.g., p-value <0.05) from the score corresponding to individuals previously determined to be responders. In some embodiments, the predicted score outputted by the prediction model is compared to both a score corresponding to individuals previously determined to be responders and a score corresponding to individuals previously determined to be non-responders. For example, the subject can be classified as being a responder if the predicted score of the subject is significantly different (e.g., p-value <0.05) in comparison to the score corresponding to individuals previously determined to be non-responders and not significantly different (e.g., p-value >0.05) in comparison to the score corresponding to individuals previously determined to be responders.

In various embodiments, the assessment of disease activity is a classification of disease progression (e.g., mild/moderate disease versus severe disability). Thus, in such embodiments, the predicted score outputted by the prediction model can be compared to one or both scores corresponding to individuals previously identified as having mild/moderate disease and corresponding to individuals previously identified as having severe disability.

In various embodiments, a score corresponding to individuals previously identified as having mild/moderate disease can be lower than a score corresponding to individuals previously identified as having severe disability. For example, the score corresponding to individuals previously identified as having mild/moderate disease can be at least 5% lower than a score corresponding to individuals previously identified as having severe disability. As another example, the score corresponding to individuals previously identified as having mild/moderate disease can be at least 10% lower than a score corresponding to individuals previously identified as having severe disability. As another example, the score corresponding to individuals previously identified as having mild/moderate disease can be at least 15% lower than a score corresponding to individuals previously identified as having severe disability. As another example, the score corresponding to individuals previously identified as having mild/moderate disease can be at least 20% lower than a score corresponding to individuals previously identified as having severe disability. As another example, the score corresponding to individuals previously identified as having mild/moderate disease can be at least 25% lower than a score corresponding to individuals previously identified as having severe disability. As another example, the score corresponding to individuals previously identified as having mild/moderate disease can be at least 50% lower than a score corresponding to individuals previously identified as having severe disability. As another example, the score corresponding to individuals previously identified as having mild/moderate disease can be at least 75% lower than a score corresponding to individuals previously identified as having severe disability.

In various embodiments, the score corresponding to individuals previously identified as having mild/moderate disease can be higher than a score corresponding to individuals previously identified as having severe disability. For example, the score corresponding to individuals previously identified as having mild/moderate disease can be at least 5% higher than a score corresponding to individuals previously identified as having severe disability. As another example, the score corresponding to individuals previously identified as having mild/moderate disease can be at least 10% higher than a score corresponding to individuals previously identified as having severe disability. As another example, the score corresponding to individuals previously identified as having mild/moderate disease can be at least 15% higher than a score corresponding to individuals previously identified as having severe disability. As another example, the score corresponding to individuals previously identified as having mild/moderate disease can be at least 20% higher than a score corresponding to individuals previously identified as having severe disability. As another example, the score corresponding to individuals previously identified as having mild/moderate disease can be at least 25% higher than a score corresponding to individuals previously identified as having severe disability. As another example, the score corresponding to individuals previously identified as having mild/moderate disease can be at least 50% higher than a score corresponding to individuals previously identified as having severe disability. As another example, the score corresponding to individuals previously identified as having mild/moderate disease can be at least 75% higher than a score corresponding to individuals previously identified as having severe disability. As another example, the score corresponding to individuals previously identified as having mild/moderate disease can be at least 100% higher than a score corresponding to individuals previously identified as having severe disability. Thus, in particular embodiments, the predicted score outputted by the prediction model is compared to one or both of the score corresponding to individuals previously identified as having mild/moderate disease and score corresponding to individuals previously identified as having severe disability, and based on the comparison, a measure of the disease progression is determined.

In one embodiment, the assessment of disease activity is an assessment of disease progression and can correspond to a degree of MS disability in a subject diagnosed with multiple sclerosis. In one embodiment, the degree of MS disability corresponds to an EDSS score or to a range of EDSS scores. In various embodiments, the assessment (e.g., predicted score) corresponding to the subject is compared to multiple reference scores. Each reference score may correspond to a group of individuals that have been clinically categorized in a degree of disability. In various embodiments, a reference score is an EDSS score. In various embodiments, a reference score corresponds to an EDSS score. For example, a first reference score may correspond to individuals clinically categorized with a score of 1 on the EDSS. Additional reference scores may correspond to groups of individuals that have been clinically categorized with a score of 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0. As another example, a first reference score may correspond to individuals previously categorized with a score between 1 and 6 on the EDSS scale and a second reference score may correspond to individuals previously categorized with a score between 6.5 and 10 on the EDSS scale. In one scenario, the subject may be classified with one of the EDSS scores if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a PDDS score or a range of PDDS scores. In various embodiments, the assessment (e.g., predicted score) corresponding to the subject is compared to multiple reference scores. Each score may correspond to a group of individuals that have been clinically categorized in a degree of disability. In various embodiments, a reference score is a PDDS score. In various embodiments, a reference score corresponds to a PDDS score. For example, a first reference score may correspond to individuals previously categorized with a score of 1 on the PDDS scale. Additional reference scores may correspond to groups of individuals that have been previously categorized with a score of 2, 3, 4, 5, 6, 7, or 8. As another example, a first reference score may correspond to individuals previously categorized with a score between 1 and 4 on the PDDS scale and a second reference score may correspond to individuals previously categorized with a score between 5 and 8 on the PDDS scale. In one scenario, the subject may be classified with one of the PDDS scores if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a brain parenchymal fraction score. In various embodiments, the assessment (e.g., predicted score) corresponding to the subject is compared to one or more reference scores. For example, a first reference score may be a brain parenchymal fraction score corresponding to individuals previously categorized as having mild/moderate MS. A second reference score may be a brain parenchymal fraction score corresponding to individuals previously categorized as having severe MS. In one scenario, the subject may be classified with having mild/moderate or severe MS if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a PROMIS score. In various embodiments, the assessment (e.g., predicted score) corresponding to the subject is compared to one or more reference scores. For example, a first reference score may be a PROMIS score corresponding to individuals previously categorized as having mild/moderate MS. A second reference score may be a PROMIS score corresponding to individuals previously categorized as having severe MS. In one scenario, the subject may be classified with having mild/moderate or severe MS if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In some embodiments, the degree of MS disability corresponds to a MSRS-R score. In various embodiments, the assessment (e.g., predicted score) corresponding to the subject is compared to one or more reference scores. For example, a first reference score may be a MSRS-R score corresponding to individuals previously categorized as having mild/moderate MS. A second reference score may be a MSRS-R score corresponding to individuals previously categorized as having severe MS. In one scenario, the subject may be classified with having mild/moderate or severe MS if the subject's predicted score outputted by the prediction model is not significantly different (e.g., p-value >0.05) from one group and is significantly different (e.g., p-value <0.05) in comparison to all other groups. The subject may be treated according to clinical protocols based on the categorization.

In one embodiment, the assessment of disease activity corresponds to a risk (e.g., likelihood) of the subject developing a disease at a subsequent time. In various embodiments, the assessment (e.g., predicted score) corresponding to the subject is compared to multiple scores. Each score may correspond to a group of individuals in a risk group that have been clinically categorized with a particular risk of developing MS. As an example, the risk groups may be divided into a high risk group, medium risk group, and low risk group. In one scenario, the subject may be classified in a risk group if the subject's predicted score is not significantly different (e.g., p-value >0.05) from one group and is significantly different (e.g., p-value <0.05) in comparison to other groups. Therefore, the subject can undertake changes in lifestyle and/or treatments based on the prediction of a risk/likelihood of developing MS.

In various embodiments, a measure of the disease activity predicted by the predictive model provides additional utility for managing the disease activity in the patient. As one example, the measure of the disease activity predicted by the predictive model is useful for selecting a candidate therapeutic or for determining the effectiveness of a previously administered therapeutic.

In various embodiments, the measure of disease activity predicted by the predictive model for a patient can be compared to a prior measure of disease activity to determine whether a therapeutic administered to the patient is demonstrating efficacy. As one example, the prior measure of disease activity may be a prediction determined for the same patient (e.g., a baseline measure of disease activity). Thus in this example, the comparison of the measure of disease activity and the prior measure of disease activity is a longitudinal analysis of a patient that is undergoing treatment using the therapeutic. As such, a difference or lack of difference between the measure of disease activity and prior measure of disease activity can be an indication that the therapeutic is having an effect or lack of an effect. As another example, the prior measure of disease activity may be a measure determined for a population of patients (e.g., a reference set of patients). In this example, the comparison of the measure of disease activity and the prior measure of disease activity can reveal whether the patient is experiencing effects due to a therapeutic, as evidenced by the measure of disease activity, in comparison to the prior measure of disease activity for the population of patients.

In various embodiments, if the comparison between the measure of disease activity and prior measure of disease activity indicates that a currently administered therapeutic is not exhibiting an effect, or is not exhibiting an effect to a desired extent, a change in the patient's treatment can be undertaken. In one embodiment, the treatment dose of the currently administered therapeutic can be altered to effect a patient response. For example, the currently administered therapeutic can be increased in dosage. In one embodiment, a candidate therapeutic can be selected for administration to the patient. In various embodiments, a candidate therapeutic can be administered to the patient in place of the currently administered therapeutic or the candidate therapeutic can be administered to the patient in addition to the currently administered therapeutic.

As another example, a measure of the disease activity is useful for supporting symptom and medication tracking, nursing interventions, laboratory monitoring, and curated longitudinal MRI reports. In such scenarios, the measure of disease activity can reduce unplanned healthcare utilization (e.g., unplanned visits to physician's office), thereby improving patient and physician satisfaction.

In various embodiments, a performance of the predictive model is characterized by an AUROC of at least 0.50, at least 0.51, at least 0.52, at least 0.53, at least 0.54, at least 0.55, at least 0.56, at least 0.57, at least 0.58, at least 0.59, at least 0.60, at least 0.61, at least 0.62, at least 0.63, at least 0.64, at least 0.65, at least 0.66, at least 0.67, at least 0.68, at least 0.69, at least 0.70, at least 0.71, at least 0.72, at least 0.73, at least 0.74, at least 0.75, at least 0.76, at least 0.77, at least 0.78, at least 0.79, at least 0.80, at least 0.81, at least 0.82, at least 0.83, at least 0.84, at least 0.85, at least 0.86, at least 0.87, at least 0.88, at least 0.89, and at least 0.90. In various embodiments, a performance of the predictive model is characterized by an AUROC of at least 0.50. In various embodiments, a performance of the predictive model is characterized by an AUROC of at least 0.51. In various embodiments, a performance of the predictive model is characterized by an AUROC of at least 0.52. In various embodiments, a performance of the predictive model is characterized by an AUROC of at least 0.53. In various embodiments, a performance of the predictive model is characterized by an AUROC of at least 0.54. In various embodiments, a performance of the predictive model is characterized by an AUROC of at least 0.55. In various embodiments, a performance of the predictive model is characterized by an AUROC of at least 0.56. In various embodiments, a performance of the predictive model is characterized by an AUROC of at least 0.57. In various embodiments, a performance of the predictive model is characterized by an AUROC of at least 0.58. In various embodiments, a performance of the predictive model is characterized by an AUROC of at least 0.59. In various embodiments, a performance of the predictive model is characterized by an AUROC of at least 0.60. In various embodiments, a performance of the predictive model is characterized by an AUROC of at least 0.61. In various embodiments, a performance of the predictive model is characterized by an AUROC of at least 0.62. In various embodiments, a performance of the predictive model is characterized by an AUROC of at least 0.63. In various embodiments, a performance of the predictive model is characterized by an AUROC of at least 0.64. In various embodiments, a performance of the predictive model is characterized by an AUROC of at least 0.65. In various embodiments, a performance of the predictive model is characterized by an AUROC of at least 0.66. In various embodiments, a performance of the predictive model is characterized by an AUROC of at least 0.67. In various embodiments, a performance of the predictive model is characterized by an AUROC of at least 0.68. In various embodiments, a performance of the predictive model is characterized by an AUROC of at least 0.69. In various embodiments, a performance of the predictive model is characterized by an AUROC of at least 0.70. In various embodiments, a performance of the predictive model is characterized by an AUROC of at least 0.71. In various embodiments, a performance of the predictive model is characterized by an AUROC of at least 0.72. In various embodiments, a performance of the predictive model is characterized by an AUROC of at least 0.73. In various embodiments, a performance of the predictive model is characterized by an AUROC of at least 0.74. In various embodiments, a performance of the predictive model is characterized by an AUROC of at least 0.75. In various embodiments, a performance of the predictive model is characterized by an AUROC of at least 0.76. In various embodiments, a performance of the predictive model is characterized by an AUROC of at least 0.77. In various embodiments, a performance of the predictive model is characterized by an AUROC of at least 0.78. In various embodiments, a performance of the predictive model is characterized by an AUROC of at least 0.79. In various embodiments, a performance of the predictive model is characterized by an AUROC of at least 0.80. In various embodiments, a performance of the predictive model is characterized by an AUROC of at least 0.81. In various embodiments, a performance of the predictive model is characterized by an AUROC of at least 0.82. In various embodiments, a performance of the predictive model is characterized by an AUROC of at least 0.83. In various embodiments, a performance of the predictive model is characterized by an AUROC of at least 0.84. In various embodiments, a performance of the predictive model is characterized by an AUROC of at least 0.85. In various embodiments, a performance of the predictive model is characterized by an AUROC of at least 0.86. In various embodiments, a performance of the predictive model is characterized by an AUROC of at least 0.87. In various embodiments, a performance of the predictive model is characterized by an AUROC of at least 0.88. In various embodiments, a performance of the predictive model is characterized by an AUROC of at least 0.89. In various embodiments, a performance of the predictive model is characterized by an AUROC of at least 0.90.

### IV. Biomarker Panel

In various embodiments, the assessment of disease activity (e.g., disease progression) involves implementing a univariate biomarker panel. Therefore, the univariate biomarker panel includes one biomarker. In other embodiments, the assessment of disease activity (e.g., disease progression) involves implementing a multivariate biomarker panel. In such embodiments, the multivariate biomarker panel includes more than one biomarker. In various embodiments, the multivariate biomarker panel includes two biomarkers. In various embodiments, the multivariate biomarker panel includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 biomarkers. In particular embodiments, the multivariate biomarker panel includes 2 biomarkers. In particular embodiments, the multivariate biomarker panel includes 3 biomarkers. In particular embodiments, the multivariate biomarker panel includes 4 biomarkers. In particular embodiments, the multivariate biomarker panel includes 5 biomarkers. In particular embodiments, the multivariate biomarker panel includes 6 biomarkers. In particular embodiments, the multivariate biomarker panel includes 7 biomarkers. In particular embodiments, the multivariate biomarker panel includes 8 biomarkers. In particular embodiments, the multivariate biomarker panel includes 9 biomarkers. In particular embodiments, the multivariate biomarker panel includes 10 biomarkers. In particular embodiments, the multivariate biomarker panel includes 11 biomarkers. In particular embodiments, the multivariate biomarker panel includes 12 biomarkers. In particular embodiments, the multivariate biomarker panel includes 13 biomarkers. In particular embodiments, the multivariate biomarker panel includes 14 biomarkers. In particular embodiments, the multivariate biomarker panel includes 15 biomarkers. In particular embodiments, the multivariate biomarker panel includes 16 biomarkers. In particular embodiments, the multivariate biomarker panel includes 17 biomarkers. In particular embodiments, the multivariate biomarker panel includes 18 biomarkers. In particular embodiments, the multivariate biomarker panel includes 19 biomarkers. In particular embodiments, the multivariate biomarker panel includes 20 biomarkers. In particular embodiments, the multivariate biomarker panel includes 21 biomarkers. In particular embodiments, the multivariate biomarker panel includes 22 biomarkers. In particular embodiments, the multivariate biomarker panel includes 23 biomarkers. In particular embodiments, the multivariate biomarker panel includes 24 biomarkers. In particular embodiments, the multivariate biomarker panel includes 25 biomarkers. In particular embodiments, the multivariate biomarker panel includes 26 biomarkers. In particular embodiments, the multivariate biomarker panel includes 27 biomarkers. In particular embodiments, the multivariate biomarker panel includes 28 biomarkers. In particular embodiments, the multivariate biomarker panel includes 29 biomarkers. In particular embodiments, the multivariate biomarker panel includes 30 biomarkers. In particular embodiments, the multivariate biomarker panel includes 31 biomarkers. In particular embodiments, the multivariate biomarker panel includes 32 biomarkers. In particular embodiments, the multivariate biomarker panel includes 33 biomarkers. In particular embodiments, the multivariate biomarker panel includes 34 biomarkers. In particular embodiments, the multivariate biomarker panel includes 35 biomarkers. In particular embodiments, the multivariate biomarker panel includes 36 biomarkers. In particular embodiments, the multivariate biomarker panel includes 37 biomarkers. In particular embodiments, the multivariate biomarker panel includes 38 biomarkers. In particular embodiments, the multivariate biomarker panel includes 39 biomarkers. In particular embodiments, the multivariate biomarker panel includes 40 biomarkers. In particular embodiments, the multivariate biomarker panel includes 41 biomarkers. In particular embodiments, the multivariate biomarker panel includes 42 biomarkers. In particular embodiments, the multivariate biomarker panel includes 43 biomarkers. In particular embodiments, the multivariate biomarker panel includes 44 biomarkers. In particular embodiments, the multivariate biomarker panel includes 45 biomarkers. In particular embodiments, the multivariate biomarker panel includes 46 biomarkers. In particular embodiments, the multivariate biomarker panel includes 47 biomarkers. In particular embodiments, the multivariate biomarker panel includes 48 biomarkers. In particular embodiments, the multivariate biomarker panel includes 49 biomarkers. In particular embodiments, the multivariate biomarker panel includes 50 biomarkers. In particular embodiments, the multivariate biomarker panel includes 51 biomarkers. In particular embodiments, the multivariate biomarker panel includes 52 biomarkers. In particular embodiments, the multivariate biomarker panel includes 53 biomarkers. In particular embodiments, the multivariate biomarker panel includes 54 biomarkers. In particular embodiments, the multivariate biomarker panel includes 55 biomarkers. In particular embodiments, the multivariate biomarker panel includes 56 biomarkers. In particular embodiments, the multivariate biomarker panel includes 57 biomarkers. In particular embodiments, the multivariate biomarker panel includes 58 biomarkers. In particular embodiments, the multivariate biomarker panel includes 59 biomarkers. In particular embodiments, the multivariate biomarker panel includes 60 biomarkers. In particular embodiments, the multivariate biomarker panel includes 61 biomarkers. In particular embodiments, the multivariate biomarker panel includes 62 biomarkers. In particular embodiments, the multivariate biomarker panel includes 63 biomarkers. In particular embodiments, the multivariate biomarker panel includes 64 biomarkers. In particular embodiments, the multivariate biomarker panel includes 65 biomarkers. In particular embodiments, the multivariate biomarker panel includes 66 biomarkers. In particular embodiments, the multivariate biomarker panel includes 67 biomarkers. In particular embodiments, the multivariate biomarker panel includes 68 biomarkers. In particular embodiments, the multivariate biomarker panel includes 69 biomarkers. In particular embodiments, the multivariate biomarker panel includes 70 biomarkers. In particular embodiments, the multivariate biomarker panel includes 71 biomarkers. In particular embodiments, the multivariate biomarker panel includes 72 biomarkers. In particular embodiments, the multivariate biomarker panel includes 73 biomarkers. In particular embodiments, the multivariate biomarker panel includes 74 biomarkers. In particular embodiments, the multivariate biomarker panel includes 75 biomarkers. In particular embodiments, the multivariate biomarker panel includes 76 biomarkers. In particular embodiments, the multivariate biomarker panel includes 77 biomarkers. In particular embodiments, the multivariate biomarker panel includes 78 biomarkers. In particular embodiments, the multivariate biomarker panel includes 79 biomarkers. In particular embodiments, the multivariate biomarker panel includes 80 biomarkers. In particular embodiments, the multivariate biomarker panel includes 81 biomarkers. In particular embodiments, the multivariate biomarker panel includes 82 biomarkers. In particular embodiments, the multivariate biomarker panel includes 83 biomarkers. In particular embodiments, the multivariate biomarker panel includes 84 biomarkers. In particular embodiments, the multivariate biomarker panel includes 85 biomarkers. In particular embodiments, the multivariate biomarker panel includes 86 biomarkers. In particular embodiments, the multivariate biomarker panel includes 87 biomarkers. In particular embodiments, the multivariate biomarker panel includes 88 biomarkers. In particular embodiments, the multivariate biomarker panel includes 89 biomarkers. In particular embodiments, the multivariate biomarker panel includes 90 biomarkers. In particular embodiments, the multivariate biomarker panel includes 91 biomarkers. In particular embodiments, the multivariate biomarker panel includes 92 biomarkers. In particular embodiments, the multivariate biomarker panel includes 93 biomarkers. In particular embodiments, the multivariate biomarker panel includes 94 biomarkers. In particular embodiments, the multivariate biomarker panel includes 95 biomarkers. In particular embodiments, the multivariate biomarker panel includes 96 biomarkers. In particular embodiments, the multivariate biomarker panel includes 97 biomarkers. In particular embodiments, the multivariate biomarker panel includes 98 biomarkers. In particular embodiments, the multivariate biomarker panel includes 99 biomarkers.

In particular embodiments described herein, a biomarker panel is implemented for the assessment or prediction of disease progression, such as MS disease progression. In various embodiments, the assessment of disease progression involves implementing a univariate biomarker panel. Therefore, the univariate biomarker panel includes one biomarker. In other embodiments, the assessment of disease progression involves implementing a multivariate biomarker panel. In such embodiments, the multivariate biomarker panel for assessing disease progression includes more than one biomarker. In various embodiments, the multivariate biomarker panel for assessing disease progression includes two biomarkers. In various embodiments, the multivariate biomarker panel for assessing disease progression includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 biomarkers. In particular embodiments, the multivariate biomarker panel includes 2 biomarkers. In particular embodiments, the multivariate biomarker panel includes 3 biomarkers. In particular embodiments, the multivariate biomarker panel includes 4 biomarkers. In particular embodiments, the multivariate biomarker panel includes 5 biomarkers. In particular embodiments, the multivariate biomarker panel includes 6 biomarkers. In particular embodiments, the multivariate biomarker panel includes 7 biomarkers. In particular embodiments, the multivariate biomarker panel includes 8 biomarkers. In particular embodiments, the multivariate biomarker panel includes 9 biomarkers. In particular embodiments, the multivariate biomarker panel includes 10 biomarkers. In particular embodiments, the multivariate biomarker panel includes 11 biomarkers. In particular embodiments, the multivariate biomarker panel includes 12 biomarkers. In particular embodiments, the multivariate biomarker panel includes 13 biomarkers. In particular embodiments, the multivariate biomarker panel includes 14 biomarkers. In particular embodiments, the multivariate biomarker panel includes 15 biomarkers. In particular embodiments, the multivariate biomarker panel includes 16 biomarkers. In particular embodiments, the multivariate biomarker panel includes 17 biomarkers. In particular embodiments, the multivariate biomarker panel includes 18 biomarkers. In particular embodiments, the multivariate biomarker panel includes 19 biomarkers. In particular embodiments, the multivariate biomarker panel includes 20 biomarkers. In particular embodiments, the multivariate biomarker panel includes 21 biomarkers. In particular embodiments, the multivariate biomarker panel includes 22 biomarkers. In particular embodiments, the multivariate biomarker panel includes 23 biomarkers. In particular embodiments, the multivariate biomarker panel includes 24 biomarkers. In particular embodiments, the multivariate biomarker panel includes 25 biomarkers. In particular embodiments, the multivariate biomarker panel includes 26 biomarkers. In particular embodiments, the multivariate biomarker panel includes 27 biomarkers. In particular embodiments, the multivariate biomarker panel includes 28 biomarkers. In particular embodiments, the multivariate biomarker panel includes 29 biomarkers. In particular embodiments, the multivariate biomarker panel includes 30 biomarkers. In particular embodiments, the multivariate biomarker panel includes 31 biomarkers. In particular embodiments, the multivariate biomarker panel includes 32 biomarkers. In particular embodiments, the multivariate biomarker panel includes 33 biomarkers. In particular embodiments, the multivariate biomarker panel includes 34 biomarkers. In particular embodiments, the multivariate biomarker panel includes 35 biomarkers. In particular embodiments, the multivariate biomarker panel includes 36 biomarkers. In particular embodiments, the multivariate biomarker panel includes 37 biomarkers. In particular embodiments, the multivariate biomarker panel includes 38 biomarkers. In particular embodiments, the multivariate biomarker panel includes 39 biomarkers. In particular embodiments, the multivariate biomarker panel includes 40 biomarkers. In particular embodiments, the multivariate biomarker panel includes 41 biomarkers. In particular embodiments, the multivariate biomarker panel includes 42 biomarkers. In particular embodiments, the multivariate biomarker panel includes 43 biomarkers. In particular embodiments, the multivariate biomarker panel includes 44 biomarkers. In particular embodiments, the multivariate biomarker panel includes 45 biomarkers. In particular embodiments, the multivariate biomarker panel includes 46 biomarkers. In particular embodiments, the multivariate biomarker panel includes 47 biomarkers. In particular embodiments, the multivariate biomarker panel includes 48 biomarkers. In particular embodiments, the multivariate biomarker panel includes 49 biomarkers. In particular embodiments, the multivariate biomarker panel includes 50 biomarkers. In particular embodiments, the multivariate biomarker panel includes 51 biomarkers. In particular embodiments, the multivariate biomarker panel includes 52 biomarkers. In particular embodiments, the multivariate biomarker panel includes 53 biomarkers. In particular embodiments, the multivariate biomarker panel includes 54 biomarkers. In particular embodiments, the multivariate biomarker panel includes 55 biomarkers. In particular embodiments, the multivariate biomarker panel includes 56 biomarkers. In particular embodiments, the multivariate biomarker panel includes 57 biomarkers. In particular embodiments, the multivariate biomarker panel includes 58 biomarkers. In particular embodiments, the multivariate biomarker panel includes 59 biomarkers. In particular embodiments, the multivariate biomarker panel includes 60 biomarkers. In particular embodiments, the multivariate biomarker panel includes 61 biomarkers. In particular embodiments, the multivariate biomarker panel includes 62 biomarkers. In particular embodiments, the multivariate biomarker panel includes 63 biomarkers. In particular embodiments, the multivariate biomarker panel includes 64 biomarkers. In particular embodiments, the multivariate biomarker panel includes 65 biomarkers. In particular embodiments, the multivariate biomarker panel includes 66 biomarkers. In particular embodiments, the multivariate biomarker panel includes 67 biomarkers. In particular embodiments, the multivariate biomarker panel includes 68 biomarkers. In particular embodiments, the multivariate biomarker panel includes 69 biomarkers. In particular embodiments, the multivariate biomarker panel includes 70 biomarkers. In particular embodiments, the multivariate biomarker panel includes 71 biomarkers. In particular embodiments, the multivariate biomarker panel includes 72 biomarkers. In particular embodiments, the multivariate biomarker panel includes 73 biomarkers. In particular embodiments, the multivariate biomarker panel includes 74 biomarkers. In particular embodiments, the multivariate biomarker panel includes 75 biomarkers. In particular embodiments, the multivariate biomarker panel includes 76 biomarkers. In particular embodiments, the multivariate biomarker panel includes 77 biomarkers. In particular embodiments, the multivariate biomarker panel includes 78 biomarkers. In particular embodiments, the multivariate biomarker panel includes 79 biomarkers. In particular embodiments, the multivariate biomarker panel includes 80 biomarkers. In particular embodiments, the multivariate biomarker panel includes 81 biomarkers. In particular embodiments, the multivariate biomarker panel includes 82 biomarkers. In particular embodiments, the multivariate biomarker panel includes 83 biomarkers. In particular embodiments, the multivariate biomarker panel includes 84 biomarkers. In particular embodiments, the multivariate biomarker panel includes 85 biomarkers. In particular embodiments, the multivariate biomarker panel includes 86 biomarkers. In particular embodiments, the multivariate biomarker panel includes 87 biomarkers. In particular embodiments, the multivariate biomarker panel includes 88 biomarkers. In particular embodiments, the multivariate biomarker panel includes 89 biomarkers. In particular embodiments, the multivariate biomarker panel includes 90 biomarkers. In particular embodiments, the multivariate biomarker panel includes 91 biomarkers. In particular embodiments, the multivariate biomarker panel includes 92 biomarkers. In particular embodiments, the multivariate biomarker panel includes 93 biomarkers. In particular embodiments, the multivariate biomarker panel includes 94 biomarkers. In particular embodiments, the multivariate biomarker panel includes 95 biomarkers. In particular embodiments, the multivariate biomarker panel includes 96 biomarkers. In particular embodiments, the multivariate biomarker panel includes 97 biomarkers. In particular embodiments, the multivariate biomarker panel includes 98 biomarkers. In particular embodiments, the multivariate biomarker panel includes 99 biomarkers.

In various embodiments, a multivariate biomarker panel further incorporates one or more subject attributes. For example, subject attributes can include an age of the subject, the gender of the subject, a disease duration experienced by the subject (e.g., disease duration of MS), racial/ethnic identity, weight, height, body mass index (BMI), and socioeconomic status.

In an embodiment, the biomarkers in the biomarker panel can include one or more of: T-cell surface glycoprotein CD1c (CD1C), disks large homolog 4 (DLG4), thioredoxin domain containing 15 (TXNDC15), superoxide dismutase (SOD2), trem-like transcript 1 protein (TREML1), immunoglobulin superfamily DCC subclass member 4 (IGDCC4), lamin B2 (LMNB2), guanine nucleotide-binding protein G(s) subunit alpha isoforms short (GNAS), CXADR-like membrane protein (CLMP), glial fibrillary acidic protein (GFAP), neurofilament light polypeptide (NEFL), C—X—C motif chemokine 13 (CXCL-13), amyloid beta precursor like protein 1 (APLP1), myelin-oligodendrocyte glycoprotein (MOG), osteoprotegerin (OPG), versican core protein (VCAN), CUB domain containing protein 1 (CDCP1), tumor necrosis factor ligand superfamily member 13B (TNFSF13b), contactin 2 (CNTN2), C—X—C motif chemokine 9 (CXCL9), serpin family A member 9 (SERPINA9), osteopontin (OPN), T-cell differentiation antigen CD6 (CD6), tumor necrosis factor receptor superfamily member 10A (TNFRSF10a), C—C motif chemokine 20 (CCL20), (FLRT2), collagen type IV alpha 1 chain (COL4A1), growth Hormone (GH), interleukin 12 (IL-12), protogenin (PRTG), C—X—C motif chemokine 10 (CXCL10), interleukin 15 (IL15), pro-epidermal growth factor (EGF), C—X—C motif chemokine 11 (CXCL11), complement factor H (CFH), tumor necrosis factor ligand superfamily member 10 (TNFSF10), interleukin 18 (IL18), interleukin 6 (IL6), tumor necrosis factor (TNF), hepatitis A virus cellular receptor 1 (HAVCR1), receptor-type tyrosine-protein kinase FLT3 (FLT3), mannosyl-oligosaccharide 1,2-alpha-mannosidase IB (MAN1A2), aminoacylase 3 (ACY3), rho guanine nucleotide exchange factor 1 (ARHGEF1), adhesion G-protein coupled receptor G1 (ADGRG1), E3 ubiquitin-protein ligase MYCBP2 (MYCBP2), integrin beta-1 (ITGB1), C-type lectin domain family 4 member A (CLEC4A), meprin A subunit beta (MEP1B), coagulation factor XIII B chain (F13B), ficolin-1 (FCN1), adenylate cyclase activating polypeptide 1 (Pituitary) receptor type I (ADCYAP1R1), leukocyte immunoglobulin like receptor A5 (LILRA5), hephaestin (HEPH), C-type lectin domain family 10 member A (CLEC10A), rab9 effector protein with kelch motifs (RABEPK), low affinity immunoglobulin epsilon Fc receptor (FCER2), thyroglobulin (TG), stromal cell-derived factor 1 (CXCL12), carbonic anhydrase 3 (CA3), interleukin-8 (CXCL8), C—C motif chemokine 8 (CCL8), B-cell receptor CD22 (CD22), interleukin-17A (IL17A), interleukin-7 (IL7), kelch-like protein 41 (KLHL41), killer cell lectin-like receptor subfamily C member 1 (KLRC1), Fc receptor-like protein 1 (FCRL1), interleukin-17C (IL17C), plasma kallikrein (KLKB1), interferon gamma receptor 2 (IFNGR2), cystatin-F (CST7), fms-related tyrosine kinase 3 ligand (FLT3LG), C—C motif chemokine 19 (CCL19), GDNF family receptor alpha-2 (GFRA2), serpin family A member 3 (SERPINA3), kirre like nephrin family adhesion molecule 1 (KIRREL1), lymphotoxin-alpha (LTA), adenosine monophosphate deaminase 3 (AMPD3), C—C motif chemokine 2 (CCL2), dipeptidase 2 (DPEP2), complement factor H-related protein 5 (CFHR5), coagulation factor X (F10), serpin family D member 1 (SERPIND1), granulocyte colony-stimulating factor (CSF3), C—C motif chemokine 13 (CCL13), 6-phosphofructo-2-kinase/fructose-2,6-bisphosphatase 2 (PFKFB2), macrophage colony-stimulating factor 1 (CSF1), apolipoprotein L1 (APOF), macrophage metalloelastase (MMP12), leiomodin-1 (LMOD1), inactive ribonuclease-like protein 10 (RNASE10), serum amyloid P-component (APCS), interstitial collagenase (MMP1), centrosomal protein 20 (CEP20), nicotinamide phosphoribosyltransferase (NAMPT), oxidized low-density lipoprotein receptor 1 (OLR1), ADAMTS-like protein 2 (ADAMTSL2), and vascular endothelial growth factor A long form (VEGFA).

In some embodiments, the biomarkers in the biomarker panel include biomarkers shown in Tables 4-6. In some embodiments, the biomarkers can include one or more of: Neurofilament Light Polypeptide Chain (NEFL), Myelin Oligodendrocyte Glycoprotein (MOG), Cluster of Differentiation 6 (CD6), Chemokine (C—X—C motif) ligand 9 (CXCL9), Osteoprotegerin (OPG), Osteopontin (OPN), Matrix Metallopeptidase 9 (MMP-9), Glial Fibrillary Acidic Protein (GFAP), CUB domain-containing protein 1 (CDCP1), C—C Motif Chemokine Ligand 20 (CCL20/MIP 3-α), Interleukin-12 subunit beta (IL-12B), Amyloid Beta Precursor Like Protein 1 (APLP1), Tumor Necrosis Factor Receptor Superfamily Member 10A (TNFRSF10A), Collagen, type IV, alpha 1 (COL4A1), Serpin Family A Member 9 (SERPINA9), Fibronectin Leucine Rich Transmembrane Protein 2 (FLRT2), Chemokine (C—X—C motif) ligand 13 (CXCL13), Growth Hormone (GH), Versican core protein (VCAN), Protogenin (PRTG), Contactin-2 (CNTN2). In some embodiments, the biomarkers further include Growth Hormone (GH2), Interleukin-18 (IL18), Matrix Metalloproteinase-2 (MMP-2), Gamma-Interferon-Inducible Lysosomal Thiol Reductase (IFI30), and Chitinase-3-like protein 1 (CHI3L1/YkL40).

In some embodiments, the biomarkers can include one or more of: Cell Adhesion Molecule 3 (CADM3), Kallikrein Related Peptidase 6 (KLK6), Brevican (BCAN), Oligodendrocyte Myelin Glycoprotein (OMG), CD5 molecule (CD5), Cytotoxic and Regulatory T Cell Molecule (CRTAM), CD244 Molecule (CD244), Tumor Necrosis Factor Receptor Superfamily Member 9 (TNFRSF9), Proteinase 3 (PRTN3), Follistatin Like 3 (FSTL3), C—X—C Motif Chemokine Ligand 10 (CXCL10), C—X—C Motif Chemokine Ligand 11 (CXCL11), Interleukin 18 Binding Protein (IL-18BP), Macrophage Scavenger Receptor 1 (MSR1), C—C Motif Chemokine Ligand 3 (CCL3), Tumor Necrosis Factor Ligand Superfamily Member 12 (TWEAK), Trefoil Factor 3 (TFF3), Ectonucleotide Pyrophosphatase/Phosphodiesterase 2 (ENPP2), Insulin Like Growth Factor Binding Protein 1 (IGFBP-1), Interleukin 12A (IL12A), Seizure Related 6 Homolog Like (SEZ6L), Dipeptidyl Peptidase Like 6 (DPP6), Neurocan (NCAN), Tubulointerstitial Nephritis Antigen Like 1 (TINAGL1), Calcium Activated Nucleotidase 1 (CANT1), Nectin Cell Adhesion Molecule 2 (NECTIN2), Neural Proliferation, Differentiation and Control Protein 1 (NPDC1), Tumor Necrosis Factor Receptor Superfamily Member 11A (TNFRSF11A), Contactin 4 (CNTN4), Neutrophic Receptor Tyrosine Kinase 2 (NTRK2), Neutrophic Receptor Tyrosine Kinase 3 (NTRK3), Cadherin 6 (CDH6), Carcinoembryonic Antigen Related Cell Adhesion Molecule 8 (CEACAM8), Mitotic Arrest Deficient 1 Like 1 (MAD1L1), Fc Fragment of IgA Receptor (FCAR), Myeloperoxidase (MPO), Osteomodulin (OMD), Matrix Extracellular Phosphoglycoprotein (MEPE), GDNF Family Receptor Alpha 3 (GDNFR-alpha-3), Scavenger Receptor Class F Member 2 (SCARF2), CD40 Ligand (IgM), Tumor Necrosis Factor Receptor Superfamily Member 1B (TNF-R2), Programmed Cell Death 1 Ligand (PD-L1), Notch 3 (NOTCH3), Contactin 1 (CNTN1), Oncostatin M (OSM), Transforming Growth Factor Alpha (TGF-α), Peptidoglycan Recognition Protein 1 (PGLYRP1), Nitric Oxide Synthase 3 (NOS3).

In various embodiments, the biomarker panel useful for generating a prediction (e.g., a prediction for MS disease activity or a prediction for MS disease progression) comprises at least one, at least two, at least three, at least four, at least five, or more biomarkers selected from: CD1C, DLG4, TXNDC15, SOD2, TREML1, IGDCC4, LMNB2, GNAS, CLMP, GFRA2, ARHGEF1, FLRT2, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, IL15, GFAP, APLP1, MOG, OPG, VCAN, CDCP1, TNFSF13b, CNTN2, CXCL9, SERPINA9, OPN, CD6, TNFRSF10a, CCL20, COL4A1, GH, IL-12, PRTG, CXCL10, EGF, CXCL11, CFH, TNFSF10, IL18, IL6, TNF, CXCL13, NEFL, CCL20/MIP 3-α, and FLRT2.

In particular embodiments, the biomarker panel useful for generating a prediction (e.g., a prediction for MS disease activity or a prediction for MS disease progression) includes biomarkers identified as Tier 1 in Table 1, Tier 2 in Table 2, or Tier 3 in Table 3. For example, the biomarker panel includes CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL (Tier 1 in Table 1). As another example, the biomarker panel includes CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2 (Tier 2 in Table 2). As another example, the biomarker panel includes CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL (Tier 3 in Table 3).

In various embodiments, the biomarker panel for generating a prediction (e.g., a prediction for disease activity or a prediction for disease progression) includes a minimal set of predictive biomarkers, such as a set of at least 2 biomarkers. In various embodiments, at least one of the biomarkers in a set of at least 2 biomarkers is NEFL. In various embodiments, at least one of the biomarkers in a set of at least 2 biomarkers is GFAP. In various embodiments, at least one of the biomarkers in a set of at least 2 biomarkers is CD1C. In various embodiments, at least one of the biomarkers in a set of at least 2 biomarkers is DLG4. In various embodiments, at least one of the biomarkers in a set of at least 2 biomarkers is TXNDC15. In various embodiments, at least one of the biomarkers in a set of at least 2 biomarkers is SOD2. In various embodiments, at least one of the biomarkers in a set of at least 2 biomarkers is TREML1. In various embodiments, at least one of the biomarkers in a set of at least 2 biomarkers is IGDCC4. In various embodiments, at least one of the biomarkers in a set of at least 2 biomarkers is GNAS. In various embodiments, at least one of the biomarkers in a set of at least 2 biomarkers is LMNB2. In various embodiments, at least one of the biomarkers in a set of at least 2 biomarkers is CLMP. In various embodiments, at least one of the biomarkers in a set of at least 2 biomarkers is GFRA2. In various embodiments, at least one of the biomarkers in a set of at least 2 biomarkers is HAVCR1. In various embodiments, at least one of the biomarkers in a set of at least 2 biomarkers is FLT3. In various embodiments, at least one of the biomarkers in a set of at least 2 biomarkers is MEP1B. In various embodiments, at least one of the biomarkers in a set of at least 2 biomarkers is F13B. In various embodiments, at least one of the biomarkers in a set of at least 2 biomarkers is IL15.

In various embodiments, the biomarker panel comprises one or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL. In various embodiments, the biomarker panel comprises at least one biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL. In various embodiments, the biomarker panel comprises a set of biomarkers of GFRA2, and GFAP. In various embodiments, the biomarker panel comprises a set of biomarkers of GFRA2, GFAP, and TREML1. In various embodiments, the biomarker panel comprises a set of biomarkers of GFRA2, GFAP, TREML1, and CLMP. In various embodiments, the biomarker panel comprises a set of biomarkers of GFRA2, GFAP, TREML1, CLMP, and SOD2. In various embodiments, the biomarker panel comprises a set of biomarkers of GFRA2, GFAP, TREML1, CLMP, SOD2, and IGDCC4. In various embodiments, the biomarker panel comprises a set of biomarkers of GFRA2, GFAP, TREML1, CLMP, SOD2, IGDCC4, and GNAS. In various embodiments, the biomarker panel comprises a set of biomarkers of GFRA2, GFAP, TREML1, CLMP, SOD2, IGDCC4, GNAS, and NEFL. In various embodiments, the biomarker panel comprises a set of biomarkers of GFRA2, GFAP, TREML1, CLMP, SOD2, IGDCC4, GNAS, NEFL, and DLG4. In various embodiments, the biomarker panel comprises a set of biomarkers of GFRA2, GFAP, TREML1, CLMP, SOD2, IGDCC4, GNAS, NEFL, DLG4, and TXNDC15. In various embodiments, the biomarker panel comprises a set of biomarkers of GFRA2, GFAP, TREML1, CLMP, SOD2, IGDCC4, GNAS, NEFL, DLG4, TXNDC15, and CD1C. In various embodiments, the biomarker panel comprises a set of biomarkers of GFRA2, GFAP, TREML1, CLMP, SOD2, IGDCC4, GNAS, NEFL, DLG4, TXNDC15, CD1C, and IL15. In various embodiments, the biomarker panel comprises a set of biomarkers of GFRA2, GFAP, TREML1, CLMP, SOD2, IGDCC4, GNAS, NEFL, DLG4, TXNDC15, CD1C, IL15, and FLT3. In various embodiments, the biomarker panel comprises a set of biomarkers of GFRA2, GFAP, TREML1, CLMP, SOD2, IGDCC4, GNAS, NEFL, DLG4, TXNDC15, CD1C, IL15, FLT3, and MEP1B. In various embodiments, the biomarker panel comprises a set of biomarkers of GFRA2, GFAP, TREML1, CLMP, SOD2, IGDCC4, GNAS, NEFL, DLG4, TXNDC15, CD1C, IL15, FLT3, MEP1B, and HAVCR1. In various embodiments, the biomarker panel comprises a set of biomarkers of GFRA2, GFAP, TREML1, CLMP, SOD2, IGDCC4, GNAS, NEFL, DLG4, TXNDC15, CD1C, IL15, FLT3, MEP1B, HAVCR1, and F13B.

In various embodiments, the biomarker panel comprises one or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2. In various embodiments, the biomarker panel comprises at least one biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2. In some embodiments, the biomarker panel comprises a set of TNFSF13B, and GNAS. In some embodiments, the biomarker panel comprises a set of TNFSF13B, GNAS, and NEFL. In some embodiments, the biomarker panel comprises a set of TNFSF13B, GNAS, NEFL, and GFAP. In some embodiments, the biomarker panel comprises a set of TNFSF13B, GNAS, NEFL, GFAP, and GFRA2. In some embodiments, the biomarker panel comprises a set of TNFSF13B, GNAS, NEFL, GFAP, GFRA2, and IGDCC4. In some embodiments, the biomarker panel comprises a set of TNFSF13B, GNAS, NEFL, GFAP, GFRA2, IGDCC4, and CLMP. In some embodiments, the biomarker panel comprises a set of TNFSF13B, GNAS, NEFL, GFAP, GFRA2, IGDCC4, CLMP, and DLG4. In some embodiments, the biomarker panel comprises a set of TNFSF13B, GNAS, NEFL, GFAP, GFRA2, IGDCC4, CLMP, DLG4, and SERPINA9. In some embodiments, the biomarker panel comprises a set of TNFSF13B, GNAS, NEFL, GFAP, GFRA2, IGDCC4, CLMP, DLG4, SERPINA9, and OPG. In some embodiments, the biomarker panel comprises a set of TNFSF13B, GNAS, NEFL, GFAP, GFRA2, IGDCC4, CLMP, DLG4, SERPINA9, OPG, and SOD2. In some embodiments, the biomarker panel comprises a set of TNFSF13B, GNAS, NEFL, GFAP, GFRA2, IGDCC4, CLMP, DLG4, SERPINA9, OPG, SOD2, and CXCL13. In some embodiments, the biomarker panel comprises a set of TNFSF13B, GNAS, NEFL, GFAP, GFRA2, IGDCC4, CLMP, DLG4, SERPINA9, OPG, SOD2, CXCL13, and CD6. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, and CDCP1. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, and OPN. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, and PRTG. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, and CCL20. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, and FLT3. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, and CNTN2. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, and TXNDC15. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, and IL15. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, and IL12B. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, and APLP1. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, and FLRT2. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, and CD1C. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, and TREML1. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, TREML1, and MEP1B. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, TREML1, MEP1B, and TNFRSF10A. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, TREML1, MEP1B, TNFRSF10A, and MOG. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, TREML1, MEP1B, TNFRSF10A, MOG, and CXCL9. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, TREML1, MEP1B, TNFRSF10A, MOG, CXCL9, and HAVCR1. In some embodiments, the biomarker panel comprises a set of TNFSF13B, GNAS, NEFL, GFAP, GFRA2, IGDCC4, CLMP, DLG4, SERPINA9, OPG, SOD2, CXCL13, CD6, CDCP1, OPN, PRTG, CCL20, FLT3, CNTN2, TXNDC15, IL15, IL12B, APLP1, FLRT2, CD1C, TREML1, MEP1B, TNFRSF10A, MOG, CXCL9, HAVCR1, and F13B.

In various embodiments, the biomarker panel comprises one or more biomarkers selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL. In various embodiments, the biomarker panel comprises at least one biomarkers selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL. In some embodiments, the biomarker panel comprises a set of SERPIND1, and KLRC1. In some embodiments, the biomarker panel comprises a set of SERPIND1, KLRC1, and CLMP. In some embodiments, the biomarker panel comprises a set of SERPIND1, KLRC1, CLMP, and GFRA2. In some embodiments, the biomarker panel comprises a set of SERPIND1, KLRC1, CLMP, GFRA2, and GFAP. In some embodiments, the biomarker panel comprises a set of SERPIND1, KLRC1, CLMP, GFRA2, GFAP, and TG. In some embodiments, the biomarker panel comprises a set of SERPIND1, KLRC1, CLMP, GFRA2, GFAP, TG, and IL17A. In some embodiments, the biomarker panel comprises a set of SERPIND1, KLRC1, CLMP, GFRA2, GFAP, TG, IL17A, and OLR1. In some embodiments, the biomarker panel comprises a set of SERPIND1, KLRC1, CLMP, GFRA2, GFAP, TG, IL17A, OLR1, and CA3. In some embodiments, the biomarker panel comprises a set of SERPIND1, KLRC1, CLMP, GFRA2, GFAP, TG, IL17A, OLR1, CA3, and SERPINA3. In some embodiments, the biomarker panel comprises a set of SERPIND1, KLRC1, CLMP, GFRA2, GFAP, TG, IL17A, OLR1, CA3, SERPINA3, and GNAS. In some embodiments, the biomarker panel comprises a set of SERPIND1, KLRC1, CLMP, GFRA2, GFAP, TG, IL17A, OLR1, CA3, SERPINA3, GNAS, and KLHL41. In some embodiments, the biomarker panel comprises a set of SERPIND1, KLRC1, CLMP, GFRA2, GFAP, TG, IL17A, OLR1, CA3, SERPINA3, GNAS, KLHL41, and IFNGR2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, and ADGRG1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, and CXCL8. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, and APOF. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, and MMP12. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, and NEFL. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, and SOD2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, and CXCL12. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, and DPEP2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, and FCRL2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, and FLT3LG. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, and ITGB1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, and ARHGEF1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, and KIRREL1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, and AMPD3. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, and CFHR5. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, and F10. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, and CCL13. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, and CSF1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, and PFKFB2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, and IGDCC4. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, and TREML1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, and RNASE10. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, and MMP1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, and CEP20. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, and NAMPT. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, and VEGFA. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, and TXNDC15. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, and CST7. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, and CD1C. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, and ACY3. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, and FCRL1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, and IL15. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, and IL7. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, and DLG4. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, and CD22. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, and CCL8. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, and HEPH. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, and MYCBP2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, and FCN1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, and IL17C. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, and CCL19. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, and F13B. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, and LTA. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, and CSF3. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, and MEP1B. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, and KLKB1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, and FLT3. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, and ADCYAP1R1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, and CCL2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, and CLEC10A. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, and LMOD1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, and CLEC4A. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, and MAN1A2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, MAN1A2, and ADAMTSL2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, MAN1A2, ADAMTSL2, and FCER2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, MAN1A2, ADAMTSL2, FCER2, and LMNB2. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, MAN1A2, ADAMTSL2, FCER2, LMNB2, and RABEPK. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, MAN1A2, ADAMTSL2, FCER2, LMNB2, RABEPK, and HAVCR1. In some embodiments, the biomarker panel comprises a set of SERPIND1, KLRC1, CLMP, GFRA2, GFAP, TG, IL17A, OLR1, CA3, SERPINA3, GNAS, KLHL41, IFNGR2, ADGRG1, CXCL8, APOF, MMP12, NEFL, SOD2, CXCL12, DPEP2, FCRL2, FLT3LG, ITGB1, ARHGEF1, KIRREL1, AMPD3, CFHR5, F10, CCL13, CSF1, PFKFB2, IGDCC4, TREML1, RNASE10, MMP1, CEP20, NAMPT, VEGFA, TXNDC15, CST7, CD1C, ACY3, FCRL1, IL15, IL7, DLG4, CD22, CCL8, HEPH, MYCBP2, FCN1, IL17C, CCL19, F13B, LTA, CSF3, MEP1B, KLKB1, FLT3, ADCYAP1R1, CCL2, CLEC10A, LMOD1, CLEC4A, MAN1A2, ADAMTSL2, FCER2, LMNB2, RABEPK, HAVCR1, and LILRA5.

In various embodiments, the biomarker panel further comprises at least one biomarker selected from VCAN, COL4A1, GH, CXCL10, EGF, CXCL11, CFH, TNFSF10, IL18, IL6, TNF, CXCL13, and CCL20/MIP 3-α. In various embodiments, the biomarker panel further comprises VCAN. In various embodiments, the biomarker panel further comprises COL4A1. In various embodiments, the biomarker panel further comprises GH. In various embodiments, the biomarker panel further comprises CXCL10. In various embodiments, the biomarker panel further comprises EGF. In various embodiments, the biomarker panel further comprises CXCL11. In various embodiments, the biomarker panel further comprises CFH. In various embodiments, the biomarker panel further comprises TNFSF10. In various embodiments, the biomarker panel further comprises IL18. In various embodiments, the biomarker panel further comprises IL6. In various embodiments, the biomarker panel further comprises TNF. In various embodiments, the biomarker panel further comprises CXCL13. In various embodiments, the biomarker panel further comprises CCL20/MIP 3-α.

### V. Biomarkers

The dysregulation of biomarkers disclosed herein may contribute to the development and/or progression of disease activity, such as disease activity and/or disease progression of a neurodegenerative disease including multiple sclerosis, Parkinson's Disease, Lewy body disease, Alzheimer's Disease, Amyotrophic lateral sclerosis (ALS), motor neuron disease, Huntington's Disease, Spinal muscular atrophy, Friedreich's ataxia, Batten disease, and the like. Biomarkers, and the corresponding categorization of the biomarkers, are shown below in Table 7. Example categories include: neurodegeneration, myelin integrity, neuroaxonal integrity, cerebrovascular function, neurite outgrowth and neurogenesis, inflammation, neuroinflammation, immune modulation, cell regulation, cell adhesion, gut-brain axis, metabolism, and neuroregulatory categories. Exemplary biomarker categorizations are shown in FIG. 1D. Additionally, biomarkers and their involvement in particular locations (e.g., brain, brain barrier, or blood) and cell types are shown in Tables 8, 9A, and 9B.

NEFL is a 68 kDa biomarker that reflects axonal damage in the microenvironment. In other words, NEFL often serves as a proxy for axonal degeneration. Additionally, NEFL interacts with other biomarkers such as MAP2, Protein Kinase N1, and Tuberous sclerosis (TSC1).

COL4A1 is a 26 kDa biomarker involved in cell proliferation, migration, extracellular matrix formation, as well as inhibition of endothelial cell proliferation, migration, and tube formation. COL4A1 is involved in the outgrowth of hippocampal embryonic neurons and is further involved in myelin integrity. Type IV collagen is a major structural component of glomerular basement membranes (GBM), forming a chicken-wire mesh work together with laminins, proteoglycans and entactin/nidogen. It comprises a C-terminal NC1 domain, which inhibits angiogenesis and tumor formation. The C-terminal half is found to possess the anti-angiogenic activity. Type IV collage also inhibits endothelial cell proliferation, migration and tube formation as well as also inhibiting expression of hypoxia-inducible factor 1alpha and ERK1/2 and p38 MAPK activation. COL4A1 mutations are associated with a wide range of phenotypes that include both ischemic and hemorrhagic strokes, migraines, leukomalacia, nephropathy, hematuria, chronic muscle cramps, and ocular anterior segment diseases including congenital cataracts, glaucoma, and Axenfeld-Rieger anomalies. Case Rep Neurol. 2015 May-August; 7(2): 142-147. Published online 2015 Jun. 2. doi:10.1159/000431309.

APLP1 is a 72 kDa biomarker involved in synaptic maturation during cortical development and regulation of neurite outgrowth. APLP1 is one of two homologs: amyloid-like proteins 1 and 2, or APLP1 and APLP2. The encoding gene of APLP1 is a member of the highly conserved amyloid precursor protein gene family. The encoded protein is a membrane-associated glycoprotein that is cleaved by secretases in a manner similar to amyloid beta A4 precursor protein cleavage. This cleavage liberates an intracellular cytoplasmic fragment that may act as a transcriptional activator. APLP1 may also play a role in synaptic maturation during cortical development. Can regulate neurite outgrowth through binding to components of the extracellular matrix such as heparin and collagen I. APLP1 is extensively expressed in humans. Functions attributed to APLP1 include neurite outgrowth and synaptogenesis, protein trafficking along axons, cell adhesion, calcium metabolism, neuronal damage, synaptic dysfunction, and signal transduction.

MMP1 (Matrix Metalloproteinase-1) and MMP12 (Matrix Metalloproteinase-12) are enzymes belonging to the matrix metalloproteinase (MMP) family, which are responsible for the degradation of extracellular matrix proteins. These MMPs play pivotal roles in various physiological processes, including tissue remodeling, wound healing, and embryonic development, and are implicated in pathological conditions like inflammation, fibrosis, and cancer. MMP1, often referred to as interstitial collagenase, primarily degrades interstitial collagens, whereas MMP12, also known as macrophage elastase, primarily breaks down elastin but can degrade other extracellular matrix components as well.

FLRT2 is a 74 kDa biomarker and is a member of the fibronectin leucine rich transmembrane protein family, which function in cell adhesion and/or receptor signaling. FLRT2 is expressed in brain as well as in the heart and several other organs, and is involved in fibroblast growth factor-mediated signaling cascades. In the heart, it is required for normal organization of the cardiac basement membrane during embryogenesis, and for normal embryonic epicardium and heart morphogenesis. In the neurology context, FLRT2 functions in cell-cell adhesion, cell migration and axon guidance. It may play a role in the migration of cortical neurons during brain development via its interaction with UNC5D. FLRT2 is also involved in glutamate excitotoxicity, neuronal cell death, and synaptic formation & plasticity.

VCAN (>200 kDa biomarker) is involved in cell motility, cell growth and differentiation, cell adhesion, cell proliferation, cell migration, and angiogenesis. VCAN is further involved in myelin protection, astrocytic excitotoxicity, and is a proinflammatory mediator secretion. VCAN is a key factor in inflammation through interactions with adhesion molecules on the surfaces of inflammatory leukocytes and interactions with chemokines that are involved in recruiting inflammatory cells. In the adult central nervous system, versican is found in perineuronal nets, where it may stabilize synaptic connections. Versican can also inhibit nervous system regeneration and axonal growth following an injury to the central nervous system.

TNFSF13B, also herein referred to as B-cell activating factor (BAFF), is a biomarker involved in T cell-independent B cell activation and ectopic lymphoid follicle formation.

Interleukin-12 (IL-12) is a cytokine produced primarily by antigen-presenting cells such as dendritic cells and macrophages. It plays a critical role in regulating the immune response against intracellular pathogens. IL-12 promotes the differentiation of naive T cells into Th1 cells, which then produce interferon-gamma (IFN-γ) to further enhance the macrophage's ability to destroy intracellular pathogens. Additionally, IL-12 activates natural killer (NK) cells to produce IFN-7 and enhances their cytotoxic function. Through these mechanisms, IL-12 bridges the innate and adaptive immune responses and is vital for defending the host against certain bacterial, viral, and parasitic infections.

SERPINA9 is a 42 kDa biomarker that is a member of the serpin family of serine protease inhibitors. SERPINA9 is involved in neuronal damage. The expression of SERPINA9 is likely restricted to germinal center B cells and lymphoid malignancies. SERPINA9 is likely to function in vivo in the germinal center as an efficient inhibitor of trypsin-like proteases.

IL18 is involved in immune response and inflammatory processes. IL18 is a proinflammatory cytokine primarily involved in polarized T-helper 1 (Th1) cell and natural killer (NK) cell immune responses. It serves as an inhibitor of the early Th1 cytokine response. It further plays a role in Th-1 response through its ability to induce IFN-gamma production in T cells and NK cells. IL-18 in CSF and serum were significantly higher in comparison with the levels found in patients without enhancing lesions. The results suggest involvement of IL-18 in immunopathogenesis of MS especially in the active stages of the disease. Losy, J., et al. IL-18 in patients with multiple sclerosis. Acta Neurologica Scandinavica, 104:171-173 (2001). Additionally, higher IL-18 serum levels and significant different frequencies of two polymorphisms of IL-18 were found in MS patients. Jahanbani-Ardakani, H. et al., Interleukin 18 polymorphisms and its serum level in patients with multiple sclerosis, Ann Indian Acad Neurol; 22:474-76 (2019).

CDCP1 is a 90-140 kDa biomarker involved in T-cell migration, cell adhesion, and cell matrix association. CDCP1 may play a role in the regulation of anchorage versus migration or proliferation versus differentiation via its phosphorylation. CDCP1 is expressed in cells with phenotypes reminiscent of mesenchymal stem cells and neural stem cells. Additionally, CDCP1 is a ligand for CD6, a receptor molecule expressed on certain T-cells and may play a role in their migration and chemotaxis.

CNTN2 is a 113 kDa biomarker involved in cell adhesion, proliferation, migration, axon guidance of neurons, neuronal damage, and axon-dendritic rearrangement. CNTN2 is a member of the contactin family of proteins, part of the immunoglobulin superfamily of cell adhesion molecules. CNTN2 is a glycosylphosphatidylinositol (GPI)-anchored neuronal membrane protein and plays a role in the proliferation, migration, and axon guidance of neurons of the developing cerebellum. A mutation in CNTN2 gene may be associated with adult myoclonic epilepsy. In conjunction with another transmembrane protein, CNTNAP2, which contributes to the organization of axonal domains at nodes of Ranvier by maintaining voltage-gated potassium channels at the juxtaparanodal region.

GFAP is a 50 kDa biomarker involved in demyelination, degeneration, and neuroaxonal injury. Astroglial activation is associated with activation of the immune cascade and is thought to play a role in the demyelination and neuroaxonal injury observed in MS. Glial fibrillar acidic protein (GFAP) is the major constituent of gliotic scarring. GFAP is used as a marker to distinguish astrocytes from other glial cells during development. Higher serum concentrations of both GFAP and NEFL were associated with higher EDSS, older age, longer disease duration, progressive disease course and MRI pathology. Högel, H., et al., Serum glial fibrillary acidic protein correlates with multiple sclerosis disease severity. Multiple Sclerosis Journal, 26(13) 2018.

MOG is a 28 kDa membrane protein expressed on the oligodendrocyte cell surface and the outermost surface of myelin sheaths. Due to this localization, it serves as a cell surface receptor or cell adhesion molecule and is a primary target antigen involved in immune-mediated demyelination. This protein may be involved in completion and maintenance of the myelin sheath and in cell-cell communication. Diseases associated with MOG include Narcolepsy and Rubella. Among its related pathways are Neural Stem Cell Differentiation Pathways and Lineage-specific Markers. A paralog of the MOG gene is BTN1A1.

CD6 is a 90-130 kDa biomarker involved in central nervous system development. CD6 is a cell-adhesion molecule involved in blood brain barrier breach and T-cell mediated acute inflammatory response. Recent studies have identified CD6 as a risk gene for multiple sclerosis (MS), a disease in which autoreactive T cells are integrally involved. De Jager, P L., et al., Meta-analysis of genome scans and replication identify CD6, IRF8 and TNFRSF1A as new multiple sclerosis susceptibility loci. Nat Genet. 2009; 41(7):776-782. CD6 is found on the outer membrane of T-lymphocytes and is involved in the transmigration of leukocytes across the blood-brain barrier.

CXCL9 is a 12 kDa biomarker involved in immune response and inflammatory processes. CXCL9 is a cytokine that affects the growth, movement, or activation state of cells that participate in immune and inflammatory response. CXCL9 (MIG) is a chemokine that upon binding to its receptor CXCR3 elicits chemotactic activity on T cells and is involved in inflammatory response. CXCL9 is not constitutively expressed but is inducible by IFN-gamma. CXCL9 has been described to be involved in several inflammation-related diseases such as hepatitis C, skin inflammation, rheumatoid arthritis, and pharyngitis. Consistent with this observation is the upregulation of ELR-CXC chemokines, CXCL9, CXCL10 and CXCL11, which are upregulated in the CNS of EAE-affected mice induced by transfer of Th1 cells. Lovett-Racke, A., et al. Th1 versus Th17: Are T cell cytokines relevant in multiple sclerosis? Biochimca et Biophysica Acta (BBA)—Molecular Basis of Disease. 1812(2): 246-251 (2011).

CXCL13 is a biomarker involved in cell growth, cell reproduction, regeneration and inflammatory responses. CXCL13 belongs to the CXC chemokine family and is selectively chemotactic for B cells. It interacts with chemokine receptor CXCR5 through which it regulates the organization of B cells. Serum levels of CXCL13 have been implicated in multiple sclerosis. Festa, E. et al. Serum levels of CXCL13 are elevated in active multiple sclerosis. Multiple Sclerosis Journal, 15(11): 1271-1279 (2009).

CCL20 is a 11 kDa biomarker involved in axonal guidance and chemotaxis of dendritic cells. CCL20 is a chemokine involved in immunoregulatory and inflammatory processes (e.g., acute inflammatory response) and is expressed in epithelial cells of choroid plexus in the human brain. It serves as a cognate ligand of CCR6.

OPG is a 55-60 kDa biomarker involved in inflammation, cell apoptosis, and T-cell activation processes. OPG is a decoy receptor of cytokines TNFSF11 (RANKL) and possibly TNFSF10 (TRAIL) and belongs to the TNF receptor superfamily. OPG is up-regulated by estrogens and increasing calcium concentrations, and it has a role in transcriptional regulation in inflammation, innate immunity, and cell survival and differentiation; for example, OPG binding to TNFSF11 inhibits the differentiation of osteoclast precursors into mature osteoclasts and OPG has been used experimentally for the treatment of osteoporosis. OPG has been described to be involved in several inflammation-related diseases such as rheumatoid arthritis, inflammatory bowel disease, and periodontitis.

OPN is a 33-44 kDa biomarker involved in inflammation and immune modulation. OPN is a pleiotropic integrin binding protein with functions in cell-mediated immunity, inflammation, tissue repair, and cell survival. OPN also plays a role in biomineralization.

PRTG is a 180 kDa biomarker involved in neurogenesis, neurotrophin binding, neuronal survival, and demyelination. It may play a role in anteroposterior axis elongation. PRTG is a membrane protein and member of the immunoglobulin superfamily. It is considered to be primarily a developmental protein that has some associations to neuralgia, demyelinating diseases and dyslexia.

TNFRSF10A is a 50 kDa biomarker that is a member of the TNF-receptor superfamily. TNFRSF10A is involved in inflammation and neurodegenerative processes. This receptor is activated by tumor necrosis factor-related apoptosis inducing ligand (TNFSF10/TRAIL), and thus transduces cell death signal and induces cell apoptosis.

GH, also known as somatotropin or somatropin, is a neuroendocrine marker that stimulates growth, cell reproduction and regeneration in humans and other animals. It regulates energy homeostasis and metabolism. It is a type of mitogen which is specific only to certain kinds of cells. Prior studies have shown that it is decreased in the serum of severe MS patients. Gironi, M., et al. Growth hormone and Disease Severity in Early Stage of Multiple Sclerosis. Multiple Sclerosis International 2013: (2013).

CXCL8, CXCL9, CXCL10, CXCL11, CXCL12, and CXCL13 are each cytokines in the CXC chemokine family. CXCL8, CXCL9, CXCL10, CXCL11, CXCL12, and CXCL13 are involved in the biological processes of immune response, inflammatory response, cell signaling, chemotaxis, T-cell recruitment, and cell proliferation.

IL6 is a cytokine involved in differentiation of B-cells, lymphocytes, and monocytes.

CD1c is a member of the CD1 family of transmembrane glycoproteins, which are structurally related to the major histocompatibility complex (MHC) molecules. The size of CD1c is approximately 49 kDa. Unlike classical MHC molecules that present peptide antigens to T cells, CD1c molecules specialize in presenting lipid and glycolipid antigens to T cells. CD1c is primarily expressed on dendritic cells, B cells, and certain subsets of T cells. By presenting lipid antigens to T cells, CD1c plays a crucial role in mediating immune responses against lipid-rich pathogens, such as Mycobacterium tuberculosis, and may also be involved in immune responses to self-lipids, potentially playing a role in autoimmune conditions.

DLG4, also known as PSD-95 (Postsynaptic Density-95), is a member of the membrane-associated guanylate kinase (MAGUK) family of proteins. With a protein size of approximately 95 kDa, DLG4 primarily localizes to the postsynaptic density of excitatory synapses in the central nervous system. Functionally, DLG4 plays a pivotal role in synaptic signaling and plasticity. It acts as a scaffolding protein, clustering and anchoring various receptors, channels, and signaling molecules at synapses. This clustering is vital for effective synaptic transmission and the modulation of synaptic strength. Alterations in DLG4 expression or function have been implicated in various neurological disorders, including intellectual disabilities and neuropsychiatric diseases.

TXNDC15 (Thioredoxin Domain-Containing Protein 15) belongs to the thioredoxin protein family, which is known to play roles in redox signaling and other cellular processes. The size of the TXNDC15 is around 32 kDa.

SOD2, or manganese superoxide dismutase (MnSOD), is a 25 kDa antioxidant enzyme located within the mitochondria. SOD2 plays a critical role in protecting cells from oxidative damage by converting the superoxide radical (O2−) into hydrogen peroxide and molecular oxygen. By detoxifying reactive oxygen species (ROS) within mitochondria, SOD2 maintains cellular health and prevents damage that can lead to various pathological conditions, including neurodegenerative diseases, cardiovascular diseases, and cancer.

TREML1 is a cell surface receptor primarily expressed on platelets and myeloid cells. The size of TREML1 is roughly 30-35 kDa.

IGDCC4 is a member of the immunoglobulin superfamily.

LMNB2 is one of the lamin proteins, forming the nuclear lamina's structural framework adjacent to the inner nuclear membrane. The size of LMNB2 is approximately 67 kDa. Lamins, including LMNB2, are crucial for nuclear architecture, chromatin organization, DNA replication, and cell division. Mutations in lamin genes can lead to a variety of diseases known as laminopathies, which include neurological disorders and premature aging syndromes.

GNAS encodes the alpha subunit of the stimulatory G protein, which is involved in transmitting signals from various receptors to downstream effectors in cells. The approximate size is 45-52 kDa. GNAS plays a vital role in numerous signaling pathways, including those activated by hormones like adrenaline. Mutations in GNAS can result in several diseases, including Albright hereditary osteodystrophy and pseudohypoparathyroidism.

CLMP is a tight junction protein with an approximate size of 52 kDa. It's involved in the formation and maintenance of tight junctions between epithelial cells, ensuring proper paracellular barrier function. CLMP is essential for intestinal and renal epithelial tight junction formation and function. Mutations in the CLMP gene can lead to congenital short bowel syndrome, a severe and rare digestive disorder.

GFRA2 is a receptor for the glial cell line-derived neurotrophic factor (GDNF) family of ligands. The size of GFRA2 is approximately 48 kDa. It plays a crucial role in neuronal survival, differentiation, and repair, particularly in the peripheral nervous system. Binding of the ligand to GFRA2 activates the RET tyrosine kinase, initiating intracellular signaling pathways that promote neuronal survival and differentiation. GFRA2's function is vital for the development and maintenance of specific neuron populations.

### ARHGEF1 (Rho Guanine Nucleotide Exchange Factor 1)

ARHGEF1 is a member of the Dbl family of guanine nucleotide exchange factors (GEFs) for Rho GTPases. The protein size of ARHGEF1 is around 127 kDa. It catalyzes the exchange of GDP for GTP, activating RhoA, a member of the Rho family of small GTPases. RhoA plays a role in various cellular processes, including actin cytoskeleton organization, cell migration, and cell contraction. ARHGEF1, by activating RhoA, has implications in signal transduction pathways related to cell morphology, motility, and cell adhesion.

HAVCR1, also known as KIM-1 (Kidney Injury Molecule-1), has a size of approximately 38 kDa. This transmembrane protein is upregulated in the kidney after injury, making it a useful marker for renal damage. Besides its role as a hepatitis A virus receptor, HAVCR1 is also implicated in immune cell regulation, especially in T-cell responses.

FLT3 is a receptor tyrosine kinase with a size of approximately 158 kDa. It is expressed in early hematopoietic progenitor cells and plays a pivotal role in hematopoiesis, the process of blood cell formation. FLT3 regulates the proliferation and differentiation of hematopoietic stem cells and progenitor cells. Mutations in the FLT3 gene, particularly internal tandem duplications (ITDs), are commonly found in acute myeloid leukemia (AML) and are associated with a poor prognosis.

MAN1A2 is an enzyme that belongs to the mannosidase family, with a size of approximately 100 kDa. It is involved in the processing of mannose-rich oligosaccharides during the maturation of N-linked glycoproteins within the Golgi apparatus. Alterations or defects in this enzyme can affect glycoprotein processing and, consequently, cellular functions that rely on glycoproteins.

ACY3 is an enzyme with a size of approximately 42 kDa. It is responsible for hydrolyzing N-acetylated amino acids, converting them into amino acids and acetate, primarily within the cytoplasm of kidney cells. This process plays a role in amino acid metabolism and detoxification.

ADGRG1, also known as GPR56, is a member of the adhesion G protein-coupled receptor (GPCR) family. With a size of approximately 84 kDa, it plays roles in various biological processes, including neural migration during brain development and modulation of the immune response. Mutations in ADGRG1 have been linked to brain developmental disorders.

MYCBP2, often called PHR1 (Phr1 ubiquitin ligase), has a size of approximately 487 kDa. This protein functions as an E3 ubiquitin-protein ligase, promoting the attachment of ubiquitin to target proteins. MYCBP2 plays roles in axon guidance and synaptic development in neurons. Its malfunction or dysregulation can impact neuronal pathways and connections.

ITGB1 is a cell surface receptor protein with a size of approximately 88 kDa. It partners with various integrin alpha subunits to form heterodimeric integrin receptors that mediate cell-cell and cell-extracellular matrix interactions. These interactions play crucial roles in cellular processes like adhesion, migration, differentiation, and signal transduction. Dysregulation or mutations in ITGB1 can contribute to various pathologies, including impaired wound healing, tumor invasion, and metastasis.

CLEC4A, also known as DCIR (Dendritic Cell Immunoreceptor), is a member of the C-type lectin domain family. It has a size of approximately 26 kDa. CLEC4A is primarily expressed in dendritic cells and functions as an inhibitory receptor, modulating immune responses. It is believed to play roles in autoimmunity and inflammatory diseases.

MEP1B is an enzyme that belongs to the astacin family of metalloendopeptidases with a size of approximately 61 kDa. Found predominantly in the intestines, MEP1B plays a role in the digestion of dietary proteins and processing of polypeptides. Additionally, it may also be involved in the activation of proinflammatory cytokines and degradation of extracellular matrix proteins.

F13B is a component of coagulation factor XIII, with a size of approximately 80 kDa. Factor XIII is a transglutaminase that stabilizes the formation of the fibrin clot by crosslinking fibrin chains during the clotting process. F13B acts as a carrier and protective molecule for the active A subunits until activation.

FCN1, also known as M-ficolin, is approximately 35 kDa. It is one of the recognition molecules in the lectin pathway of the complement system, binding carbohydrate molecules on the surface of pathogens and playing a role in innate immunity by activating the complement cascade to eliminate invaders.

ADCYAP1R1 is a G-protein coupled receptor with a protein size of approximately 50 kDa. This receptor binds to pituitary adenylate cyclase-activating polypeptide (PACAP) and plays roles in diverse biological processes, including neurotransmission, vasodilation, and regulation of secretion. Dysregulation or mutations in this receptor have been implicated in various conditions, including migraine and post-traumatic stress disorder.

LILRA5, with a size of approximately 58 kDa, is part of the leukocyte immunoglobulin-like receptor (LIR) family. These receptors are involved in the modulation of immune responses. The exact function of LILRA5 is still under investigation, but like other LIRs, it likely plays a role in immune cell activation or inhibition.

HEPH is a multicopper oxidase with a size of approximately 130 kDa. Predominantly expressed in the intestines, it plays a pivotal role in iron homeostasis. HEPH facilitates the export of dietary iron from intestinal cells into the bloodstream by oxidizing ferrous iron, enabling its binding to transferrin.

CLEC10A, also known as MGL (Macrophage Galactose-Type Lectin), is of approximately 40 kDa. It is predominantly expressed on dendritic cells and recognizes specific carbohydrate structures. CLEC10A plays roles in pathogen recognition, cell-cell interactions, and potentially in modulating immune responses.

RABEPK is a protein associated with vesicle trafficking, with a size of approximately 72 kDa. It interacts with RAB9, a small GTPase involved in the transport of proteins between the endosomes and the trans-Golgi network. By binding to RAB9, RABEPK may play roles in vesicle tethering and fusion processes.

FCER2, commonly referred to as CD23, has a size of approximately 45 kDa. It is primarily expressed on B cells and acts as a low-affinity receptor for the Fc portion of IgE. This receptor is involved in the regulation of IgE synthesis and B-cell differentiation and growth. Alterations in its expression have implications in allergies and certain types of leukemia.

TG is a large glycoprotein with a size of approximately 330 kDa. It is synthesized in the thyroid gland and plays a fundamental role in thyroid hormone synthesis. TG acts as a precursor for the thyroid hormones thyroxine (T4) and triiodothyronine (T3), which regulate various metabolic processes in the body. Detection of autoantibodies against TG is commonly used as a diagnostic marker for autoimmune thyroid diseases, such as Hashimoto's thyroiditis.

CA3 is an enzyme of approximately 29 kDa that belongs to the carbonic anhydrase family. These enzymes catalyze the rapid conversion of carbon dioxide and water to bicarbonate and protons, playing a role in pH regulation and carbon dioxide transport. CA3 is predominantly found in skeletal muscles.

CCL8, also known as MCP-2, is a small cytokine with a size of approximately 13 kDa. It belongs to the CC chemokine family, attracting monocytes and lymphocytes. CCL8 is involved in inflammatory responses and plays a role in the pathogenesis of diseases, including atherosclerosis and rheumatoid arthritis.

CD22, a protein of approximately 140 kDa, is a member of the SIGLEC family of lectins. It is primarily expressed on B cells and acts as a modulator of B-cell activation. CD22 serves as a negative regulator, ensuring that B cells don't over-respond to antigens.

IL17A is a cytokine of approximately 20 kDa. It's produced mainly by activated T cells and plays a critical role in inflammation, particularly in autoimmune diseases. IL17A stimulates the production of other cytokines and chemokines, amplifying the inflammatory response.

IL7 is a cytokine with a size of approximately 25 kDa. It plays a crucial role in the development and maturation of T and B cells. IL7 is vital for lymphocyte survival, proliferation, and homeostasis.

KLHL41, a protein of approximately 70 kDa, is part of the kelch-like protein family. It's involved in muscle development and functions as an actin-organizing protein, playing a role in myofibril assembly.

KLRC1, often referred to as NKG2A, has a size of approximately 44 kDa. This receptor is expressed on natural killer (NK) cells and some T cells. It interacts with specific HLA class I molecules, regulating the cytotoxic activity of these immune cells.

FCRL1, with a protein size of approximately 60 kDa, belongs to the Fc receptor-like family. Expressed on B cells, its function is still not fully understood, but it may play roles in B-cell signaling or modulation.

IL17C is a cytokine of approximately 22 kDa. Part of the IL17 family, it's involved in inducing and mediating proinflammatory responses. IL17C plays a role in host defense against pathogens but can also be implicated in autoimmune and inflammatory diseases.

KLKB1, with a size of approximately 66 kDa, encodes plasma prekallikrein. Once activated, it plays roles in blood coagulation, blood pressure regulation, and inflammatory pathways.

IFNGR2, a protein of approximately 38 kDa, is a part of the interferon-gamma receptor complex. It's vital for immune responses against viral infections and intracellular bacteria, as well as for tumor control.

CST7, with a protein size of approximately 15 kDa, encodes cystatin F, an inhibitor of lysosomal proteinases. It's involved in immune system processes, especially within natural killer cells and cytotoxic T lymphocytes.

FLT3LG, or FLT3 ligand, is a protein of approximately 30 kDa. It stimulates the proliferation of hematopoietic stem cells and can induce the generation of dendritic cells.

CCL19, a chemokine of approximately 13 kDa, attracts dendritic cells, T cells, and B cells, guiding them within lymphoid tissues. It plays roles in immune surveillance and adaptive immune responses.

SERPINA3, with a protein size of approximately 55 kDa, is an acute phase protein. It inhibits cathepsin G and related proteases, playing a role in inflammation control and tissue remodeling.

KIRREL1, a protein of approximately 80 kDa, is a member of the immunoglobulin superfamily. It's involved in kidney and neural development, specifically in the formation of specialized cell-cell junctions.

LTA, also known as TNF-beta, is a cytokine of approximately 25 kDa. It's involved in immune responses, playing roles in lymphoid organ development and controlling immune cell functions.

AMPD3, an enzyme of approximately 90 kDa, catalyzes the conversion of adenosine monophosphate (AMP) to inosine monophosphate (IMP), playing roles in purine metabolism.

CCL2, also known as MCP-1, is a chemokine of approximately 13 kDa. It recruits monocytes, memory T cells, and dendritic cells to sites of tissue injury, infection, and inflammation.

DPEP2, an enzyme of approximately 50 kDa, participates in the metabolism of dipeptides, breaking them down into their amino acid components.

CFHR5, with a size of approximately 65 kDa, is part of the factor H protein family, which modulates the alternative pathway of the complement system. Mutations in CFHR5 are linked to kidney diseases.

F10, with a size of approximately 59 kDa, plays a critical role in the coagulation cascade, leading to blood clotting. It's activated by either the intrinsic or the extrinsic pathway and activates thrombin in the common pathway.

SERPIND1, a protein of approximately 50 kDa, is also known as heparin cofactor II. It inhibits thrombin and plays a role in anticoagulation.

CSF3, also known as G-CSF, is a cytokine of approximately 20 kDa. It stimulates the production of neutrophils in the bone marrow and their release into the bloodstream.

CCL13, also known as MCP-4, is a chemokine of approximately 11 kDa. It recruits eosinophils, monocytes, and lymphocytes, playing roles in allergic reactions and other inflammatory responses.

PFKFB2, an enzyme of approximately 55 kDa, regulates glycolysis by producing fructose-2,6-bisphosphate, a potent activator of phosphofructokinase-1.

CSF1, also known as M-CSF, is a cytokine of approximately 45 kDa. It controls the production, differentiation, and function of macrophages.

APOF, a protein of approximately 34 kDa, is a component of the lipoprotein particles, playing a role in lipid transport and metabolism. Its specific function in these processes is still under investigation.

LMOD1 is a protein with a size of approximately 60 kDa. Found mainly in smooth muscles, LMOD1 plays a pivotal role in the nucleation of actin filaments, facilitating muscle contraction. Mutations in LMOD1 can be linked to certain myopathies.

RNASE10 has a size of approximately 16 kDa. Part of the ribonuclease A superfamily, its function isn't as well-characterized as other family members, but like its counterparts, it is likely involved in the cleavage of RNA.

APCS is a glycoprotein of approximately 25 kDa. This protein is a member of the pentraxin family and binds to various ligands, such as phosphocholine and apoptotic cells. It's often associated with amyloid deposits, being a constituent of all types of amyloid plaques.

CEP20 is approximately 20 kDa in size. This protein is involved in centrosome-related processes and is vital for the correct assembly and function of the centrosome, which is crucial for cell division.

NAMPT is an enzyme of approximately 52 kDa. It plays a critical role in the biosynthesis of nicotinamide adenine dinucleotide (NAD), acting in the salvage pathway to produce NAD from nicotinamide. This enzyme has been implicated in various physiological and pathophysiological processes, including metabolism, aging, and inflammation.

OLR1, often referred to as LOX-1, has a size of approximately 50 kDa. This receptor recognizes and binds to oxidized LDL, playing a crucial role in atherosclerosis. Its increased expression can be found in atherosclerotic lesions.

ADAMTSL2 is a protein of approximately 120 kDa. Though it shares similarities with the ADAMTS family of metalloproteases, it lacks the protease domain. Mutations in ADAMTSL2 are associated with geleophysic dysplasia, a rare genetic disorder.

VEGFA, with a size ranging from 21-45 kDa based on its isoforms, is a potent mediator of angiogenesis, the process by which new blood vessels form. It stimulates endothelial cell growth and migration. VEGFA has been targeted in cancer therapies due to its role in promoting tumor angiogenesis.

IL15 is a cytokine of approximately 14-15 kDa. This protein plays vital roles in the stimulation and proliferation of natural killer (NK) cells and CD8 T cells. It's involved in immune responses against viral infections and contributes to immune-surveillance against tumors.

EGF is a growth factor with a size of approximately 6 kDa. It stimulates the growth of various epidermal and epithelial tissues in vivo and in vitro and has roles in wound healing. The EGF receptor (EGFR) pathway is targeted in several cancer therapies.

CFH, a protein of approximately 155 kDa, is a regulator of the complement system's alternative pathway. It binds to host cell surfaces and protects them from complement attack. Mutations in CFH can lead to conditions like atypical hemolytic uremic syndrome and age-related macular degeneration.

TNFSF10, also known as TRAIL, has a size of approximately 32 kDa. It induces apoptosis in tumor cells, making it a focus for cancer therapy. However, not all cancer cells are sensitive to TRAIL-mediated apoptosis.

TNF is a cytokine of approximately 17 kDa, playing a key role in inflammation, immune system development, apoptosis, and lipid metabolism. Dysregulation of TNF production has been implicated in various diseases, including autoimmune diseases, insulin resistance, and cancer.

MIP 3-α, also known as CCL20, is a chemokine of approximately 10 kDa. It attracts lymphocytes and neutrophils to sites of inflammation and has been implicated in the pathogenesis of various inflammatory diseases, including psoriasis and rheumatoid arthritis.

Exemplary biomarkers and corresponding UniProt identifiers are shown in Table 1.

TABLE 1
Exemplary Biomarkers.

| Biomarker Abbreviation | UniProt Identifier |
|---|---|
| CD1C | P29017 |
| DLG4 | P78352 |
| TXNDC15 | Q96J42 |
| SOD2 | P04179 |
| TREML1 | Q86YW5 |
| IGDCC4 | Q8TDY8 |
| LMNB2 | Q03252 |
| GNAS | Q5JWF2 |
| CLMP | Q9H6B4 |
| GFAP | P14136 |
| NEFL | P07196 |
| CXCL-13 | O43927 |
| APLP1 | P51693 |
| MOG | Q16653 |
| OPG | O00300 |
| VCAN | P13611 |
| CDCP1 | Q9H5V8 |
| TNSF13b | Q9Y275 |
| CNTN2 | Q02246 |
| CXCL9 | Q07325 |
| SERPINA9 | Q86WD7 |
| OPN | P10451 |
| CD6 | P30203 |
| TNFRSF10a | O00220 |
| CCL20 | P78556 |
| COL4A1 | P02462 |
| GH | P01241 |
| IL-12 | P29460 |
| PRTG | Q2VWP7 |
| CXCL10 | P02778 |
| IL15 | P40933 |
| EGF | P01133 |
| CXCL11 | O14625 |
| CFH | P08603 |
| TNFSF10 | P50591 |
| IL18 | Q14116 |
| IL6 | P05231 |
| TNF | P01375 |
| HAVCR1 | Q96D42 |
| FLT3 | P36888 |
| MAN1A2 | O60476 |
| ACY3 | Q96HD9 |
| ARHGEF1 | Q92888 |
| ADGRG1 | Q9Y653 |
| MYCBP2 | O75592 |
| ITGB1 | P05556 |
| CLEC4A | Q9UMR7 |
| MEP1B | Q16820 |
| F13B | P05160 |
| FCN1 | O00602 |
| ADCYAP1R1 | P41586 |
| LILRA5 | A6NI73 |
| HEPH | Q9BQS7 |
| CLEC10A | Q8IUN9 |
| RABEPK | Q7Z6M1 |
| FCER2 | P06734 |
| TG | P01266 |
| CXCL12 | P48061 |
| CA3 | P07451 |
| CXCL8 | P10145 |
| CCL8 | P80075 |
| CD22 | P20273 |
| IL17A | Q16552 |
| IL7 | P13232 |
| KLHL41 | O60662 |
| KLRC1 | P26715 |
| FCRL1 | Q96LA6 |
| IL17C | Q9POM4 |
| KLKB1 | P03952 |
| IFNGR2 | P38484 |
| CST7 | O76096 |
| FLT3LG | P49771 |
| CCL19 | Q99731 |
| GFRA2 | O00451 |
| SERPINA3 | P01011 |
| KIRREL1 | Q96J84 |
| LTA | P01374 |
| AMPD3 | Q01432 |
| CCL2 | P13500 |
| DPEP2 | Q9H4A9 |
| CFHR5 | Q9BXR6 |
| F10 | P00742 |
| SERPIND1 | P05546 |
| CSF3 | P09919 |
| CCL13 | Q99616 |
| PFKFB2 | O60825 |
| CSF1 | P09603 |
| APOF | Q13790 |
| MMP12 | P39900 |
| LMOD1 | P29536 |
| RNASE10 | Q5GAN6 |
| APCS | P25054 |
| MMP1 | P03956 |
| CEP20 | Q96NB1 |
| NAMPT | P43490 |
| OLR1 | P78380 |
| ADAMTSL2 | Q86TH1 |
| VEGFA | P15692 |

### VI. Assays

As shown in FIG. 1A, the system environment 100 involves implementing a marker quantification assay 120 for evaluating expression levels of one or more biomarkers. Examples of an assay (e.g., marker quantification assay 120) for one or more markers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system.

Various immunoassays designed to quantitate markers can be used in screening including multiplex assays. Measuring the concentration of a target marker in a sample or fraction thereof can be accomplished by a variety of specific assays. For example, a conventional sandwich type assay can be used in an array, ELISA, RIA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

Protein based analysis, using an antibody that specifically binds to a polypeptide (e.g. marker), can be used to quantify the marker level in a test sample obtained from a subject. In various embodiments, an antibody that binds to a marker can be a monoclonal antibody. In various embodiments, an antibody that binds to a marker can be a polyclonal antibody. For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five, six, seven, eight, nine, ten, eleven, twelve, thirteen, fourteen, fifteen, sixteen, seventeen, eighteen, nineteen, twenty, twenty one, or more markers.

In various embodiments, the multiplex assay involves the use of oligonucleotide labeled antibody probes that bind to target biomarkers and allow for subsequent quantification of biomarkers. One example of a multiplex assay that involves oligonucleotide labeled antibody probes is the Proximity Extension Assay (PEA) technology (Olink Proteomics). Briefly, a pair of oligonucleotide labeled antibodies bind to a biomarker, wherein the two oligonucleotide sequences are complementary to one another. Thus, only when both antibodies bind to the target biomarker will the oligonucleotide sequences hybridize with one another. Mismatched oligonucleotide sequences (which occurs due to non-specific binding of antibodies or cross-reactivity of antibodies) will not hybridize and therefore, will not result in a readout. Hybridized oligonucleotide sequences undergo nucleic acid extension and amplification, followed by quantification using microfluidic qPCR. The quantified levels correlate to the quantitative expression values of the respective biomarkers.

In various embodiments, the multiplex assay involves the use of bead conjugated antibodies (e.g., capture antibodies) that enable the binding and detection of biomarkers. One example of a multiplex assay involving bead conjugated antibodies is Luminex's xMAP® Technology. Here, bead conjugated antibodies are added to the sample along with biotinylated detection antibodies. Both antibodies are specific to the biomarkers of interest and therefore, form an antibody-antigen sandwich. Streptavidin is further added, which binds to the biotinylated detection antibodies and enables detection of the complex. The Luminex 200™ or FlexMap® analyzer are employed to identify and quantify the amount of the biomarker in the sample. In various embodiments, the multiplex assay represents an improvement over Luminex's xMAP® technology, such as the Multi-Analyte Profile (MAP) technology by Myriad Rules Based Medicine (RBM), Inc.

In various embodiments, prior to implementation of a marker quantification assay 120 (e.g., an immunoassay), a sample obtained from a subject can be processed. In various embodiments, processing the sample enables the implementation of the marker quantification assay 120 to more accurately evaluate expression levels of one or more biomarkers in the sample.

In various embodiments, the sample from a subject can be processed to extract biomarkers from the sample. In one embodiment, the sample can undergo phase separation to separate the biomarkers from other portions of the sample. For example, the sample can undergo centrifugation (e.g., pelleting or density gradient centrifugation) to separate larger and/or more dense entities in the sample (e.g., cells and other macromolecules) from the biomarkers. Other examples include filtration (e.g., ultrafiltration) to phase separate the biomarkers from other portions of the sample.

In various embodiments, the sample from a subject can be processed to produce a sub-sample with a fraction of biomarkers that were in the sample. In various embodiments, producing a fraction of biomarkers can involve performing a protein fractionation procedure. One example of protein fractionation procedures include chromatography (e.g., gel filtration, ion exchange, hydrophobic chromatography, or affinity chromatography). In particular embodiments, the protein fractionation procedure involves affinity purification or immunoprecipitation where biomarkers are bound by specific antibodies. Such antibodies can be immobilized on a support, such as a magnetic particle or nanoparticle or a plate.

In various embodiments, the sample from the subject is processed to extract biomarkers from the sample and further processed to produce a sub-sample with a fraction of extracted biomarkers. Altogether, this enables a purified sub-sample of biomarkers that are of particular interest. Thus, implementing an assay (e.g., an immunoassay) for evaluating expression levels of the biomarkers of particular interest can be more accurate and of higher quality. In various embodiments, the biomarkers of particular can be biomarkers of a biomarker panel, embodiments of which are described herein. As an example, biomarkers of a biomarker panel can include two or more of: CD1C, DLG4, TXNDC15, SOD2, TREML1, IGDCC4, LMNB2, GNAS, CLMP, GFRA2, ARHGEF1, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, IL15, GFAP, APLP1, MOG, OPG, VCAN, CDCP1, TNFSF13b, CNTN2, CXCL9, SERPINA9, OPN, CD6, TNFRSF10a, CCL20, COL4A1, GH, IL-12, PRTG, CXCL10, EGF, CXCL11, CFH, TNFSF10, IL18, IL6, TNF, CXCL13, NEFL, CCL20/MIP 3-α, and FLRT2.

### VII. Therapeutic Agents and Compositions for Therapeutic Agents

In various embodiments, a therapeutic agent is provided to an individual prior to and/or subsequent to obtaining the sample from the individual and determining quantitative expression values of one or more markers in the obtained sample. As one example, a predictive model that receives the quantitative expression values predicts that an individual is to be diagnosed with multiple sclerosis and a therapeutic agent is to be provided. In another example, the predictive model predicts that a provided therapeutic agent is demonstrating therapeutic efficacy against a multiple in a previously diagnosed individual.

In various embodiments the therapeutic agent is a biologic, e.g. a cytokine, antibody, soluble cytokine receptor, anti-sense oligonucleotide, siRNA, etc. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists, e.g. IL-1Ra, IL-1 Trap, sIL-4Ra, etc. Also included are biosimilar or bioequivalent drugs to the active agents set forth herein.

Therapeutic agents for multiple sclerosis include corticosteroids, plasma exchange, ocrelizumab (Ocrevus®), IFN-β(Avonex®, Betaseron®, Rebif®, Extavia®, Plegridy®), Glatiramer acetate (Copaxone®, Glatopa®), anti-VLA4 (Tysabri, natalizumab), dimethyl fumarate (Tecfidera®, Vumerity®), teriflunomide (Aubagio®), monomethyl fumarate (Bafiertam™), ozanimod (Zeposia®), siponimod (Mayzent®), fingolimod (Gilenya®), anti-CD52 antibody (e.g., alemtuzumab (Lemtrada®), mitoxantrone (Novantrone®), methotrexate, cladribine (Mavenclad®, simvastatin, and cyclophosphamide. In addition or alternative to therapeutic agents, other treatments for multiple sclerosis include lifestyle changes such as physical therapy or a change in diet. The method also provide for combination therapy of one or more therapeutic agents and/or additional treatments, where the combination can provide for additive or synergistic benefits.

A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients. Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

Such a pharmaceutical composition may be administered for prophylactic (e.g., before diagnosis of a patient with multiple sclerosis) or for treatment (e.g., after diagnosis of a patient with multiple sclerosis) purposes. Preventing, prophylaxis or prevention of a disease or disorder as used in the context of this invention refers to the administration of a composition to prevent the occurrence or onset of multiple sclerosis or some or all of the symptoms of multiple sclerosis or to lessen the likelihood of the onset of a disease or disorder. Treating, treatment, or therapy of multiple sclerosis shall mean slowing, stopping or reversing the disease's progression by administration of treatment according to the present invention. In the preferred embodiment, treating multiple sclerosis means reversing the disease's progression, ideally to the point of eliminating the disease itself.

### VIII. Disease Activity in a Subject

Methods described herein focus on assessing disease activity in a subject by applying quantitative expression levels of biomarkers as input to a predictive model. In various embodiments, the subject is classified in a category based on the predicted assessment of the disease activity. To classify the subject, the prediction for the subject may be compared to results of individuals that have been previously classified in a clinically diagnosed category. For example, individuals may be clinically categorized in one of a diagnosis of MS (e.g., presence of MS), a categorization of a subtype of MS (e.g., radiologically isolated syndrome (RIS), clinically isolated syndrome (CIS), relapsing-remitting MS (RRMS), primary progressive MS (PPMS), and secondary-progressive MS (SPMS)), a categorization in a quiescent or exacerbated state, a categorization in a level of disability according to the expanded disability status scale (EDSS), an identified clinical response to a therapy, and a clinical identification of a risk of developing MS. Clinical categories can also be determined using any of a MS functional composite (MSFC), timed 25-foot walk (T25Fw), 9-hole peg test (9HPT), or patient-reported outcomes (e.g., patient determined disease steps (PDDS)/MSSS (patient-derived disability status scale), PRO measurement information system (PROMIS), or Multiple Sclerosis Rating Scale, Revised (MSRS-R)). Individuals may be clinically categorized based on a measurable for MS disease activity, such as a particular number of gadolinium enhancing lesions (e.g., subtle disease activity) or the presence of at least one gadolinium enhancing lesion (e.g., general disease activity). Clinical categorization can also occur based on other radiographic measures including T2 lesions (new or enlarging), slowly expanding lesions, rim-expanding lesions, Brain Parenchymal Fraction (BPF) & percentage change, Gray matter fraction, White matter fraction, Thalamic volume, Cortical gray matter volume, Deep gray matter volume, or Radiologist notes of auxiliary features (e.g. Dawson's Fingers). Categorization of previously individuals may occur based on clinical standards.

Clinical diagnosis of MS can occur through various methods. As an example, a clinical diagnosis of MS can be made through magnetic resonance imaging (MRI) of the brain and spinal cord to identify lesions or plaques that form as a result of MS. The McDonald criteria can be employed in making the diagnosis. Clinical diagnosis of MS can also occur through a lumbar puncture (spinal tap) that observes abnormalities in antibody concentrations in the spinal fluid due to the presence of MS. Clinical diagnosis of MS can also occur through evoked potential tests, where electrical signals produced by neurons of the nervous system are recorded in response to a stimulus. An impaired transmission is indicative of the presence of MS.

Clinical categorization of a patient previously diagnosed with MS in a quiescent state versus an exacerbated state can depend on a variety of factors. Namely, a patient can be clinically categorized in an exacerbated state after presenting with a new disease that is related to MS (e.g., a comorbidity or symptom such as clinical depression or optic neuritis). As another example, a patient is clinically categorized in an exacerbated state if the patient presents with significant worsening of symptoms. Examples may include a worsening of balance and/or mobility, vision, pain in the eye, fatigue, and/or heart-related problems. Patients previously diagnosed with MS can be clinically categorized in a quiescent state if the patient does not present with a new disease or a change or worsening of symptoms.

Determination that a patient previously diagnosed with MS is responding to a therapy can be dependent on a variety of clinical variables. For example, a response to therapy can be determined based on the occurrence or lack of a relapse. A patient can be deemed responsive to a therapy if relapses do not occur. A response to therapy can also be determined based on a total number of relapses, a time to a first relapse, the patient's EDSS score, a change in the patient's EDSS score (e.g., an increase in the score corresponds to a lack of response to therapy), a change in MRI status (e.g., the development of additional lesions or plaques corresponds to a lack of response to therapy).

Patients can be clinically categorized in a level of disability, which can be a measure of disease progression. For example, the EDSS can be used to determine a severity of MS in a patient. Therefore, patients are categorized in categories that correspond to an EDSS score between 1.0 and 10.0 in 0.5 point intervals. Generally, EDSS scores of 1.0 to 4.5 refer to patients with MS who are able to walk without any aid. EDSS scores of 5.0 to 9.5 refer to patients with MS whose ability to walk is impaired, with a higher score corresponding to a higher degree of impairment. In various embodiments, an EDSS score less than 6 indicates mild/moderate MS disease progression. In various embodiments an EDSS score greater than or equal to 6 indicates severe MS disease progression. In particular embodiments, an EDSS score between 0-3.0 represents mild MS, an EDSS score between 3.5-5.5 represents moderate MS, an EDSS score between 6.0-9.5 represents severe MS.

As another example, patients can be clinically categorized in a level of disability according to PDDS, which is a validated scale as a self-reported proxy for EDSS and therefore, can be used to determine a severity of MS in a patient. A PDDS score of 0 indicates a normal disability level with mild, sensory symptoms with no limit on activity. A PDDS score of 1 indicates a mild disability with minor, noticeable symptoms that have only a small effect on lifestyle. A PDDS score of 2 indicates moderate disability with no limitation in walking ability but significant problems that limit daily activities in other ways. A PDDS score of 3 indicates gait disability with interferences with activities such as walking. A PDDS score of 4 indicates early cane disability which is characterized by use of a cane or single crutch for walking all or part of the time (e.g., can walk 25 feet in 20 seconds without a cane or crutch). A PDDS score of 5 indicates late cane disability which is character the use of a cane or crutch to walk 25 feet. A PDDS score of 6 indicates bilateral support disability which is characterize by the need to use 2 canes, crutches, or a walker to walk 25 feet. A PDDS score of 7 indicates wheelchair/scooter disability in which the individual's main form of mobility is a wheelchair/scooter. A PDDS score of 8 indicates bedridden disability in which the individual is unable to sit in a wheelchair for more than 1 hour. In various embodiments, a PDDS score less than or equal to 4 indicates disease progression to mild/moderate MS disability. In various embodiments a PDDS score greater than 4 indicates disease progression to severe MS disability. In particular embodiments, a PDDS score between 0 and 1 represents mild MS, a PDDS score between 2-4 represents moderate MS, and a PDDS score between 5-8 represents severe MS.

As another example, patients can be clinically categorized in a level of disability according to PROMIS measure. Generally, PROMIS scores are based on the T-score metric in which a score of 50 represents a mean score of a corresponding reference population with a standard deviation of 10. Therefore, a score of 40 for an individual indicates that the individual is one standard deviation lower than the mean of the corresponding reference population (e.g., score of 40 indicates that individual's MS disability is a standard deviation lower than the MS disability of the mean of the population). A score of 60 for an individual indicates that the individual is one standard deviation higher than the mean of the corresponding reference population (e.g., score of 60 indicates that individual's MS disability is a standard deviation higher than the MS disability of the mean of the population).

As another example, patients can be clinically categorized in a level of disability according to a MSRS-R measure. Generally, MSRS-R scores can be measured according to the following items: 1) Walking, 2) Using your arms and hands, 3) Vision (with glasses or contacts if you use them), 4) Speaking clearly, 5) Swallowing, 6) Thinking, Memory, or Cognition, 7) Numbness, Tingling, Burning Sensation or Pain, and 8) Bowel or bladder. Each of the items is rated according to a level of disability: “0—Normal status”, “1—Symptoms causing no disability”, “2—Mild disability not requiring help from others”, “3—Moderate disability requiring help from others”, and 4—“Total loss of function, maximal help required”). Total MSRS-R score represents the sum of the scores across the 8 items.

PIRA may be defined as an event of experiencing confirmed disability accumulation (CDA) generally measured using the EDSS scale at 6 months (or alternatively a longer duration) during a period free of relapses (PFRs). CDA may also be determined using or incorporating alternate progression metrics including radiographic endpoints. A PFR is the time between 2 consecutive relapses, starting 3 months after a relapse (or 6 months after the first demyelinating event). The first EDSS score may be obtained at least 6 months after the first attack or 3 months after any other attack was referred to as the baseline EDSS score and rebaseline EDSS score, respectively. The date of PIRA may be the date of the confirmation of the CDA. Any other episodes of CDA that do not qualify for PIRA (i.e., which occurred outside the PFR) may be considered to be RAW events. Those patients with at least 1 CDA but who do not present with any PIRA event may be considered patients with RAW. For clarity, patients with RAW may have an acute focal inflammatory event that manifested as a clinical relapse and/or radiographic evidence of disease activity (gadolinium enhanced lesion(s) or new/enlarging T2 lesion(s)) that resulted in the observed CDA.

All patients with PIRA may be classified into early PIRA or late PIRA groups, depending on whether the first PIRA event occurred within the first 5 years since their first attack or afterward, respectively; the choice of a 5-year cutoff is taken from previous longitudinal studies of primary progressive MS, which considered early disease to be disease with a duration less than 5 years. Patients with PIRA may further be classified into active PIRA or nonactive PIRA groups, depending on the presence or absence, respectively, of new T2 lesions observed in the 2 years before developing PIRA. The latter classification may be applied on a subcohort of patients with a brain MRI scan available within the 2 years before developing PIRA.

In some embodiments, the biomarker panel provided herein may be used to assess RAW. In some embodiments, the biomarker panel comprising at least one biomarker selected from GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2 may be used to assess RAW.

In some embodiments, the biomarker panel provided herein may be used to assess PIRA. In some embodiments, the biomarker panel comprising at least one biomarker selected from CD1C, DLG4, TXNDC15, SOD2, TREML1, IGDCC4, LMNB2, GNAS, CLMP, GFRA2, ARHGEF1, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL may be used to assess PIRA.

Accordingly, in various non-limiting embodiments, RAW may be used as a classification of acute disease activity. In various embodiments, the prediction of multiple sclerosis disease progression is a prediction of relapse associated worsening (RAW).

In other non-limiting embodiments, PIRA may be used as a classification of chronic deterioration of neurologic functions. In various embodiments, the prediction of multiple sclerosis disease progression is a prediction of progression independent of relapse activity (PIRA).

In various embodiments, the prediction of multiple sclerosis disease progression is a prediction of relapse associated worsening (RAW) and progression independent of relapse activity (PIRA).

### IX. Computer Implementation

The methods of the invention, including the methods of assessing multiple sclerosis activity (e.g., multiple sclerosis disease progression) in an individual, are, in some embodiments, performed on one or more computers.

For example, the building and deployment of a predictive model and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of a predictive model of this invention. Such data can be used for a variety of purposes, such as patient monitoring, treatment considerations, and the like. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods of assessing multiple sclerosis disease activity (e.g., multiple sclerosis disease progression) in an individual, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### VIII.A. Example Computer

FIG. 2 illustrates an example computer 200 for implementing the entities shown in FIGS. 1A-1C. The computer 200 includes at least one processor 202 coupled to a chipset 204. The chipset 204 includes a memory controller hub 220 and an input/output (I/O) controller hub 222. A memory 206 and a graphics adapter 212 are coupled to the memory controller hub 220, and a display 218 is coupled to the graphics adapter 212. A storage device 208, an input device 214, and network adapter 216 are coupled to the I/O controller hub 222. Other embodiments of the computer 200 have different architectures.

The storage device 208 is a non-transitory computer-readable storage medium such as a hard drive, compact disk read-only memory (CD-ROM), DVD, or a solid-state memory device. The memory 206 holds instructions and data used by the processor 202. The input interface 214 is a touch-screen interface, a mouse, track ball, or other type of pointing device, a keyboard, or some combination thereof, and is used to input data into the computer 200. In some embodiments, the computer 300 may be configured to receive input (e.g., commands) from the input interface 214 via gestures from the user. The graphics adapter 212 displays images and other information on the display 218. The network adapter 216 couples the computer 200 to one or more computer networks.

The computer 200 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 208, loaded into the memory 206, and executed by the processor 202.

The types of computers 200 used by the entities of FIG. 1 can vary depending upon the embodiment and the processing power required by the entity. For example, the disease progression system 130 can run in a single computer 200 or multiple computers 200 communicating with each other through a network such as in a server farm. The computers 200 can lack some of the components described above, such as graphics adapters 212, and displays 218.

### X. Kit Implementation

Also disclosed herein are kits for assessing multiple sclerosis disease activity (e.g., multiple sclerosis disease progression) in an individual. Such kits can include reagents for detecting expression levels of one or biomarkers and instructions for assessing disease activity (e.g., multiple sclerosis disease progression) based on the detected expression levels.

The detection reagents can be provided as part of a kit. Thus, the invention further provides kits for detecting the presence of a panel of biomarkers of interest in a biological test sample. A kit can comprise a set of reagents for generating a dataset via at least one protein detection assay (e.g., immunoassay) that analyzes the test sample from the subject. In various embodiments, the set of reagents enable detection of quantitative expression levels of biomarkers from any one of Tables 6-8. In particular embodiments, the set of reagents enable detection of quantitative expression levels of biomarkers categorized as Tier 1, Tier 2, or Tier 3 biomarkers in Tables 6-8. In certain aspects, the reagents include one or more antibodies that bind to one or more of the markers. The antibodies may be monoclonal antibodies or polyclonal antibodies. In some aspects, the reagents can include reagents for performing ELISA including buffers and detection agents.

A kit can include instructions for use of a set of reagents. For example, a kit can include instructions for performing at least one biomarker detection assay such as an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

In various embodiments, the kits include instructions for practicing the methods disclosed herein (e.g., methods for training or deploying a predictive model to predict an assessment of disease activity, such as multiple sclerosis disease progression). These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### XI. Systems

Further disclosed herein are system for analyzing quantitative expression levels of biomarkers for assessing disease activity (e.g., multiple sclerosis disease progression). In various embodiments, such a system can include a set of reagents for detecting expression levels of biomarkers in the biomarker panel, an apparatus configured to receive a mixture of the set of reagents and a test sample obtained from a subject to measure the expression levels of the soluble mediators, and a computer system communicatively coupled to the apparatus to obtain the measured expression levels and to implement the predictive model to assess the disease activity (e.g., multiple sclerosis disease progression).

The set of reagents enable the detection of quantitative expression levels of the biomarkers in the biomarker panel. In various embodiments, the set of reagents involve reagents used to perform an assay, such as an assay or immunoassay as described above. For example, the reagents include one or more antibodies that bind to one or more of the biomarkers. The antibodies may be monoclonal antibodies or polyclonal antibodies. As another example, the reagents can include reagents for performing ELISA including buffers and detection agents.

The apparatus is configured to detect expression levels of biomarkers in a mixture of a reagent and test sample. For example, the apparatus can determine quantitative expression levels of biomarkers through an immunologic assay or assay for nucleic acid detection. The mixture of the reagent and test sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading to generate quantitative expression values of biomarkers. Examples of an apparatus include a plate reader (e.g., a luminescent plate reader, absorbance plate reader, fluorescence plate reader), a spectrometer, and a spectrophotometer.

The computer system, such as example computer 200 described in FIG. 2, communicates with the apparatus to receive the quantitative expression values of biomarkers. The computer system implements, in silico, a predictive model to analyze the quantitative expression values of the biomarkers to predict an assessment of the disease activity (e.g., multiple sclerosis disease progression).

### XII. Additional Embodiments

Additionally disclosed herein are methods for predicting multiple sclerosis disease progression in a subject, the method comprising: obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFAP, NEFL, CXCL-13, APLP1, MOG, OPG, VCAN, CDCP1, TNFSF13b, CNTN2, CXCL9, SERPINA9, OPN, CD6, TNFRSF10a, CCL20, FLRT2, COL4A1, GH, IL-12, PRTG, CXCL10, IL15, EGF, CXCL11, CFH, TNFSF10, IL18, IL6, TNF, HAVCR1, FLT3, MAN1A2, ACY3, ARHGEF1, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, GFRA2, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, and VEGFA; and generating a prediction of multiple sclerosis disease progression by applying a predictive model to the expression levels of the plurality of biomarkers.

Also disclosed herein are methods for predicting multiple sclerosis disease progression in a subject, the method comprising: obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, IL15, NEFL, GFAP, and VEGFA, and optionally comprises at least one or more of: CXCL-13, APLP1, MOG, OPG, VCAN, CDCP1, TNFSF13b, CNTN2, CXCL9, SERPINA9, OPN, CD6, TNFRSF10a, CCL20, COL4A1, GH, IL-12, PRTG, CXCL10, EGF, CXCL11, CFH, TNFSF10, IL18, IL6, TNF, CXCL13, NEFL, CCL20/MIP 3-α, FLRT2; and generating a prediction of multiple sclerosis disease progression by applying a predictive model to the expression levels of the plurality of biomarkers.

In various embodiments, the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL.

In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and GFAP.

In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and NEFL.

In various embodiments, a performance of the predictive model is characterized by an AUROC of at least 0.63, at least 0.66, at least 0.67, at least 0.69, at least 0.70, at least 0.74, at least 0.75, at least 0.78, or at least 0.81.

In various embodiments, the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

In various embodiments, a performance of the predictive model is characterized by an AUROC of at least 0.68, at least 0.69, at least 0.70, at least 0.71, at least 0.72, at least 0.73, at least 0.74, at least 0.76, at least 0.77, at least 0.81, at least 0.82, at least 0.83, or at least 0.86.

In various embodiments, the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL.

In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, and GFAP.

In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, and NEFL.

In various embodiments, a performance of the predictive model is characterized by an AUROC of at least 0.50, at least 0.51, at least 0.64, at least 0.70, at least 0.71, at least 0.72, at least 0.73, at least 0.74, at least 0.75, at least 0.76, at least 0.77, at least 0.79, at least 0.80, at least 0.81, at least 0.82, at least 0.83, at least 0.86, or at least 0.87.

In various embodiments, the prediction of multiple sclerosis disease progression is a prediction of relapse associated worsening (RAW).

In various embodiments, the prediction of multiple sclerosis disease progression is a prediction of progression independent of relapse activity (PIRA).

In various embodiments, the prediction of multiple sclerosis disease progression is a measure of expanded disability status scale (EDSS) score.

In various embodiments, an expanded disability status scale (EDSS) score less than 6 indicates a mild/moderate MS disease progression and a EDSS score greater than or equal to 6 indicates a severe MS disease progression.

In various embodiments, the prediction of multiple sclerosis disease progression is a measure of a patient determined disease steps (PDDS) score.

In various embodiments, the prediction of multiple sclerosis disease progression is a patient determined disease steps (PDDS) score differentiating between severe and mild/moderate MS disease progression.

In various embodiments, a patient determined disease steps (PDDS) score less than or equal to 4 indicates a mild/moderate MS disease progression and a PDDS score greater than 4 indicates a severe MS disease progression.

In various embodiments, the prediction of multiple sclerosis disease progression is a PRO measurement information system (PROMIS) score.

In various embodiments, the prediction of multiple sclerosis disease progression is a Multiple Sclerosis Rating Scale, Revised (MSRS-R) score.

In various embodiments, the prediction of multiple sclerosis disease progression is a MRI-based volumetric measurement.

In various embodiments, the MRI-based volumetric measurement is one of whole brain atrophy, brain parenchymal fraction (BPF), white matter atrophy, or gray matter atrophy.

In various embodiments, the multiple sclerosis is one of relapsing-remitting multiple sclerosis (RRMS), secondary progressive multiple sclerosis (SPMS), primary-progressive multiple sclerosis (PPMS), progressive relapsing multiple sclerosis (PRMS), or clinically isolated syndrome (CIS).

Further disclosed herein are methods for predicting multiple sclerosis disease progression in a subject, the method comprising: obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL; and generating a prediction of multiple sclerosis disease progression by applying a predictive model to the expression levels of the plurality of biomarkers.

In various embodiments, the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and GFAP.

In various embodiments, the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and NEFL.

In various embodiments, a performance of the predictive model is characterized by an AUROC of at least 0.63, at least 0.66, at least 0.67, at least 0.69, at least 0.70, at least 0.74, at least 0.75, at least 0.78, or at least 0.81.

In various embodiments, the prediction of multiple sclerosis disease progression is a prediction of relapse associated worsening (RAW).

In various embodiments, the prediction of multiple sclerosis disease progression is a prediction of progression independent of relapse activity (PIRA).

In various embodiments, the prediction of multiple sclerosis disease progression is a measure of expanded disability status scale (EDSS) score.

In various embodiments, an expanded disability status scale (EDSS) score less than 6 indicates a mild/moderate MS disease progression and a EDSS score greater than or equal to 6 indicates a severe MS disease progression.

In various embodiments, the prediction of multiple sclerosis disease progression is a measure of a patient determined disease steps (PDDS) score.

In various embodiments, the prediction of multiple sclerosis disease progression is a patient determined disease steps (PDDS) score differentiating between severe and mild/moderate MS disease progression.

In various embodiments, a patient determined disease steps (PDDS) score less than or equal to 4 indicates a mild/moderate MS disease progression and a PDDS score greater than 4 indicates a severe MS disease progression.

In various embodiments, the prediction of multiple sclerosis disease progression is a PRO measurement information system (PROMIS) score.

In various embodiments, the prediction of multiple sclerosis disease progression is a Multiple Sclerosis Rating Scale, Revised (MSRS-R) score.

In various embodiments, the prediction of multiple sclerosis disease progression is a MRI-based volumetric measurement.

In various embodiments, the MRI-based volumetric measurement is one of whole brain atrophy, brain parenchymal fraction (BPF), white matter atrophy, or gray matter atrophy.

In various embodiments, the multiple sclerosis is one of relapsing-remitting multiple sclerosis (RRMS), secondary progressive multiple sclerosis (SPMS), primary-progressive multiple sclerosis (PPMS), progressive relapsing multiple sclerosis (PRMS), or clinically isolated syndrome (CIS).

Disclosed herein are methods for predicting multiple sclerosis disease progression in a subject, the method comprising: obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2; and generating a prediction of multiple sclerosis disease progression by applying a predictive model to the expression levels of the plurality of biomarkers.

Additionally disclosed herein is a non-transitory computer readable medium comprising instructions that, when executed by a processor, cause the processor to: obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFAP, NEFL, CXCL-13, APLP1, MOG, OPG, VCAN, CDCP1, TNFSF13b, CNTN2, CXCL9, SERPINA9, OPN, CD6, TNFRSF10a, CCL20, FLRT2, COL4A1, GH, IL-12, PRTG, CXCL10, IL15, EGF, CXCL11, CFH, TNFSF10, IL18, IL6, TNF, HAVCR1, FLT3, MAN1A2, ACY3, ARHGEF1, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, GFRA2, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, and VEGFA; and generate a prediction of multiple sclerosis disease progression by applying a predictive model to the expression levels of the plurality of biomarkers.

Disclosed herein is also a non-transitory computer readable medium comprising instructions that, when executed by a processor, cause the processor to: obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, IL15, NEFL, GFAP, and VEGFA, and optionally comprises at least one or more of: CXCL-13, APLP1, MOG, OPG, VCAN, CDCP1, TNFSF13b, CNTN2, CXCL9, SERPINA9, OPN, CD6, TNFRSF10a, CCL20, COL4A1, GH, IL-12, PRTG, CXCL10, EGF, CXCL11, CFH, TNFSF10, IL18, IL6, TNF, CXCL13, NEFL, CCL20/MIP 3-α, FLRT2; and generate a prediction of multiple sclerosis disease progression by applying a predictive model to the expression levels of the plurality of biomarkers.

In various embodiments, a non-transitory computer readable medium comprises instructions that, when executed by a processor, cause the processor to: obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL; and generate a prediction of multiple sclerosis disease progression by applying a predictive model to the expression levels of the plurality of biomarkers.

In various embodiments, a non-transitory computer readable medium comprising instructions that, when executed by a processor, cause the processor to: obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2; and generate a prediction of multiple sclerosis disease progression by applying a predictive model to the expression levels of the plurality of biomarkers.

In various embodiments, wherein the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

In various embodiments, wherein the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

In various embodiments, a performance of the predictive model is characterized by an AUROC of at least 0.68, at least 0.69, at least 0.70, at least 0.71, at least 0.72, at least 0.73, at least 0.74, at least 0.76, at least 0.77, at least 0.81, at least 0.82, at least 0.83, or at least 0.86.

In various embodiments, the prediction of multiple sclerosis disease progression is a prediction of relapse associated worsening (RAW).

In various embodiments, the prediction of multiple sclerosis disease progression is a prediction of progression independent of relapse activity (PIRA).

In various embodiments, the prediction of multiple sclerosis disease progression is a measure of expanded disability status scale (EDSS) score.

In various embodiments, an expanded disability status scale (EDSS) score less than 6 indicates a mild/moderate MS disease progression and a EDSS score greater than or equal to 6 indicates a severe MS disease progression.

In various embodiments, the prediction of multiple sclerosis disease progression is a measure of a patient determined disease steps (PDDS) score.

In various embodiments, the prediction of multiple sclerosis disease progression is a patient determined disease steps (PDDS) score differentiating between severe and mild/moderate MS disease progression.

In various embodiments, a patient determined disease steps (PDDS) score less than or equal to 4 indicates a mild/moderate MS disease progression and a PDDS score greater than 4 indicates a severe MS disease progression.

In various embodiments, the prediction of multiple sclerosis disease progression is a PRO measurement information system (PROMIS) score.

In various embodiments, the prediction of multiple sclerosis disease progression is a Multiple Sclerosis Rating Scale, Revised (MSRS-R) score.

In various embodiments, the prediction of multiple sclerosis disease progression is a MRI-based volumetric measurement.

In various embodiments, the MRI-based volumetric measurement is one of whole brain atrophy, brain parenchymal fraction (BPF), white matter atrophy, or gray matter atrophy.

In various embodiments, the multiple sclerosis is one of relapsing-remitting multiple sclerosis (RRMS), secondary progressive multiple sclerosis (SPMS), primary-progressive multiple sclerosis (PPMS), progressive relapsing multiple sclerosis (PRMS), or clinically isolated syndrome (CIS).

In various embodiments, the dataset is derived from a sample obtained from the subject.

In various embodiments, the sample is a blood, serum, or plasma sample.

In various embodiments, obtaining or having obtained the dataset comprises performing one or more assays.

In various embodiments, performing one or more assays comprises performing an immunoassay to determine the expression levels of the plurality of biomarkers.

In various embodiments, the immunoassay is a Proximity Extension Assay (PEA) or LUMINEX xMAP Multiplex Assay.

In various embodiments, the methods disclosed herein further comprise administering a therapy to the subject based on the prediction of multiple sclerosis disease progression.

In various embodiments, generating the prediction of multiple sclerosis disease progression by applying the predictive model to the expression levels of the plurality of biomarkers further comprises applying the predictive model to one or more subject attributes of the subject, wherein subject attributes comprise any of age, sex, and disease duration. In various embodiments, generating the prediction of multiple sclerosis disease progression comprises comparing a score outputted by the predictive model to a reference score. In various embodiments, the reference score corresponds to any of: A) an EDSS score; B) a brain parenchymal fraction value; C) a PDDS score; D) a PROMIS score; or E) a MSRS-R score. In various embodiments, the reference score further corresponds to a mild/moderate MS disease progression or a severe MS disease progression.

In various embodiments, the expression levels of the plurality of biomarkers is determined from a test sample obtained from the subject. In various embodiments, the test sample is a blood or serum sample. In various embodiments, the subject has multiple sclerosis, is suspected of having multiple sclerosis, or was previously diagnosed with multiple sclerosis.

In various embodiments, obtaining or having obtained the dataset comprises performing an immunoassay to determine the expression levels of the plurality of biomarkers. In various embodiments, the immunoassay is a Proximity Extension Assay (PEA) or LUMINEX xMAP Multiplex Assay. In various embodiments, performing the immunoassay comprises contacting a test sample with a plurality of reagents comprising antibodies. In various embodiments, the antibodies comprise one of monoclonal and polyclonal antibodies. In various embodiments, the antibodies comprise both monoclonal and polyclonal antibodies. In various embodiments, the method further comprises: selecting a therapy for administering to the subject based on the prediction of multiple sclerosis disease progression. In various embodiments, the method further comprises: determining a therapeutic efficacy of a therapy previously administered to the subject based on the prediction of multiple sclerosis disease progression. In various embodiments, determining the therapeutic efficacy of the therapy comprises comparing the prediction to a prior prediction determined for the subject at a prior timepoint In various embodiments, determining the therapeutic efficacy of the therapy comprises determining that the therapy exhibits efficacy responsive to a difference between the prediction and the prior prediction. In various embodiments, determining the therapeutic efficacy of the therapy comprises determining that the therapy lacks efficacy responsive to a lack of difference between the prediction and the prior prediction.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should be allowed for.

### Example 1: Univariate Analysis to Identify Biomarkers Predictive of MS Disease Progression

A univariate analysis was performed to identify biomarkers informative for predicting MS disease progression. Biomarker express levels of a MS progression patient cohort were obtained and analyzed. Demographics of the MS progression patient cohort is described below in Table 2A.

TABLE 2A
Demographics of Multiple Sclerosis Patients

| | | |
|---|---|---|
| Worsening | | |
| progressive MS | Stable MS | p-value |

| | | |
|---|---|---|
| Samples | 184 | 169 |
| Number of patients | 18 | 19 |

Baseline

| Age (at BL) | 43.8 | | 44.2 | 0.784 |
|---|---|---|---|---|
| RRMS (at BL) | 10 | | 18 | 0.018 |
| PMS (at BL) | 8 | | 1 | |
| EDSS score (at BL) | 4.0 | | 3.0 | 0.065 |
| Monoclonal antibody | 5 | (27.8%) | 5 (26.3) | |
| DMT (BL) | | | | |

Follow-up data

| FU time (years) | 6.5 | | 7.1 | 0.395 |
|---|---|---|---|---|
| EDSS score (last FU) | 6.0 | | 2.5 | <0.001 |
| Number of CDP events | 100% | | 0% | <0.001 |
| | (34 events) | | | |
| Monoclonal antibody | 14 | (77.8) | 3 (15.8) | <0.001 |
| DMT (last FU) | | | | |

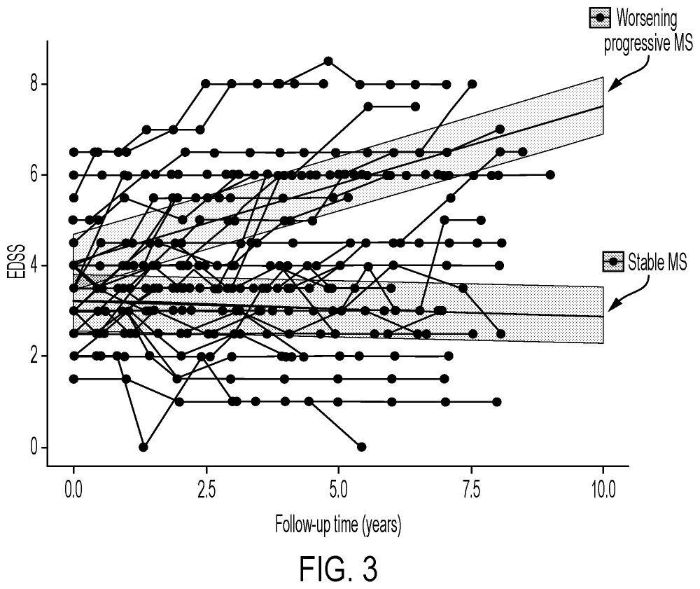

Furthermore, the MS progression (as indicated by increasing EDSS score) of the patients in the cohort are shown below in FIG. 3.

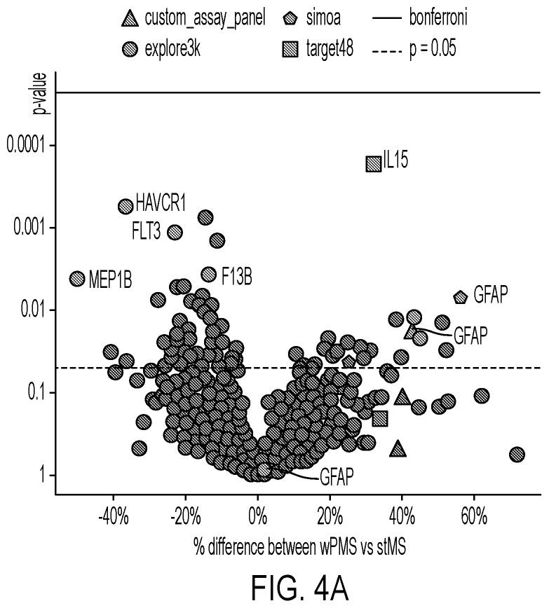

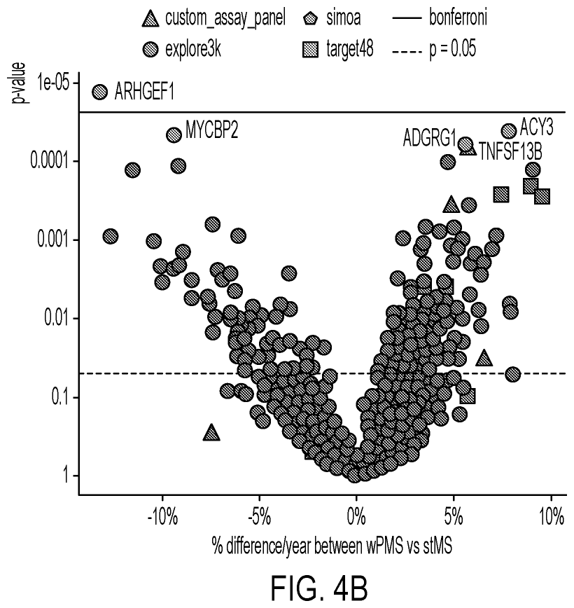

Table 2B below shows the univariate analysis of the expression of various biomarkers from the patient cohort identified in Table 2A. Multiple different proteomic panel technologies were used to analyze biomarker including Single-molecule Array (SIMOA) technology, Olink Target48, Olink Explore 3072, and a Custom Panel assay. The column in Table 2B entitled “p-value (prog vs non-prog)” identifies p-values when comparing expression of the biomarker in patients that experience MS progression (“prog) in comparison to patients that do not experience MS progression (“non prog”). Data is visualized in FIG. 4A-4B.

TABLE 2B
Example univariate analysis of biomarkers (identified by UNIPROT identifiers)

| | | | Est (prog | | | |
|---|---|---|---|---|---|---|
| | | | vs non | | | |
| | | | prog, | | | |
| | | | including | p-value | | |
| Panel | | | slopes | (prog vs | Est | p-value |
| Technology | Biomarker | UNIPROT | 11.2) | non-prog) | (interaction) | (interaction) |
| Simoa | GFAP | P14136 | 1.56 | 0.0071 | 1.01 | 0.4509 |
| Custom Assay | GFAP | P14136 | 1.43 | 0.019 | 1.02 | 0.0857 |
| Custom Assay | TNFSF13B | Q9Y275 | 1.15 | 0.1319 | 1.06 | <0.0001 |
| Explore3K | NEFL | P07196 | 1.18 | 0.0824 | 1.00 | 0.8261 |
| Custom Assay | NEFL | P07196 | 1.25 | 0.0451 | 1.00 | 0.8958 |
| Simoa | NEFL | P07196 | 1.26 | 0.0451 | 1.00 | 0.7978 |
| Explore3K | TNFSF13B | Q9Y275 | 1.12 | 0.3295 | 1.06 | 0.0008 |
| Explore3K | GFAP | P14136 | 1.02 | 0.8122 | 1.00 | 0.7508 |
| target48 | IL15 | P40933 | 1.3223 | 0.0002 | 1.02 | 0.0824 |
| Explore3K | HAVCR1 | Q96D42 | 0.63 | 0.0006 | 1.02 | 0.271 |
| Explore3K | FLT3 | P36888 | 0.77 | 0.0011 | 1.01 | 0.5234 |
| Explore3K | MAN1A2 | O60476 | 0.85 | 0.0008 | 1.02 | 0.0277 |
| Explore3K | IL15 | P40933 | 1.12 | 0.0512 | 1.01 | 0.467 |
| Explore3K | ACY3 | Q96HD9 | 0.83 | 0.1232 | 1.08 | 0 |
| Explore3K | ARHGEF1 | Q92888 | 1.34 | 0.1137 | 0.87 | 0 |
| Explore3K | ADGRG1 | Q9Y653 | 0.97 | 0.7242 | 1.06 | 0.0001 |
| Explore3K | MYCBP2 | O75592 | 1.12 | 0.4195 | 0.91 | 0 |
| Explore3K | ITGB1 | P05556 | 0.88 | 0.016 | 0.98 | 0.0976 |
| Explore3K | CLEC4A | Q9UMR7 | 0.78 | 0.0052 | 0.98 | 0.2493 |
| Explore3K | MEP1B | Q16820 | 0.50 | 0.0041 | 1.04 | 0.0681 |
| Explore3K | F13B | P05160 | 0.86 | 0.0036 | 1.01 | 0.3303 |
| Explore3K | FCN1 | O00602 | 0.72 | 0.0074 | 1.00 | 0.8746 |
| Explore3K | ADCYAP1R1 | P41586 | 0.89 | 0.0015 | 1.01 | 0.0997 |
| Explore3K | LILRA5 | A6NI73 | 0.79 | 0.0051 | 1.02 | 0.0994 |
| Explore3K | HEPH | Q9BQS7 | 0.85 | 0.0067 | 1.02 | 0.0575 |
| Explore3K | CLEC10A | Q8IUN9 | 0.78 | 0.0135 | 1.02 | 0.252 |
| Explore3K | RABEPK | Q7Z6M1 | 0.88 | 0.0474 | 1.00 | 0.9825 |
| Explore3K | FCER2 | P06734 | 0.64 | 0.0448 | 1.00 | 0.9733 |
| Explore3K | TG | P01266 | 1.51 | 0.014 | 0.93 | 0.0006 |
| target48 | CXCL12 | P48061 | 1.1553 | 0.104 | 1.0145 | 0.1734 |
| Explore3K | CA3 | P07451 | 1.25 | 0.1247 | 1.03 | 0.2053 |
| target48 | CXCL10 | P02778 | 1.1979 | 0.2446 | 1.0955 | 0.0003 |
| target48 | CXCL8 | P10145 | 1.1834 | 0.128 | 1.025 | 0.1353 |
| target48 | CCL8 | P80075 | 1.1906 | 0.1736 | 1.0447 | 0.0039 |
| Custom Assay | CNTN2 | Q02246 | 1.15 | 0.1202 | 1.01 | 0.3947 |
| target48 | EGF | P01133 | 1.1447 | 0.2792 | 1.0352 | 0.1737 |
| target48 | CXCL9 | Q07325 | 1.1515 | 0.2692 | 1.0354 | 0.1554 |
| Explore3K | CD22 | P20273 | 0.71 | 0.1309 | 0.99 | 0.6702 |
| Custom Assay | CXCL-13 | O43927 | 1.39 | 0.1181 | 1.02 | 0.4357 |
| target48 | IL17A | Q16552 | 1.3392 | 0.2075 | 1.0569 | 0.0945 |
| target48 | IL7 | P13232 | 1.1975 | 0.1509 | 1.0161 | 0.3341 |
| Explore3K | KLHL41 | O60662 | 1.10 | 0.2501 | 1.02 | 0.2356 |
| Explore3K | KLRC1 | P26715 | 1.15 | 0.167 | 1.01 | 0.6398 |
| Custom Assay | APLP1 | P51693 | 1.13 | 0.1682 | 1.01 | 0.6197 |
| Explore3K | FCRL1 | Q96LA6 | 0.77 | 0.1631 | 1.00 | 0.8871 |
| target48 | IL17C | Q9POM4 | 0.8109 | 0.1346 | 1.0088 | 0.6806 |
| target48 | CXCL11 | O14625 | 1.1008 | 0.4978 | 1.0893 | 0.0002 |
| Custom Assay | CXCL-9 | Q07325 | 1.11 | 0.4221 | 1.04 | 0.1577 |
| Explore3K | KLKB1 | P03952 | 0.94 | 0.1811 | 1.00 | 0.9973 |
| Explore3K | IFNGR2 | P38484 | 1.09 | 0.4123 | 1.06 | 0.0004 |
| Explore3K | CST7 | O76096 | 0.77 | 0.1426 | 1.02 | 0.2713 |
| Explore3K | CFH | P08603 | 0.93 | 0.1472 | 1.01 | 0.2196 |
| target48 | FLT3LG | P49771 | 1.1117 | 0.2428 | 1.003 | 0.8041 |
| Custom Assay | SERPINA9 | Q86WD7 | 1.22 | 0.2815 | 1.01 | 0.6055 |
| Explore3K | SERPINA9 | Q86WD7 | 1.17 | 0.3513 | 1.01 | 0.6387 |
| target48 | TNFSF10 | P50591 | 1.03 | 0.6354 | 1.0246 | 0.0197 |
| target48 | CCL19 | Q99731 | 1.1061 | 0.5678 | 1.0747 | 0.0003 |
| Explore3K | GFRA2 | O00451 | 1.05 | 0.3029 | 1.00 | 0.5246 |
| Custom Assay | COL4A1 | P02462 | 0.87 | 0.2210 | 1.03 | 0.0766 |
| Explore3K | SERPINA3 | P01011 | 1.0 | 0.622 | 1.01 | 0.2288 |
| Explore3K | KIRREL1 | Q96J84 | 1.10 | 0.3137 | 0.99 | 0.3349 |
| Custom Assay | OPN | P10451 | 1.07 | 0.4344 | 1.00 | 0.7343 |
| Custom Assay | CD6 | P30203 | 0.88 | 0.3390 | 1.02 | 0.135 |
| Custom Assay | TNFRSF10A | O00220 | 1.05 | 0.5987 | 1.02 | 0.0976 |
| target48 | LTA | P01374 | 1.0612 | 0.624 | 1.0302 | 0.0077 |
| Custom Assay | MOG | Q16653 | 1.07 | 0.4928 | 1.00 | 0.8261 |
| target48 | IL18 | Q14116 | 0.8791 | 0.2986 | 1.0461 | 0.0038 |
| Explore3K | AMPD3 | Q01432 | 1.26 | 0.0999 | 0.91 | 0.0001 |
| target48 | CCL2 | P13500 | 1.0414 | 0.6527 | 1.0078 | 0.5409 |
| Explore3K | DPEP2 | Q9H4A9 | 1.02 | 0.8252 | 1.02 | 0.1212 |
| target48 | IL6 | P05231 | 0.9277 | 0.7154 | 0.9775 | 0.4792 |
| Explore3K | CFHR5 | Q9BXR6 | 0.95 | 0.3881 | 1.02 | 0.1444 |
| Custom Assay | GH | P01242 | 1.38 | 0.4851 | 0.93 | 0.315 |
| Explore3K | F10 | P00742 | 0.96 | 0.5193 | 1.01 | 0.4549 |
| Explore3K | SERPIND1 | P05546 | 1.04 | 0.6987 | 1.01 | 0.6293 |
| target48 | CSF3 | P09919 | 0.9317 | 0.5959 | 1.017 | 0.3427 |
| target48 | CCL13 | Q99616 | 0.927 | 0.5257 | 1.0303 | 0.0428 |
| Explore3K | PFKFB2 | O60825 | 1.26 | 0.2615 | 0.88 | 0.0001 |
| Custom Assay | CCL20 | P78556 | 0.99 | 0.9587 | 1.07 | 0.0323 |
| target48 | CSF1 | P09603 | 0.9909 | 0.8432 | 1.0187 | 0.0093 |
| Custom Assay | OPG | O00300 | 1.03 | 0.7147 | 0.99 | 0.6556 |
| Explore3K | APOF | Q13790 | 1.01 | 0.8095 | 1.00 | 0.9766 |
| Custom Assay | IL12B | P29460 | 1.01 | 0.9677 | 1.04 | 0.0183 |
| target48 | MMP12 | P39900 | 0.9399 | 0.638 | 1.0298 | 0.074 |
| Explore3K | LMOD1 | P29536 | 1.01 | 0.8731 | 0.98 | 0.1115 |
| Explore3K | RNASE10 | Q5GAN6 | 0.96 | 0.7874 | 1.01 | 0.5522 |
| Explore3K | APCS | P02743 | 0.99 | 0.9435 | 1.02 | 0.106 |
| target48 | MMP1 | P03956 | 1.0089 | 0.9578 | 1.0313 | 0.0129 |
| Explore3K | CCL19 | Q99731 | 0.88 | 0.4621 | 1.09 | 0.0001 |
| Explore3K | CEP20 | Q96NB1 | 1.02 | 0.8194 | 0.98 | 0.1193 |
| Explore3K | NAMPT | P43490 | 1.02 | 0.7407 | 0.98 | 0.2134 |
| Custom Assay | VCAN | P13611 | 0.98 | 0.6925 | 1.01 | 0.1295 |
| target48 | OLR1 | P78380 | 1.0532 | 0.7847 | 0.979 | 0.415 |
| Custom Assay | PRTG | Q2VWP7 | 1.01 | 0.8735 | 1.00 | 0.868 |
| Explore3K | ADAMTSL2 | Q86TH1 | 0.96 | 0.6046 | 1.05 | 0.0001 |
| Custom Assay | CDCP1 | Q9H5V8 | 0.95 | 0.5088 | 1.05 | 0.0003 |
| target48 | TNF | P01375 | 0.9601 | 0.6874 | 1.0351 | 0.0038 |
| target48 | VEGFA | P15692 | 0.9659 | 0.8419 | 1.0326 | 0.0434 |
| Custom Assay | FLRT2 | O43155 | 0.99 | 0.9237 | 1.01 | 0.3694 |

### Example 2: Univariate Analysis to Identify Biomarkers Predictive of MS Disease Progression

A univariate analysis was performed to identify serum biomarkers prognostic of gray and white matter atrophy, which is informative for predicting MS disease progression. 37 MS patients from 2 phenotypically extreme MS groups followed yearly were available (Swiss MS Cohort Study, SMSC): (1) Worsening progressive MS (wPMS): Samples from 18 patients; median follow-up of 5.25 years between baseline visit and last MRI scan; median EDSS of 4.0 at baseline and 5.75 at last visit; no relapses during follow-up; (2) stable MS: Samples from 19 patients; 6.0 years median follow-up between baseline visit and last MRI scan; median EDSS from 3.0 at baseline visit to 2.5 at last visit. wPMS and stMS were matched by age, disease duration, EDSS and T2 lesion volume at baseline. See Table 3.

TABLE 3
Demographics

| | Stable MS | wPMS |
|---|---|---|
| Number of patients | 19 | 18 |
| Number of samples = MRIs | 105 | 93 |
| Median samples/patient | 5.0 | 5.5 |

Baseline visit data

| | | |
|---|---|---|
| Sex (Number women, %) | (12, 63.2) | (11, 61.1) |
| Median age (years) | 44.19 | 43.82 |
| Median BMI | 27.54 | 25.29 |
| Median EDSS | 3 | 4 |
| Median disease duration | 9.37 | 13.7 |
| DMT (Number, %) | | |
| Platform | (4, 21.1) | (0, 0.0) |
| Untreated | (3, 15.8) | (6, 33.3) |
| Monoclonal antibodies | (4, 21.1) | (6, 33.3) |
| Orals | (8, 42.1) | (6, 33.3) |
| MRI total intracranial | 15.19 | 14.44 |
| volume (TIV) (mL) | | |

Follow-up data

| | | |
|---|---|---|
| Median follow-up time | 6.02 | 5.25 |
| at last MRI | | |
| Median EDSS at last visit | 2.5 | 5.75 |
| Total Relapses | 0 | 0 |

Samples were analyzed with 3 assays: Olink Explore 3072, Target 48, and the Octave Custom Assay Panel. GFAP and NEFL were also measured by Simoa assays. Brain MRI scans were performed annually in the SMSC. A standardized imaging protocol was applied across centers including a 3D MPRAGE and FLAIR sequence. T1w images were lesion-filled using FSL and segmented by SPM12 to compute gray (GMV) and white matter volume (GMV). Two linear mixed-effects models (see equation below) were run on the combined cohorts (1) and (2).

$$log ( white\_or \_gray \_matter \_volume ) \sim mri\_tiv + age\_at \_baseline + sex + disease\_duration \_at \_baseline + biomarker\_value \_at \_baseline + followup\_time \_years ++ biomarker\_value \_at \_baseline \times followup\_time \_years + patient\_id * + mri\_scanner \_change *$$

Equation for linear mixed-effects models for white and gray matter volume. Baseline=date of serum collection. Follow-up time calculated from baseline serum collection date and MRI date. Bold=contrast of interest, *=random effects.

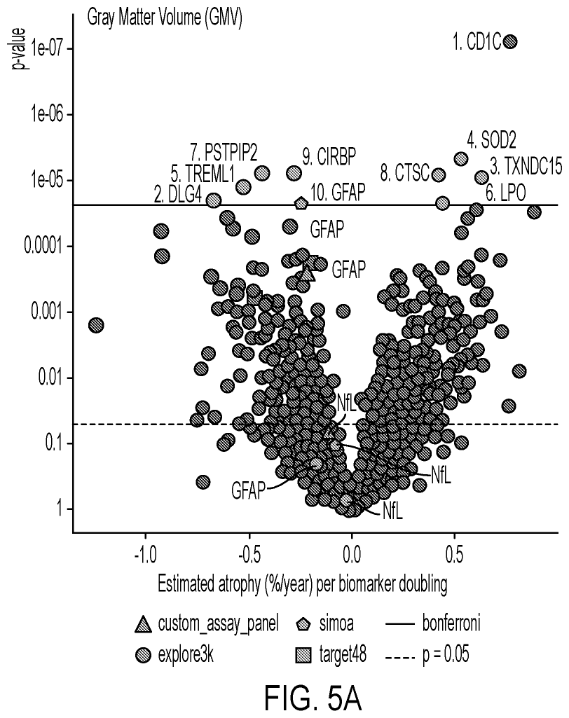

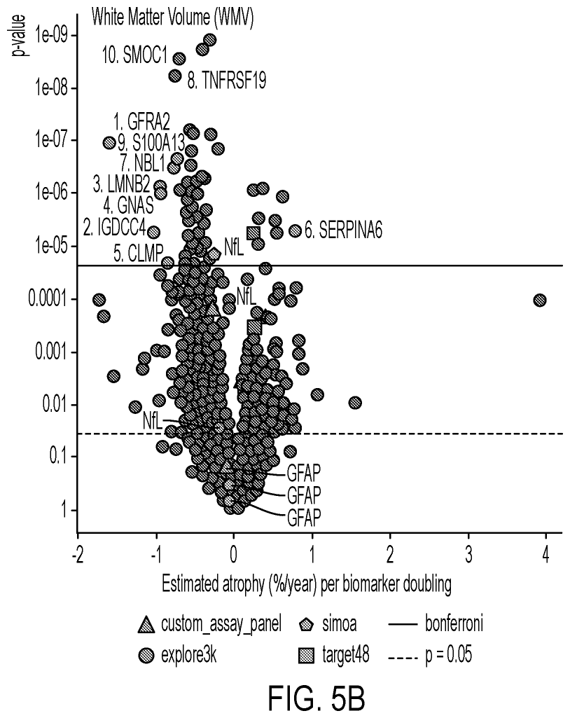

The interaction between log of the baseline biomarker concentration and FU time was the contrast of interest. Proteins were ranked by effect size and compared to, GFAPSimoa for GMV and NEFLSimoa for WMV. Data is visualized in FIG. 5A-5B. Each doubling of baseline GFAPSimoa led to an additional loss of GMV (−0.25%/y [−0.36, −0.14], p<0.0001) but not WMV (−0.07% [−0.20, 0.07], p=0.35), while doubling of baseline NEFLSimoa resulted in additional loss of WMV (−0.25%/y [−0.36, −0.14], p<0.0001) but not GMV (−0.08%/y [−0.18, 0.02], p=0.10).

GMV analysis identified 9 proteins that scored better than GFAPSimoa and passed Bonferroni significance. The top two proteins were CD1C, a dendritic cell marker, baseline doubling leading to (0.77%/y [0.50, 1.04], p=7.63e-08), and DLG4, a synaptic protein, baseline doubling leading to (−0.67%/y [−0.96, −0.37], p=1.99e-05). The top five effect-size proteins and GFAP are in Table 2.

WMV analysis identified 52 proteins that scored better than NEFLSimoa and passed Bonferroni significance. The top two proteins were GFRA2, a glial cell neurotrophic factor receptor, baseline doubling leading to (−1.59%/y [−2.15, −1.04], p=1.10e-07) and IGDCC4, an immunoglobulin superfamily member, baseline doubling leading to (−1.04%/y [−1.47, −0.61], p=5.74e-06). The top five effect-size proteins and NEFL are in Table 4.

TABLE 4

| | | |
|---|---|---|
| Assay (* = explore3k | %/year change due to | |
| [ ] = Simoa) | doubling (95% CI) | P-val |

GMV

| | | | |
|---|---|---|---|
| CD1C* | 0.77 | (0.5, 1.04) | 7.63E−08 |
| DLG4* | −0.67 | (−0.96, −0.37) | 1.99E−05 |
| TXNDC15* | 0.63 | (0.36, 0.9) | 8.65E−06 |
| SOD2* | 0.53 | (0.31, 0.75) | 4.41E−06 |
| TREML1* | −0.53 | (−0.75, −0.3) | 1.25E−05 |
| [GFAP] | −0.25 | (−0.36, −0.14) | 2.23E−05 |

WMV

| | | | |
|---|---|---|---|
| GFRA2* | −1.59 | (−2.15, −1.04) | 1.10E−07 |
| IGDCC4* | −1.04 | (−1.47, −0.61) | 5.74E−06 |
| LMNB2* | −0.95 | (−1.31, −0.59) | 7.42E−07 |
| GNAS* | −0.94 | (−1.3, −0.58) | 9.26E−07 |
| CLMP* | −0.85 | (−1.23, −0.47) | 2.18E−05 |
| [NEFL] | −0.25 | (−0.36, −0.14) | 1.47E−05 |

### Example 3: Univariate Analysis to Identify Biomarkers Predictive of MS Disease Progression

A univariate analysis was performed to identify blood biomarkers capturing i) EDSS progression (independent of relapse activity) and ii) gray and white matter brain atrophy using well-characterized longitudinal cohorts with and without severe disease progression.

Serum samples from 2 phenotypically extreme MS groups followed yearly were analyzed (Swiss MS Cohort Study): (1) Worsening progressive MS (wPMS): 184 samples from 18 patients; median follow-up of 6.5 years; median EDSS of 4.0 at baseline (BL) and 6.0 at last visit; no relapses during follow-up (as summarized in Table 5); (2) stable MS (stMS): 169 samples from 19 patients; 7.1 years median follow-up; median EDSS from 3.0 to 2.5. wPMS and stMS were matched by age, disease duration, EDSS and T2 lesion volume at BL (as summarized in Table 3). Samples were analyzed with 3 Olink assays: Explore 3072, Target 48, and the Octave Custom Assay Panel, and 2 Simoa assays for GFAP and NEFL. Brain MRI scans were performed annually using a standardized imaging protocol. Single protein linear-mixed-effects models for each endpoint were run on the combined cohorts (1) and (2). Variables of interest and covariates, including patient demographics and clinical characteristics, were dependent on the model. Proteins were ranked by p-value and effect size.

TABLE 5
Demographic information of the full cohort.

| | | | | Worsening | | |
|---|---|---|---|---|---|---|
| | | Stable MS | | progressive MS | | p-value |
| | Samples (n) | 169 | | 184 | | |
| | Number of patients | 19 | | 18 | | |
| Baseline visit data | | | | | | |
| | Age (years) | 44.2 | [39.5-49.2] | 43.8 | [40.9-53.8] | 0.784 |
| | Disease category at | | | | | 0.018 |
| | study entry (%) | | | | | |
| | RRMS | 18 | (94.7) | 10 | (55.6) | |
| | PMS | 1 | (5.3) | 8 | (44.4) | |
| | EDSS score | 3.0 | [2.5-3.8] | 4.0 | [3.1-4.4] | 0.65 |
| Follow-up data | | | | | | |
| | Number of samples | 9 | [8, 10] | 10 | [9, 12.5] | 0.097 |
| | per patient | | | | | |
| | FU time (years) | 7.1 [5.7-8.0] | | 6.5 [5.2-7.7] | | 0.395 |
| | (range) | [4.1-9.0] | | [2.7-8.5] | | |
| | EDSS score at last | 2.5 | [2.0-3.8] | 6.0 | [5.6-6.9] | <0.001 |
| | sampling | | | | | |
| | Number of confirmed | | | | | <0.001 |
| | disease progression | | | | | |
| | events (%) | | | | | |
| | 0 | 19 | (100.0) | 0 | (0.0) | |
| | 1 | 0 | (0.0) | 6 | (33.3) | |
| | 2 | 0 | (0.0) | 8 | (44.4) | |
| | 3 | 0 | (0.0) | 4 | (22.2) | |

The following formulas were used to model the relationship between progressor status (clinical model), gray matter, and white matter atrophy with protein concentration:

$$Clinical Model : log ( protein concentration ) \sim 1 + age at baseline + disease duration at baseline + BMI + sex + DMT category [ Orals , Platform , Monoclonal , None ] + EDSS + progressor status + followup time + progressor status : followup time + patient id *$$

$$\begin{matrix}MRI Models : log ( white or gray matter volume ) \sim 1 + MRITIV + age at baseline + sex + disease duration at baseline + protein concentration at baseline + follow up time + protein concentration at baseline : follow up time + patient id * MRI scanner change * \\ *= random effect \\ Bold text = coefficient of interest\end{matrix}$$

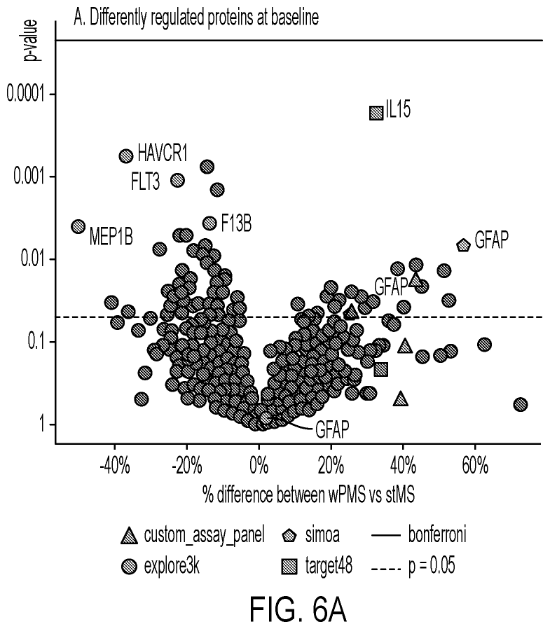

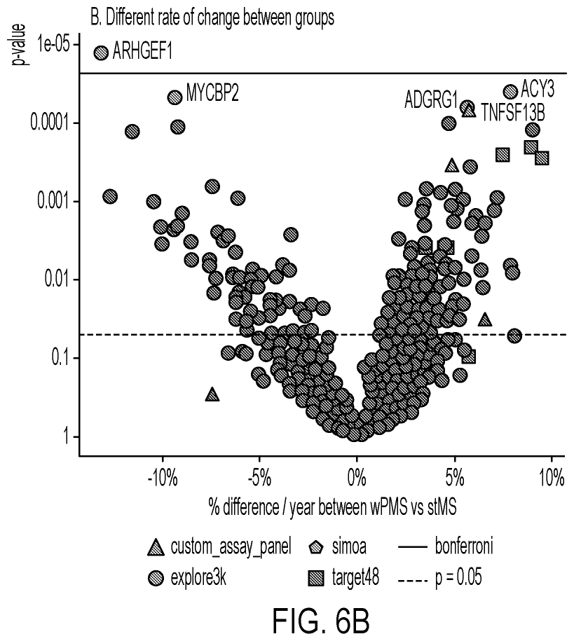

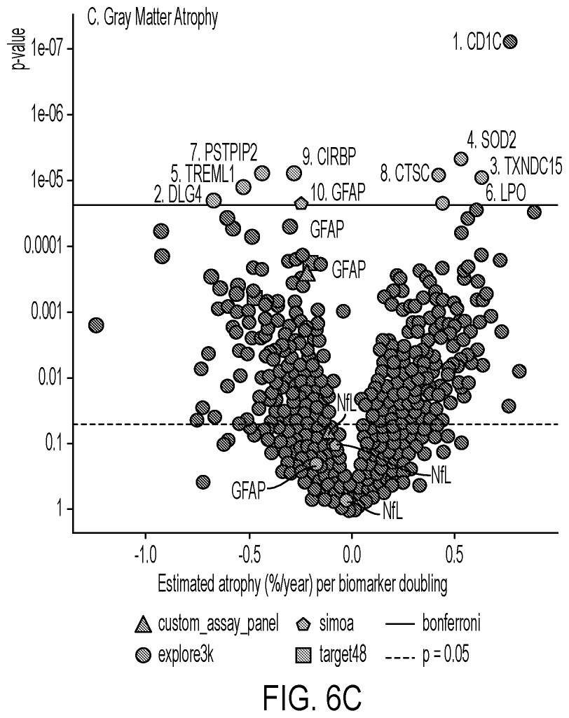

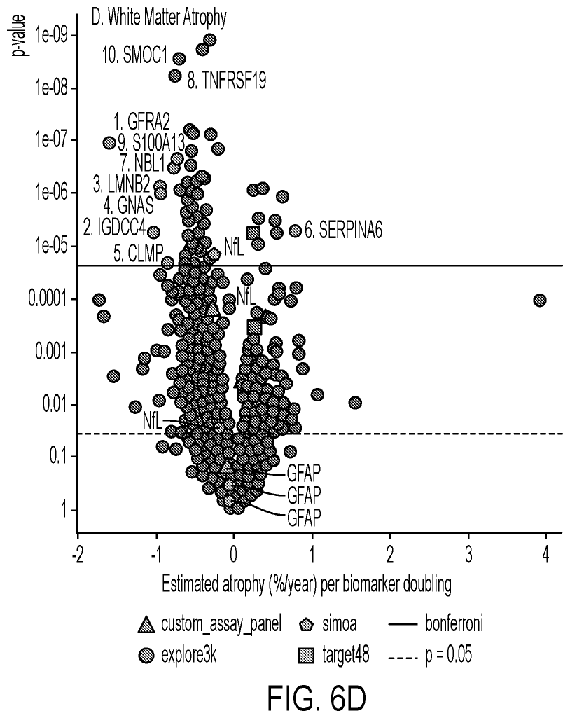

Serum protein differences were found between wPMS and stMS, with several detected at higher significance than GFAP or NEFL, as visualized in FIG. 6A-6D. IL15, a proinflammatory cytokine, was the strongest differentiator at the group level and was increased by 32% [15, 51](p=1.17e-04) in wPMS. ARHGEF1, a nucleotide exchange factor, showed the largest change over time and decreased by 13%/y [18, 8](p=1.32e-05) in wPMS. CD1C, a dendritic cell marker, was the top protein for gray matter volume loss with baseline doubling leading to 0.77%/y [0.50, 1.04](p=7.63e-08). GFRA2, a glial cell neurotrophic factor receptor, was the top protein for white matter volume loss with baseline doubling leading to −1.59%/y [−2.15, −1.04](p=1.10e-07).

### Example 4: Multivariate Analysis to Identify Biomarkers Predictive of MS Disease Progression

Biomarkers were selected for a multivariate custom panel according to their correlation with the progressor status (binary classification into progressor/non-progressor) as defined by PIRA.

Multivariate analyses of biomarker panels were conducted across the different human clinical studies according to the methods described in Examples 1-3. In particular biomarkers were categorized into different tiers (e.g., Tier 1, Tier 2, and Tier 3). Biomarker panels were constructed from one or more tiers (e.g., Tier 1 alone, tier 2 alone, or tier 3 alone).

Linear regression models (with L1 regularization) were trained and cross-validated on the Basel Aim C Extreme Phenotypes of Progression cohort and dataset.

Disease progressor status as defined by PIRA (Progression Independent of Relapse Activity) was the clinical outcome of interest, predicted by these models. The label was provided by clinicians at the University Hospital Basel. The same model-building strategy was re-deployed on progressively larger subsets/tiers of markers on the panel to report Area Under the Curve (AUC). Statistical measures of the multivariate analysis AUC are shown below.

Generally, biomarker panels that employed biomarkers from each of tier 1, tier 2, and tier 3 corresponded to predictive models that exhibited improved predictive capacity across the different disease activity endpoints (e.g., subtle disease activity, general disease activity, extreme disease activity, annualized relapse rate, or disease state). Specifically, the AUC across these different disease activity endpoints ranged from 0.50 up to 0.87. Biomarker panels employing biomarkers from only tier 1 achieved AUC values across the different disease endpoints that ranged from 0.63 up to 0.81. Biomarker panels employing biomarkers from only tier 2 achieved AUC values across the different disease endpoints that ranged from 0.68 up to 0.86. Biomarker panels employing biomarkers from only tier 1 achieved AUC values across the different disease endpoints that ranged from 0.50 up to 0.87. Data is shown in Tables 6-8.

TABLE 6
Tier 1 Biomarker Set Multivariate Analysis.

| Biomarker Set | AUROC |
|---|---|
| GFRA2, GFAP | 0.78 |
| GFRA2, GFAP, TREML1 | 0.78 |
| GFRA2, GFAP, TREML1, CLMP | 0.78 |
| GFRA2, GFAP, TREML1, CLMP, SOD2 | 0.78 |
| GFRA2, GFAP, TREML1, CLMP, SOD2, IGDCC4 | 0.78 |
| GFRA2, GFAP, TREML1, CLMP, SOD2, IGDCC4, GNAS | 0.74 |
| GFRA2, GFAP, TREML1, CLMP, SOD2, IGDCC4, GNAS, NEFL | 0.70 |
| GFRA2, GFAP, TREML1, CLMP, SOD2, IGDCC4, GNAS, NEFL, DLG4 | 0.67 |
| GFRA2, GFAP, TREML1, CLMP, SOD2, IGDCC4, GNAS, NEFL, DLG4, | 0.67 |
| TXNDC15 | |
| GFRA2, GFAP, TREML1, CLMP, SOD2, IGDCC4, GNAS, NEFL, DLG4, | 0.66 |
| TXNDC15, CD1C | |
| GFRA2, GFAP, TREML1, CLMP, SOD2, IGDCC4, GNAS, NEFL, DLG4, | 0.63 |
| TXNDC15, CD1C, IL15 | |
| GFRA2, GFAP, TREML1, CLMP, SOD2, IGDCC4, GNAS, NEFL, DLG4, | 0.69 |
| TXNDC15, CD1C, IL15, FLT3 | |
| GFRA2, GFAP, TREML1, CLMP, SOD2, IGDCC4, GNAS, NEFL, DLG4, | 0.75 |
| TXNDC15, CD1C, IL15, FLT3, MEP1B | |
| GFRA2, GFAP, TREML1, CLMP, SOD2, IGDCC4, GNAS, NEFL, DLG4, | 0.81 |
| TXNDC15, CD1C, IL15, FLT3, MEP1B, HAVCR1 | |
| GFRA2, GFAP, TREML1, CLMP, SOD2, IGDCC4, GNAS, NEFL, DLG4, | 0.81 |
| TXNDC15, CD1C, IL15, FLT3, MEP1B, HAVCR1, F13B | |

TABLE 7
Tier 2 Biomarker Set Multivariate Analysis.

| Biomarker Set | AUROC |
|---|---|
| TNFSF13B, GNAS | 0.74 |
| TNFSF13B, GNAS, NEFL | 0.86 |
| TNFSF13B, GNAS, NEFL, GFAP | 0.82 |
| TNFSF13B, GNAS, NEFL, GFAP, GFRA2 | 0.82 |
| TNFSF13B, GNAS, NEFL, GFAP, GFRA2, IGDCC4 | 0.82 |
| TNFSF13B, GNAS, NEFL, GFAP, GFRA2, IGDCC4, CLMP | 0.83 |
| TNFSF13B, GNAS, NEFL, GFAP, GFRA2, IGDCC4, CLMP, DLG4 | 0.81 |
| TNFSF13B, GNAS, NEFL, GFAP, GFRA2, IGDCC4, CLMP, DLG4, | 0.81 |
| SERPINA9 | |
| TNFSF13B, GNAS, NEFL, GFAP, GFRA2, IGDCC4, CLMP, DLG4, | 0.77 |
| SERPINA9, OPG | |
| TNFSF13B, GNAS, NEFL, GFAP, GFRA2, IGDCC4, CLMP, DLG4, | 0.76 |
| SERPINA9, OPG, SOD2 | |
| TNFSF13B, GNAS, NEFL, GFAP, GFRA2, IGDCC4, CLMP, DLG4, | 0.76 |
| SERPINA9, OPG, SOD2, CXCL13 | |
| TNFSF13B, GNAS, NEFL, GFAP, GFRA2, IGDCC4, CLMP, DLG4, | 0.74 |
| SERPINA9, OPG, SOD2, CXCL13, CD6 | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1 | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1, OPN | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1, OPN, PRTG | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1, OPN, PRTG, CCL20 | |
| TNFSF13B, GNAS, NEFL, GFAP, GFRA2, IGDCC4, CLMP, DLG4, | 0.74 |
| SERPINA9, OPG, SOD2, CXCL13, CD6, CDCP1, OPN, PRTG, CCL20, | |
| FLT3 | |
| TNFSF13B, GNAS, NEFL, GFAP, GFRA2, IGDCC4, CLMP, DLG4, | 0.73 |
| SERPINA9, OPG, SOD2, CXCL13, CD6, CDCP1, OPN, PRTG, CCL20, | |
| FLT3, CNTN2 | |
| TNFSF13B, GNAS, NEFL, GFAP, GFRA2, IGDCC4, CLMP, DLG4, | 0.73 |
| SERPINA9, OPG, SOD2, CXCL13, CD6, CDCP1, OPN, PRTG, CCL20, | |
| FLT3, CNTN2, TXNDC15 | |
| TNFSF13B, GNAS, NEFL, GFAP, GFRA2, IGDCC4, CLMP, DLG4, | 0.71 |
| SERPINA9, OPG, SOD2, CXCL13, CD6, CDCP1, OPN, PRTG, CCL20, | |
| FLT3, CNTN2, TXNDC15, IL15 | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1, OPN, PRTG, CCL20, | |
| FLT3, CNTN2, TXNDC15, IL15, IL12B | |
| TNFSF13B, GNAS, NEFL, GFAP, GFRA2, IGDCC4, CLMP, DLG4, | 0.71 |
| SERPINA9, OPG, SOD2, CXCL13, CD6, CDCP1, OPN, PRTG, CCL20, | |
| FLT3, CNTN2, TXNDC15, IL15, IL12B, APLP1 | |
| TNFSF13B, GNAS, NEFL, GFAP, GFRA2, IGDCC4, CLMP, DLG4, | 0.71 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2 | |
| TNFSF13B, GNAS, NEFL, GFAP, GFRA2, IGDCC4, CLMP, DLG4, | 0.69 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C | |
| TNFSF13B, GNAS, NEFL, GFAP, GFRA2, IGDCC4, CLMP, DLG4, | 0.68 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, TREML1 | |
| TNFSF13B, GNAS, NEFL, GFAP, GFRA2, IGDCC4, CLMP, DLG4, | 0.71 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, | |
| TREML1, MEP1B | |
| TNFSF13B, GNAS, NEFL, GFAP, GFRA2, IGDCC4, CLMP, DLG4, | 0.71 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, | |
| TREML1, MEP1B, TNFRSF10A | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, | |
| TREML1, MEP1B, TNFRSF10A, MOG | |
| TNFSF13B, GNAS, NEFL, GFAP, GFRA2, IGDCC4, CLMP, DLG4, | 0.72 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, | |
| TREML1, MEP1B, TNFRSF10A, MOG, CXCL9 | |
| TNFSF13B, GNAS, NEFL, GFAP, GFRA2, IGDCC4, CLMP, DLG4, | 0.70 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, | |
| TREML1, MEP1B, TNFRSF10A, MOG, CXCL9, HAVCR1 | |
| TNFSF13B, GNAS, NEFL, GFAP, GFRA2, IGDCC4, CLMP, DLG4, | 0.76 |
| SERPINA9, OPG, SOD2, CXCL13, CD6, CDCP1, OPN, PRTG, CCL20, | |
| FLT3, CNTN2, TXNDC15, IL15, IL12B, APLP1, FLRT2, CD1C, | |
| TREML1, MEP1B, TNFRSF10A, MOG, CXCL9, HAVCR1, F13B | |

TABLE 8
Tier 3 Biomarker Set Multivariate Analysis.

| Biomarker Set | AUROC |
|---|---|
| SERPIND1, KLRC1 | 0.51 |
| SERPIND1, KLRC1, CLMP | 0.50 |
| SERPIND1, KLRC1, CLMP, GFRA2 | 0.64 |
| SERPIND1, KLRC1, CLMP, GFRA2, GFAP | 0.79 |
| SERPIND1, KLRC1, CLMP, GFRA2, GFAP, TG | 0.75 |
| SERPIND1, KLRC1, CLMP, GFRA2, GFAP, TG, IL17A | 0.79 |
| SERPIND1, KLRC1, CLMP, GFRA2, GFAP, TG, IL17A, OLR1 | 0.80 |
| SERPIND1, KLRC1, CLMP, GFRA2, GFAP, TG, IL17A, OLR1, CA3 | 0.87 |
| SERPIND1, KLRC1, CLMP, GFRA2, GFAP, TG, IL17A, OLR1, CA3, | 0.87 |
| SERPINA3 | |
| SERPIND1, KLRC1, CLMP, GFRA2, GFAP, TG, IL17A, OLR1, CA3, | 0.87 |
| SERPINA3, GNAS | |
| SERPIND1, KLRC1, CLMP, GFRA2, GFAP, TG, IL17A, OLR1, CA3, | 0.86 |
| SERPINA3, GNAS, KLHL41 | |
| SERPIND1, KLRC1, CLMP, GFRA2, GFAP, TG, IL17A, OLR1, CA3, | 0.86 |
| SERPINA3, GNAS, KLHL41, IFNGR2 | |
| SERPIND1, KLRC1, CLMP, GFRA2, GFAP, TG, IL17A, OLR1, CA3, | 0.87 |
| SERPINA3, GNAS, KLHL41, IFNGR2, ADGRG1 | |
| SERPIND1, KLRC1, CLMP, GFRA2, GFAP, TG, IL17A, OLR1, CA3, | 0.87 |
| SERPINA3, GNAS, KLHL41, IFNGR2, ADGRG1, CXCL8 | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF | |
| SERPIND1, KLRC1, CLMP, GFRA2, GFAP, TG, IL17A, OLR1, CA3, | 0.74 |
| SERPINA3, GNAS, KLHL41, IFNGR2, ADGRG1, CXCL8, APOF, MMP12 | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL, SOD2 | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL, SOD2, CXCL12 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2 | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL, SOD2, CXCL12, DPEP2, FCRL2 | |
| SERPIND1, KLRC1, CLMP, GFRA2, GFAP, TG, IL17A, OLR1, CA3, | 0.79 |
| SERPINA3, GNAS, KLHL41, IFNGR2, ADGRG1, CXCL8, APOF, MMP12, | |
| NEFL, SOD2, CXCL12, DPEP2, FCRL2, FLT3LG | |
| SERPIND1, KLRC1, CLMP, GFRA2, GFAP, TG, IL17A, OLR1, CA3, | 0.83 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1 | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1 | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1 | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3 | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10 | |
| SERPIND1, KLRC1, CLMP, GFRA2, GFAP, TG, IL17A, OLR1, CA3, | 0.83 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4 | |
| SERPIND1, KLRC1, CLMP, GFRA2, GFAP, TG, IL17A, OLR1, CA3, | 0.79 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1 | |
| SERPIND1, KLRC1, CLMP, GFRA2, GFAP, TG, IL17A, OLR1, CA3, | 0.79 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10 | |
| SERPIND1, KLRC1, CLMP, GFRA2, GFAP, TG, IL17A, OLR1, CA3, | 0.79 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1 | |
| SERPIND1, KLRC1, CLMP, GFRA2, GFAP, TG, IL17A, OLR1, CA3, | 0.76 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20 | |
| SERPIND1, KLRC1, CLMP, GFRA2, GFAP, TG, IL17A, OLR1, CA3, | 0.76 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT | |
| SERPIND1, KLRC1, CLMP, GFRA2, GFAP, TG, IL17A, OLR1, CA3, | 0.74 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA | |
| SERPIND1, KLRC1, CLMP, GFRA2, GFAP, TG, IL17A, OLR1, CA3, | 0.71 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15 | |
| SERPIND1, KLRC1, CLMP, GFRA2, GFAP, TG, IL17A, OLR1, CA3, | 0.72 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7 | |
| SERPIND1, KLRC1, CLMP, GFRA2, GFAP, TG, IL17A, OLR1, CA3, | 0.71 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C | |
| SERPIND1, KLRC1, CLMP, GFRA2, GFAP, TG, IL17A, OLR1, CA3, | 0.71 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3 | |
| SERPIND1, KLRC1, CLMP, GFRA2, GFAP, TG, IL17A, OLR1, CA3, | 0.70 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1 | |
| SERPIND1, KLRC1, CLMP, GFRA2, GFAP, TG, IL17A, OLR1, CA3, | 0.70 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15 | |
| SERPIND1, KLRC1, CLMP, GFRA2, GFAP, TG, IL17A, OLR1, CA3, | 0.70 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7 | |
| SERPIND1, KLRC1, CLMP, GFRA2, GFAP, TG, IL17A, OLR1, CA3, | 0.70 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4 | |
| SERPIND1, KLRC1, CLMP, GFRA2, GFAP, TG, IL17A, OLR1, CA3, | 0.73 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22 | |
| SERPIND1, KLRC1, CLMP, GFRA2, GFAP, TG, IL17A, OLR1, CA3, | 0.73 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8 | |
| SERPIND1, KLRC1, CLMP, GFRA2, GFAP, TG, IL17A, OLR1, CA3, | 0.73 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH | |
| SERPIND1, KLRC1, CLMP, GFRA2, GFAP, TG, IL17A, OLR1, CA3, | 0.73 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2 | |
| SERPIND1, KLRC1, CLMP, GFRA2, GFAP, TG, IL17A, OLR1, CA3, | 0.77 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1 | |
| SERPIND1, KLRC1, CLMP, GFRA2, GFAP, TG, IL17A, OLR1, CA3, | 0.72 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C | |
| SERPIND1, KLRC1, CLMP, GFRA2, GFAP, TG, IL17A, OLR1, CA3, | 0.73 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19 | |
| SERPIND1, KLRC1, CLMP, GFRA2, GFAP, TG, IL17A, OLR1, CA3, | 0.74 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B | |
| SERPIND1, KLRC1, CLMP, GFRA2, GFAP, TG, IL17A, OLR1, CA3, | 0.76 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA | |
| SERPIND1, KLRC1, CLMP, GFRA2, GFAP, TG, IL17A, OLR1, CA3, | 0.74 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3 | |
| SERPIND1, KLRC1, CLMP, GFRA2, GFAP, TG, IL17A, OLR1, CA3, | 0.74 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B | |
| SERPIND1, KLRC1, CLMP, GFRA2, GFAP, TG, IL17A, OLR1, CA3, | 0.74 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1 | |
| SERPIND1, KLRC1, CLMP, GFRA2, GFAP, TG, IL17A, OLR1, CA3, | 0.75 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3 | |
| SERPIND1, KLRC1, CLMP, GFRA2, GFAP, TG, IL17A, OLR1, CA3, | 0.75 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1 | |
| SERPIND1, KLRC1, CLMP, GFRA2, GFAP, TG, IL17A, OLR1, CA3, | 0.76 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2 | |
| SERPIND1, KLRC1, CLMP, GFRA2, GFAP, TG, IL17A, OLR1, CA3, | 0.76 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2 | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2, | |
| ADAMTSL2 | |
| SERPIND1, KLRC1, CLMP, GFRA2, GFAP, TG, IL17A, OLR1, CA3, | 0.81 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2, | |
| ADAMTSL2, FCER2 | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2, | |
| ADAMTSL2, FCER2, LMNB2 | |
| SERPIND1, KLRC1, CLMP, GFRA2, GFAP, TG, IL17A, OLR1, CA3, | 0.82 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2, | |
| ADAMTSL2, FCER2, LMNB2, RABEPK | |
| SERPIND1, KLRC1, CLMP, GFRA2, GFAP, TG, IL17A, OLR1, CA3, | 0.83 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2, | |
| ADAMTSL2, FCER2, LMNB2, RABEPK, HAVCR1 | |
| SERPIND1, KLRC1, CLMP, GFRA2, GFAP, TG, IL17A, OLR1, CA3, | 0.80 |
| SERPINA3, GNAS, KLHL41, IFNGR2, ADGRG1, CXCL8, APOF, MMP12, | |
| NEFL, SOD2, CXCL12, DPEP2, FCRL2, FLT3LG, ITGB1, ARHGEF1, | |
| KIRREL1, AMPD3, CFHR5, F10, CCL13, CSF1, PFKFB2, IGDCC4, | |
| TREML1, RNASE10, MMP1, CEP20, NAMPT, VEGFA, TXNDC15, CST7, | |
| CD1C, ACY3, FCRL1, IL15, IL7, DLG4, CD22, CCL8, HEPH, MYCBP2, | |
| FCN1, IL17C, CCL19, F13B, LTA, CSF3, MEP1B, KLKB1, FLT3, | |
| ADCYAP1R1, CCL2, CLEC10A, LMOD1, CLEC4A, MAN1A2, | |
| ADAMTSL2, FCER2, LMNB2, RABEPK, HAVCR1, LILRA5 | |

These results demonstrate minimal sets of predictive biomarkers that are capable of predicting in a linear regression model for disability (i.e. disease progression status at the time of blood draw).

1. A method for predicting multiple sclerosis disease progression in a subject, the method comprising:
obtaining or having obtained a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFAP, NEFL, CXCL-13, APLP1, MOG, OPG, VCAN, CDCP1, TNFSF13b, CNTN2, CXCL9, SERPINA9, OPN, CD6, TNFRSF10a, CCL20, FLRT2, COL4A1, GH, IL-12, PRTG, CXCL10, IL15, EGF, CXCL11, CFH, TNFSF10, IL18, IL6, TNF, HAVCR1, FLT3, MAN1A2, ACY3, ARHGEF1, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, GFRA2, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, and VEGFA; and
generating a prediction of multiple sclerosis disease progression by applying a predictive model to the expression levels of the plurality of biomarkers.

2. The method of claim 1, wherein the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, and NEFL.

3. The method of claim 2, wherein the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and GFAP.

4. The method of claim 2, wherein the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, and NEFL.

5. The method of claim 2, wherein a performance of the predictive model is characterized by an AUROC of at least 0.63, at least 0.66, at least 0.67, at least 0.69, at least 0.70, at least 0.74, at least 0.75, at least 0.78, or at least 0.81.

6. The method of claim 1, wherein the plurality of biomarkers comprises two or more biomarkers selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

7. The method of claim 6, wherein the plurality of biomarkers comprises NEFL and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, GFAP, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

8. The method of claim 6, wherein the plurality of biomarkers comprises GFAP and at least one biomarker selected from CD1C, DLG4, TXNDC15, SOD2, TREML1, IGDCC4, LMNB2, GNAS, CLMP, GFRA2, HAVCR1, FLT3, MEP1B, F13B, IL15, NEFL, CXCL-13, APLP1, MOG, OPG, CDCP1, TNFSF13b, CNTN2, CXCL9, SERPINA9, OPN, CD6, TNFRSF10a, CCL20, IL-12, PRTG, and FLRT2.

9. The method of claim 1, wherein a performance of the predictive model is characterized by an AUROC of at least 0.68, at least 0.69, at least 0.70, at least 0.71, at least 0.72, at least 0.73, at least 0.74, at least 0.76, at least 0.77, at least 0.81, at least 0.82, at least 0.83, or at least 0.86.

10. The method of claim 1, wherein the plurality of biomarkers comprises two or more biomarkers selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, GFAP, and NEFL.

11. The method of claim 10, wherein the plurality of biomarkers comprises NEFL and at least one biomarker selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, and GFAP.

12. The method of claim 10, wherein the plurality of biomarkers comprises GFAP and at least one biomarker selected from CD1C, DLG4, TXNDC15, SOD2, TREML1, IGDCC4, LMNB2, GNAS, CLMP, GFRA2, ARHGEF1, FLRT2, IL15, HAVCR1, FLT3, MAN1A2, ACY3, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, VEGFA, and NEFL.

13. The method of claim 10, wherein a performance of the predictive model is characterized by an AUROC of at least 0.50, at least 0.51, at least 0.64, at least 0.70, at least 0.71, at least 0.72, at least 0.73, at least 0.74, at least 0.75, at least 0.76, at least 0.77, at least 0.79, at least 0.80, at least 0.81, at least 0.82, at least 0.83, at least 0.86, or at least 0.87.

14. The method of claim 1, wherein the prediction of multiple sclerosis disease progression is a prediction of relapse associated worsening (RAW), a prediction of progression independent of relapse activity (PIRA), a measure of expanded disability status scale (EDSS) score, a measure of a patient determined disease steps (PDDS) score, a PRO measurement information system (PROMIS) score, a Multiple Sclerosis Rating Scale, Revised (MSRS-R) score, or a MRI-based volumetric measurement.

15. (canceled)

16. (canceled)

17. The method of claim 14, wherein an expanded disability status scale (EDSS) score less than 6 indicates a mild/moderate MS disease progression and a EDSS score greater than or equal to 6 indicates a severe MS disease progression.

18. (canceled)

19. (canceled)

20. The method of claim 14, wherein a patient determined disease steps (PDDS) score less than or equal to 4 indicates a mild/moderate MS disease progression and a PDDS score greater than 4 indicates a severe MS disease progression.

21-23. (canceled)

24. The method of claim 14, wherein the MRI-based volumetric measurement is one of whole brain atrophy, brain parenchymal fraction (BPF), white matter atrophy, or gray matter atrophy.

25. The method of claim 1, wherein the multiple sclerosis is one of relapsing-remitting multiple sclerosis (RRMS), secondary progressive multiple sclerosis (SPMS), primary-progressive multiple sclerosis (PPMS), progressive relapsing multiple sclerosis (PRMS), or clinically isolated syndrome (CIS).

26-88. (canceled)

89. A non-transitory computer readable medium comprising instructions that, when executed by a processor, cause the processor to:
obtain a dataset comprising expression levels of a plurality of biomarkers, wherein the plurality of biomarkers comprises two or more biomarkers selected from CD1C, DLG4, TXNDC15, SOD2, TREML1, IGDCC4, LMNB2, GNAS, CLMP, GFAP, NEFL, CXCL-13, APLP1, MOG, OPG, VCAN, CDCP1, TNFSF13b, CNTN2, CXCL9, SERPINA9, OPN, CD6, TNFRSF10a, CCL20, FLRT2, COL4A1, GH, IL-12, PRTG, CXCL10, IL15, EGF, CXCL11, CFH, TNFSF10, IL18, IL6, TNF, HAVCR1, FLT3, MAN1A2, ACY3, ARHGEF1, ADGRG1, MYCBP2, ITGB1, CLEC4A, MEP1B, F13B, FCN1, ADCYAP1R1, LILRA5, HEPH, CLEC10A, RABEPK, FCER2, TG, CXCL12, CA3, CXCL8, CCL8, CD22, IL17A, IL7, KLHL41, KLRC1, FCRL1, IL17C, KLKB1, IFNGR2, CST7, FLT3LG, CCL19, GFRA2, SERPINA3, KIRREL1, LTA, AMPD3, CCL2, DPEP2, CFHR5, F10, SERPIND1, CSF3, CCL13, PFKFB2, CSF1, APOF, MMP12, LMOD1, RNASE10, APCS, MMP1, CEP20, NAMPT, OLR1, ADAMTSL2, and VEGFA; and
generate a prediction of multiple sclerosis disease progression by applying a predictive model to the expression levels of the plurality of biomarkers.

90-170. (canceled)
